(19) United States
(12) Patent Application Publication
(10) Pub. No.: US 2026/0232653 A1
(43) Pub. Date: Aug. 13, 2026

(54) USE OF CONDENSED AZACYCLE COMPOUNDS FOR THE TREATMENT OF LYSOSOMAL STORAGE DISORDERS

(71) Applicant: Minerva Neurosciences, Inc., Burlington, MA (US)

(72) Inventors: Remy LUTHRINGER, Geneva (CH); Nadine NOEL, Gildwiller (FR); Florent SCHMITT, Uttenheim (FR); Sandra WERNER, Ostwald (FR); Hans MAAG, Oberammergau (DE); Jay SAOUD, Groton, MA (US)

(21) Appl. No.: 19/157,045

(22) PCT Filed: Feb. 15, 2024
(86) PCT No.: PCT/US2024/016048

§ 371 Date: Aug. 15, 2025

Related U.S. Application Data
(60) Provisional application No. 63/485,449, filed on Feb. 16, 2023.

(51) Int. Cl. A61K 31/4725; A61K 31/472; A61K 31/55; A61K 45/06; A61K 48/00

(52) U.S. Cl. CPC A61K 31/4725; A61K 31/472; A61K 31/55; A61K 45/06; A61K 48/00

(57) ABSTRACT

Described herein are methods for treating or preventing a lysosomal storage disorder (LSD), or a symptom of a lysosomal storage disorder, through the administration of compounds of Formula (I), and their prodrugs, solvates, or pharmaceutically acceptable salts, to a subject in need thereof. Also provided herein are methods for modulating lysosomal size and/or cholesterol accumulation in a subject, through the administration of compounds of Formula (I), their prodrugs, solvates, or pharmaceutically acceptable salts.

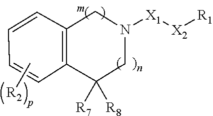

### CROSS REFERENCE TO RELATED APPLICATIONS

This application claims priority to, and the benefit of, U.S. Provisional Application No. 63/485,449, filed Feb. 16, 2023, which is incorporated by reference in its entirety for all purposes.

### FIELD OF THE DISCLOSURE

The present disclosure relates generally to the treatment of lysosomal storage disorders (LSDs), including Niemann-Pick disease.

### BACKGROUND

Lysosomal storage disorders (LSDs) are inherited metabolic disorders where a genetic variation disrupts the normal activity of lysosomes in human cells causing abnormal build-up of various toxic materials in the body's cells due to enzyme deficiencies. It is estimated that LSDs effect about 1 in every 5,000 live births. Although these disorders are considered rare, they are known to effect individuals globally. At present, there are no cures for lysosomal storage disorders and there are not yet specified treatment options for many of the diseases (https://rarediseases.org/rare-diseases/lysosomal-storage-disorders/).

Sigma receptors are implicated in a variety of diseases and disorders, including, for example, pain disorders, neurological disorders (e.g., Parkinson's disease and Alzheimer's disease), and cancer. Improved Sigma receptor modulating compounds may be able to address an unmet need in the treatment of LSDs.

### SUMMARY

In one aspect, the present disclosure provides, inter alia, a method of treating or preventing a lysosomal storage disorder, or a symptom of a lysosomal storage disorder, in a subject in need thereof, wherein the method comprises administering to the subject a therapeutically effective amount of a compound of Formula (I):

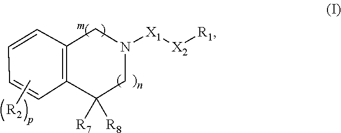

or a prodrug, solvate, or pharmaceutically acceptable salt thereof, wherein
X₁ is absent, —(CR₃R₄)_{q}—, —O—, —S(O)₂—, —C(O)—, —C(S)—, —S(O)—, —NR₃C(O)—, —C(O) NR₃—, —C(O)O—, or —OC(O)—;
X₂ is absent, —(CR₃R₄)_{q}—, —O—, —S(O)₂—, —C(O)—, —C(S)—, —S(O)—, —NR₃C(O)—, —C(O) NR₃—, —C(O)O—, or —OC(O)—;
R₁ is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, 5- to 12-membered heteroaryl comprising 1-6 heteroatoms selected from N, O, and S, 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₅;
each R₂ independently is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆;
each R₃ independently is H, halogen, or C₁-C₆ alkyl;
each R₄ independently is H, halogen, or C₁-C₆ alkyl;
each R₅ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
each R₆ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14 -membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with one or more —C(O) C₁-C₆ alkyl, —C(O)OC₁-C₆ alkyl, —C(O)NH₂, —C(O)NHC₁-C₆ alkyl, or —C(O)N(C₁-C₆alkyl)₂;
R₇ is C₁-C₆ alkyl optionally substituted with one or more OH, halogen, or C₁-C₆ alkoxyl;
R₈ is C₁-C₆ alkyl optionally substituted with one or more OH, halogen, or C₁-C₆ alkoxyl; or
R₇ and R₈, together with the carbon atom they are attached, form a C₃-C₆ cycloalkyl optionally substituted with one or more OH, halogen, or C₁-C₆ alkoxyl;
m is 0, 1, 2, or 3;
n is 0, 1, 2, or 3;
p is 0, 1, 2, 3, or 4; and
each q independently is 1, 2, 3, or 4.

In one aspect, the present disclosure provides, inter alia, a method of treating or preventing a lysosomal storage disorder, or a symptom of a lysosomal storage disorder, in a subject in need thereof, wherein the method comprises administering to the subject a therapeutically effective amount of a compound of Formula (Ia):

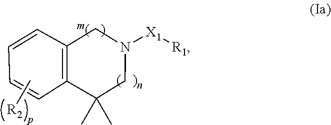

or a prodrug, solvate, or pharmaceutically acceptable salt thereof, wherein
X₁ is a absent, (CR₃R₄)_{q}, —C(O)—, —C(S)—, —S(O)—, —NR₃C(O)—, or —C(O)O—;
R₁ is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl is optionally substituted with one or more R₅;
each R₂ is independently H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆;
each R₃ is independently H or halogen;
each R₄ is independently H or halogen;
each R₅ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
each R₆ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
m is 0, 1, 2, or 3;
n is 0, 1, 2, or 3;
p is 0, 1, 2, 3, or 4; and
q is 1, 2, 3, or 4.

In some embodiments of the present disclosure, the LSD is cholesteryl ester storage disease, Wolman disease, Hunter syndrome, Hurler's disease, Scheie syndrome, Hurler-Scheie syndrome, mucopolysaccharidosis (MPS) type III, mucopolysaccharidosis (MPS) type IV (Morquio syndrome), Maroteaux-Lamy syndrome, Sly syndrome, Fabry disease, Gaucher disease, Krabbe disease, metachromatic leukodystrophy, Niemann-Pick disease, Sandhoff disease, Tay-Sachs disease, alpha mannosidosis, beta mannosidosis, Schindler disease, aspartylglucosaminuria, fucosidosis, neuronal ceroid lipofuscinosis, galactosialidosis, infantile sialic acid storage disease, Salla disease, sialuria, mucolipidosis, cystinosis, Danon disease, multiple sulfatase deficiency, proteins GM2-AP deficiency, Farber disease, lysosomal acid lipase deficiency, hyaluronidase deficiency, pycnodysostosis, and/or Pompe disease.

In some embodiments of the present disclosure, the LSD is Niemann-Pick disease. In some embodiments of the present disclosure, the LSD is Niemann-Pick disease type C. In some embodiments of the present disclosure, the LSD is Niemann-Pick disease type A or B.

In some embodiments of the present disclosure, the subject has dyslipidemia. In some embodiments, the dyslipidemia is high cholesterol. In some embodiments, the dyslipidemia is low cholesterol.

In some embodiments of the present disclosure, the administration of the compound results in a modulation of cholesterol accumulation in the subject; and/or a modulation of the lysosomal size of the subject. In some embodiments, the modulation is an increase in both cholesterol accumulation and lysosomal size. In some embodiments, the modulation is a decrease in both cholesterol accumulation and lysosomal size. In some embodiments, the modulation is an increase in cholesterol accumulation and a decrease in lysosomal size. In some embodiments, the modulation is a decrease of cholesterol accumulation and an increase in lysosomal size.

In some embodiments of the present disclosure, the cholesterol accumulation and/or lysosomal size is modulated by at least 10%, at least 20%, at least 30%, at least 40%, at least 50%, at least 60%, or at least 70%. In some embodiments of the present disclosure, the modulation of cholesterol accumulation and/or lysosomal size occurs within 24 hours, within 48 hours, or within 72 hours of the administration of the compound.

In some embodiments of the present disclosure, the administration of the compound to the subject does not result in a change in NPC1 expression in the subject. In some embodiments of the preceding methods, the subject is a pediatric subject. In some embodiments of the preceding methods, the subject is an adult.

In some embodiments of the present disclosure, the subject is also administered one or more additional LSD treatment agents. In some embodiments, the one or more additional LSD treatment agents is selected from recombinant enzymes, enzymes plus chaperones, recombinant enzymes plus chaperones, fusion proteins (HIRMAb), miglustat, and insulin growth factor II fusion protein.

In some embodiments of the present disclosure, the treatment or prevention of an LSD or a symptom of an LSD further comprises gene therapy. In some embodiments, the therapeutically effective amount of the compound is administered to the subject once per day or twice per day. In some embodiments, the therapeutically effective amount of the compound is between about 1 mg and about 1000 mg.

### BRIEF DESCRIPTION OF THE DRAWINGS

FIGS. 1A-1D show the effect of Compound No. 10 (CB) on NPC1 protein levels. FIG. 1A shows the densitometric analyses (left) and a representative Western blot (right) of NPC1 protein from healthy and I1061T cells. FIGS. 1B-1D show the densitometric analyses (left) and a representative Western blot (right) of NPC1 protein in I1061T fibroblasts treated with Compound No. 10 (CB) plus vehicle (Ctr) for 24 h (FIG. 1B), 48 h (FIG. 1C) and 72 h (FIG. 1D) at the indicated concentrations. 2.5 μM rimcazole (Rim), was used as a positive control of a sigma2/TMEM97 antagonist. The control of protein loading was done with vinculin. The experiments were carried out four times in duplicate. The statistical analysis was performed with Student's t test for FIG. 1A, and one-way ANOVA followed by the Dunnett's multiple comparisons test for FIGS. 1B-1D where *=p<0.05 versus Ctr and **=p<0.001 versus Ctr.

FIG. 2 shows the geo mean ratio of Lysotracker fluorescence in Healthy and I1061T fibroblasts. The experiments were carried out four times in duplicate. The statistical analysis was performed with Student's t test where ^{∘∘∘∘}=p<0.0001 vs Healthy.

FIG. 3 shows the effect of Compound No. 10 (CB) on lysosomes. FIG. 3 shows the geometric (geo) mean ratio of Lysotracker™ fluorescence in healthy and I1061T fibroblasts treated with Compound No. 10 plus vehicle (Ctr) for 24 h (left), 48 h (middle) and 72 h (right) at the indicated concentrations. 2.5 μM Rim was used as a positive control of a sigma2/TMEM97 antagonist at 72 h treatment. 300 μM cyclodextrin (CD) was used as a positive control of lysosomal reduction. The experiments were carried on four times in duplicate. The statistical analysis was performed with one-way ANOVA followed by the Dunnett's multiple comparisons test where *=p<0.05 versus Ctr; **=p<0.001 vs Ctr; and ****=p<0.001 vs Ctr.

### DETAILED DESCRIPTION

Provided herein are, inter alia, methods for treating or preventing a lysosomal storage disorder, or a symptom of a lysosomal storage disorder, in a subject comprising administering a therapeutically or prophylactically effective amount of a compound according to Formula (I). Also provided herein are methods for modulating the lysosomal size and cholesterol accumulation in a subject by administering a therapeutically effective amount of a compound according to Formula (I). Embodiments herein generally relate to methods of treating lysosomal storage disorders (LSDs) and their symptoms.

The methods described herein provide certain advantages over other methods, including for example, the ability to modulate both lysosomal size and cholesterol accumulation. The methods described herein provide novel treatment options for subjects with LSDs, or other diseases with similar symptoms.

### Definitions

The terms used herein have their ordinary meaning and the meaning of such terms is independent at each occurrence thereof. Notwithstanding the foregoing, and except where stated otherwise, the following definitions apply throughout the specification and claims.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₂, C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, “alkoxyl”, “C₁, C₂, C₃, C₄, C₅ or C₆ alkoxyl” or “C₁-C₆ alkoxyl” is intended to include —O—C₁, —O—C₂, —O—C₃, —O—C₄, —O—C₅ or —O—C₆ straight chain (linear) saturated aliphatic hydrocarbon groups and —O—C₂, —O—C₃, —O—C₄, —O—C₅ or —O—C₆ branched saturated aliphatic hydrocarbon groups. For example, C₁-C₆ alkoxyl is intends to include —O—C₁, —O—C₂, —O—C₃, —O—C₄, —O—C₅ and —O—C₆ alkyl groups. Examples of alkoxyl include, moieties having from one to six carbon atoms, such as, but not limited to, methoxyl, ethoxyl, n-propoxyl, i-propoxyl, n-butoxyl, s-butoxyl, t-butoxyl, n-pentoxyl, i-pentoxyl, or n-hexoxyl. In some embodiments, a straight chain or branched alkoxyl has six or fewer carbon atoms (e.g., C₁-C₆ for straight chain, C₃-C₆ for branched chain), and in another embodiment, a straight chain or branched alkoxyl has four or fewer carbon atoms.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

The term “alkylene” or “alkylenyl” refers to a divalent alkyl radical. Any of the above-mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

Optionally substituted moieties (such as optionally substituted alkyl, alkenyl, alkynyl, cycloalkyl, heterocycloalkyl, aryl, heteroaryl, etc.) include both the unsubstituted moieties and the moieties having one or more of the designated substituents. For example, substituted aryl moieties include those which are halogen and/or C₁-C₆ alkyl substituted; substituted heteroaryl moieties include 2,6-dimethylpyridinyl; and substituted heterocycloalkyl includes those substituted with one or more alkyl groups, such as 2,2,6,6-tetramethyl-piperidinyl, and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or another substituent, as defined). The nitrogen and sulphur heteroatoms may optionally be oxidized (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or the corresponding anion, —O⁻.

As used herein, the term “halo” or “halogen” refers to fluorine, chlorine, bromine and iodine.

As used herein, “haloalkyl”, is intended to include “alkyl” moieties that are substituted with one or more halogens. Non-limiting examples include-CH₂F, —CHF₂, —CF₃, —CH₂CF₃, —CH₂CHF₂, —CH₂Cl, —CClF₂, —CCl₃, —CClHCF₃, —CCl₂CHF₂, —CClFCF₃, and —CF₂CCl₃.

As used herein, “haloalkoxyl”, is intended to include “alkoxyl” moieties that are substituted with one or more halogens. Non-limiting examples include —OCH₂F, —OCHF₂, —OCF₃, —OCH₂CF₃, —OCH₂CHF₂, —OCHFCF₃, and —OCF₂CF₃.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. In some embodiments, the compounds of the present disclosure may be prepared as described in WO 2022/036080, which is incorporated by reference herewith in its entirety for all purposes.

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

Unless explicitly indicated otherwise, the terms “approximately” and “about” are synonymous. In some embodiments, “approximately” and “about” refer to the recited amount, value, dose or duration ±20%, ±15%, ±10%, ±8%, ±6%, ±5%, ±4%, ±2%, +1%, or ±0.5%. In another embodiment, “approximately” and “about” refer to the listed amount or duration ±10%, ±8%, ±6%, ±5%, ±4%, or ±2%. In some embodiments, “approximately” and “about” refer to the listed amount, value, dose, or duration ±5%. In some embodiments, “approximately” and “about” refer to the listed amount, value, dose, or duration ±2%. In some embodiments, “approximately” and “about” refer to the listed amount, value, dose, or duration ±1%.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

As used herein, the term “unsubstituted” means that the specified group bears no substituents.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present invention also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

As used herein, “pharmaceutically acceptable salts” and “pharmaceutically acceptable excipients” may be used interchangeably.

As used herein, the terms “patient” or “subject” are used interchangeably.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human. In some embodiments, the subject is a human adult, i.e., 18 years of age or older. In some embodiments, the subject is a human child, i.e., 17 years of age or younger. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

The term “lysosomal storage disorder” or “LSD” is used in this disclosure to refer to an inherited metabolic disorder where a genetic variation disrupts the normal activity of lysosomes in human cells causing abnormal build-up of various toxic materials in the body's cells due to enzyme deficiencies. In some embodiments, the LSD is a lipid storage disorder, a mucopolysaccharidosis, a glycoprotein storage disorder, or a mucolipidosis. In some embodiments, the LSD is an inherited metabolic disorder.

As used herein, “filipin” refers to a polyene macrolide compound that was originally isolated from Streptomyces filipinensis and that exhibits intrinsic fluorescence. Filipin is known in the art as a diagnostic tool for diseases of lipid dysregulation (See, e.g., Dist; et al., The Journal of Pathology, 2003, 200:104-111). Filipin is known in the art to stain unesterified cholesterol. The structure and fluorescent properties of filipin are described in, e.g., in Castanho et al., Eur. J. Biochem., 1992, 207:125-134; Xu et al., J. Biol. Chem., 2010, 285:16844-16853, and WO 2018/107059, the relevant disclosures of which are incorporated by reference for the purpose and subject matter referenced herein.

As used herein, a subject with “high cholesterol”, “hyperlipidemia”, or “hypercholesterolemia” refers to the development of fatty deposits in the blood vessels of the subject. In some embodiments, the accumulation of fatty deposits in the blood vessels limits blood flow. In some embodiments, the accumulation of fatty deposits in the blood vessels increases the subject's risk of heart attack and/or stroke. In some embodiments, high cholesterol is determined by a blood test to quantify cholesterol levels. In some embodiments, the subject is a human, and the high cholesterol is characterized by total serum cholesterol that is greater than or equal to 200 mg/dl or 5.2 mmol/L. In some embodiments, the subject is a human, and the high cholesterol is characterized by low-density lipoprotein (LDL) greater than or equal 70 mg/dL or greater than or equal to 100 mg/dL. In some embodiments, the subject is a human, and the high cholesterol is characterized by LDL greater than or equal to 1.8 mmol/L or greater than or equal to 2.6 mmol/L. In some embodiments, the subject is a human, and the high cholesterol is characterized by triglycerides that are greater than 150 mg/dL or 1.7 mmol/L.

As used herein, an “additional LSD treatment agent”, refers to an agent other than the compound of Formula (I), MIN-101-B, or a pharmaceutically acceptable salt, prodrug, or solvate thereof, that treats or prevents a LSD or a symptom of a LSD. Non-limiting examples of LSD treatment agents include recombinant enzymes, enzymes plus chaperones, recombinant enzymes plus chaperones, fusion proteins (HIRMAb), miglustat, insulin growth factor II fusion protein.

As used herein, “cyclodextrin” (CD) is a cyclic oligosaccharide with a hydrophilic outer surface and a somewhat lipophilic central cavity. In aqueous solutions cyclodextrin can form inclusion complexes with many drugs by taking up a drug molecule, or more frequently some lipophilic moiety of the molecule, into the central cavity. See, e.g., WO 2007/012974, the relevant disclosures of which are incorporated by reference for the purpose and subject matter referenced herein. It has been shown that beta-cyclodextrin reduced cholesterol accumulation and neuronal cell loss in a mouse model of Niemann-Pick Disease, Type C. See, e.g., WO 2014/022841, the relevant disclosures of which are incorporated by reference for the purpose and subject matter referenced herein.

As used herein, “rimcazole” (“Rim”), i.e., 9-[3-(3,5-cis-dimethylpiperazino) propyl]-carbazole, refers to a compound having the following structure:

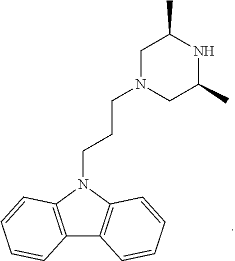 [SMILES: C[C@@H]1CN(CCCn2c3ccccc3c3ccccc32)C[C@H](C)N1]

Rimcazole is known in the art to be an antagonist of the sigma receptor with an IC₅₀ value of 1480 nM and 386 nM for sigma 1 and sigma 2/transmembrane protein 97 (“TMEM97”) receptors, respectively. See, e.g., WO 2001/074359 and Job et al. “A behavioral economic analysis of the effects of rimcazole on reinforcing effects of cocaine injection and food presentation in rats” Psychopharmacology (Berl) 2019 December; 236 (12): 3601-3612, the relevant disclosures of which are incorporated by reference herein. A method for preparing Rimcazole is described in WO2008/040950, the contents of which are incorporated herein in their entirety.

As used herein, “Sigma-2,” “σ-2”, or “Sigma 2” refers to the Sigma-2 receptor. Sigma receptors are a class of receptors that are expressed in many tissues including the central nervous system. It is known in the art that there are two types of sigma receptors: Sigma-1 receptors and Sigma-2 receptors. Compounds which exhibit selective binding to Sigma-2 are known to be associated with dystonia and may block calcium channels. See, e.g., WO 1991/009594A1, the relevant disclosures of which are incorporated by reference for the purpose and subject matter referenced herein.

As used herein, the term “preventing,” “prevent,” or “protecting against” describes reducing or eliminating the onset of the symptoms or complications of such disease, condition or disorder.

It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

In some embodiments, the symptom of the lysosomal storage disorder is hepatosplenomegaly (i.e., an enlarged liver and spleen), ataxia, vertical supranuclear gaze palsy, hypotonia, liver disease, respiratory infection, difficulty with speech, difficulty with swallowing and feeding, loss of cognitive skills, or seizure.

The term “administer”, “administers”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

In some embodiments, the term “therapeutically effective amount,” as used herein, refers to the amount of a compound disclosed herein, or a composition comprising said compound, administered to a patient already suffering from a disease, condition, or disorder, that is sufficient to cure or at least partially arrest, or relieve to some extent one or more of the symptoms of the disease, disorder, or condition being treated.

The effectiveness of such compositions depends upon conditions including, but not limited to, the severity and course of the disease, disorder, or condition, any previous therapy, the patient's health status and response to the drugs, and the judgment of the treating physician. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. Such an amount would be sufficient to elicit the biological or medical response of a tissue system, or patient that is sought by a researcher or clinician. Such a therapeutically effective amount can be determined by one of ordinary skill in the art having regard to their own knowledge, the state of the art, and this disclosure. By way of example only, therapeutically effective amounts may be determined by routine experimentation, including, but not limited to, a dose escalation clinical trial. For any composition described herein, the therapeutically effective amount may be initially determined from cell culture assays.

The term “prophylactically effective amount” of a drug (e.g., the cells described herein) is an amount of the drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations.

“Comprising” or “comprises” as applied to a particular dosage form, composition, method or process described or claimed herein means that the dosage form, composition or method includes all of the recited elements in a specific description or claim but does not exclude other elements. “Consists essentially of” and “consisting essentially of” means that the described or claimed composition, dosage form, method or process does not exclude other materials or steps that do not materially affect the recited physical, pharmacological, pharmacokinetic properties or therapeutic effects of the composition, dosage form, method, or process. “Consists of” and “consisting of” means the exclusion of more than trace elements of other ingredients and substantial method or process steps.

The term “prodrug,” as used in this disclosure, means a compound which is convertible in vivo by metabolic means (e.g., by hydrolysis) to a disclosed compound.

### Compounds of the Present Disclosure

In some aspects, the present disclosure provides, inter alia, a compound of Formula (I):

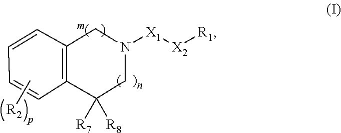

or a prodrug, solvate, or pharmaceutically acceptable salt thereof, wherein
X₁ is absent, —(CR₃R₄)_{q}—, —O—, —S(O)₂—, —C(O)—, —C(S)—, —S(O)—, —NR₃C(O)—, —C(O) NR₃—, —C(O)O—, or —OC(O)—;
X₂ is absent, —(CR₃R₄)_{q}—, —O—, —S(O)₂—, —C(O)—, —C(S)—, —S(O)—, —NR₃— —NR₃C(O)—, —C(O) NR₃—, —C(O)O—, or —OC(O)—;
R₁ is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, 5- to 12-membered heteroaryl comprising 1-6 heteroatoms selected from N, O, and S, 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₅;
each R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆;
each R₃ independently is H, halogen, or C₁-C₆ alkyl;
each R₄ independently is H, halogen, or C₁-C₆ alkyl;
each R₅ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
each R₆ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, aryl, or heterocycloalkyl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
R₇ is C₁-C₆ alkyl optionally substituted with one or more OH, halogen, or C₁-C₆ alkoxyl;
R₈ is C₁-C₆ alkyl optionally substituted with one or more OH, halogen, or C₁-C₆ alkoxyl; or
R₇ and R₈, together with the carbon atom they are attached, form a C₃-C₆ cycloalkyl optionally substituted with one or more OH, halogen, or C₁-C₆ alkoxyl
m is 0, 1, 2, or 3;
n is 0, 1, 2, or 3;
p is 0, 1, 2, 3, or 4; and
each q independently is 1, 2, 3, or 4.

In some aspects, the present disclosure provides, inter alia, a compound of Formula (I):

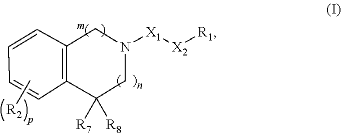

or a prodrug, solvate, or pharmaceutically acceptable salt thereof, wherein
X₁ is absent, —(CR₃R₄)_{q}—, —O—, —S(O)₂—, —C(O)—, —C(S)—, —S(O)—, —NR₃C(O)—, —C(O) NR₃—, —C(O)O—, or —OC(O)—;
X₂ is absent, —(CR₃R₄)_{q}—, —O—, —S(O)₂—, —C(O)—, —C(S)—, —S(O)—, —NR₃— —NR₃C(O)—, —C(O) NR₃—, —C(O)O—, or —OC(O)—;
R₁ is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, 5- to 12-membered heteroaryl comprising 1-6 heteroatoms selected from N, O, and S, 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₅;
each R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆;
each R₃ independently is H, halogen, or C₁-C₆ alkyl;
each R₄ independently is H, halogen, or C₁-C₆ alkyl;
each R₅ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
each R₆ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, aryl, or heterocycloalkyl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
R₇ is C₁-C₆ alkyl;
R₈ is C₁-C₆ alkyl;
m is 0, 1, 2, or 3;
n is 0, 1, 2, or 3;
p is 0, 1, 2, 3, or 4; and
each q independently is 1, 2, 3, or 4.

In some aspects, the present disclosure provides, inter alia, a compound of Formula (Ia):

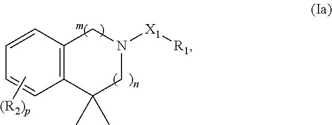

or a prodrug, solvate, or pharmaceutically acceptable salt thereof, wherein
X₁ is a absent, (CR₃R₄)_{q}, —C(O)—, —C(S)—, —S(O)—, —NR₃C(O)—, or —C(O)O—;
R₁ is H, OH, halogen, —NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl is optionally substituted with one or more R₅;
R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S wherein the alkyl, alkenyl, alkynyl, haloalkyl, alkoxyl, haloalkoxyl, cycloalkyl, aryl, heteroaryl, or heterocycloalkyl is optionally substituted with one or more R₆;
each R₃ is H or halogen;
each R₄ is H or halogen;
each R₅ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
each R₆ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S wherein the alkyl, alkenyl, alkynyl, cycloalkyl, aryl, or heterocycloalkyl is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
m is 0, 1, 2, or 3;
n is 0, 1, 2, or 3;
p is 0, 1, 2, 3, or 4; and
each q independently is 1, 2, 3, or 4.

In some embodiments, X¹ is absent, (CR₃R₄)_{q}, —O—, —S(O)₂—, —C(O)—, —C(S)—, —S(O)—, —NR₃C(O)—, —C(O) NR₃—, —C(O)O—, or —OC(O)—.

In some embodiments, X¹ is-O—, —S(O)₂—, —NR₃C(O)—, or —C(O) NR₃—.

In some embodiments, X¹ is a absent, —(CR₃R₄)_{q}—, —C(O)—, —C(S)—, —S(O)—, —NR₃C(O)—, or —C(O)O—.

In some embodiments, X¹ is a absent, —(CR₃R₄)_{q}—, —C(O)—, or —C(O)O—.

In some embodiments, X¹ is a absent.

In some embodiments, X¹ is —(CR₃R₄)_{q}—.

In some embodiments, X¹ is —C(O)— or —C(O)O—.

In some embodiments, X¹ is —C(O)—.

In some embodiments, X¹ is —C(O)O—.

In some embodiments, X¹ is —NR₃C(O)— or —C(O) NR₃—.

In some embodiments, X¹ is —O— or —S(O)₂—. In some embodiments, X¹ is-O—. In some embodiments, X¹ is —S(O)₂—. In some embodiments, X₂ is absent, —(CR₃R₄)_{q}—, —O—, —S(O)₂—, —C(O)—, —C(S)—, —S(O)—, —NR₃C(O)—, —C(O) NR₃—, —C(O)O—, or —OC(O)—.

In some embodiments, X₂ is-O—, —S(O)₂—, —NR₃C(O)—, or —C(O) NR₃—.

In some embodiments, X₂ is absent, —(CR₃R₄)_{q}—, —C(O)—, —C(S)—, —S(O)—, —NR₃C(O)—, or —C(O)O—.

In some embodiments, X₂ is absent, —(CR₃R₄)_{q}—, —C(O)—, or —C(O)O—.

In some embodiments, X₂ is absent.

In some embodiments, X₂ is —(CR₃R₄)_{q}—.

In some embodiments, X₂ is —C(O)— or —C(O)O—.

In some embodiments, X₂ is —C(O)—.

In some embodiments, X₂ is —C(O)O—.

In some embodiments, X₂ is —NR₃C(O)— or —C(O) NR₃—.

In some embodiments, X₂ is —O— or —S(O)₂—. In some embodiments, X₂ is-O—. In some embodiments, X₂ is —S(O)₂—.

In some embodiments, Ri is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, 5- to 12-membered heteroaryl comprising 1-6 heteroatoms selected from N, O, and S, 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₅.

In some embodiments, R₁ is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is optionally substituted with one or more R₅.

In some embodiments, R₁ is H or OH. In some embodiments, R₁ is H. In some embodiments, R₁ is OH.

In some embodiments, R₁ is halogen.

In some embodiments, R₁ is NH₂.

In some embodiments, R₁ is C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is optionally substituted with one or more R₅.

In some embodiments, R₁ is C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is substituted with one or more R₅.

In some embodiments, R₁ is C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is substituted with one R₅.

In some embodiments, R₁ is C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is substituted with two R₅.

In some embodiments, Ri is C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is substituted with three R₅.

In some embodiments, R₁ is C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl.

In some embodiments, R₁ is C₁-C₆ alkyl, C₂-C₆ alkenyl, or C₂-C₆ alkynyl.

In some embodiments, R₁ is C₁-C₆ alkyl. In some embodiments, R₁ is methyl. In some embodiments, R₁ is ethyl. In some embodiments, R₁ is propyl. In some embodiments, R₁ is butyl. In some embodiments, R₁ is isopropyl. In some embodiments, R₁ is iso-butyl. In some embodiments, R₁ is sec-butyl. In some embodiments, R₁ is tert-butyl. In some embodiments, R₁ is pentyl. In some embodiments, R₁ is iso-pentyl. In some embodiments, R₁ is hexyl. In some embodiments, R₁ is iso-hexyl.

In some embodiments, R₁ is C₂-C₆ alkenyl. In some embodiments, R₁ is C₂ alkenyl. In some embodiments, R₁ is C₃ alkenyl. In some embodiments, R₁ is C₄ alkenyl. In some embodiments, R₁ is Cs alkenyl. In some embodiments, R₁ is C₆ alkenyl.

In some embodiments, R₁ is C₂-C₆ alkynyl. In some embodiments, R₁ is C₂ alkynyl. In some embodiments, R₁ is C₃ alkynyl. In some embodiments, R₁ is C₄ alkynyl. In some embodiments, R₁ is Cs alkynyl. In some embodiments, R₁ is C₆ alkynyl.

In some embodiments, R₁ is C₁-C₆ haloalkyl, C₁-C₆ alkoxyl, C₁-C₆ haloalkoxyl, or C₃-C₆ cycloalkyl.

In some embodiments, R₁ is C₁-C₆ haloalkyl. In some embodiments, R₁ is halomethyl. In some embodiments, R₁ is haloethyl. In some embodiments, R₁ is halopropyl. In some embodiments, R₁ is halobutyl. In some embodiments, R₁ is halopentyl. In some embodiments, R₁ is halohexyl.

In some embodiments, R₁ is C₁-C₆ alkoxyl. In some embodiments, R₁ is methoxyl. In some embodiments, R₁ is ethoxyl. In some embodiments, R₁ is propoxyl. In some embodiments, R₁ is butoxyl. In some embodiments, R₁ is pentoxyl. In some embodiments, R₁ is hexoxyl.

In some embodiments, R₁ is C₁-C₆ haloalkoxyl. In some embodiments, R₁ is halomethoxyl. In some embodiments, R₁ is haloethoxyl. In some embodiments, R₁ is halopropoxyl. In some embodiments, R₁ is halobutoxyl. In some embodiments, R₁ is halopentoxyl. In some embodiments, R₁ is halohexoxyl.

In some embodiments, R₁ is C₃-C₆ cycloalkyl. In some embodiments, R₁ is cyclopropyl. In some embodiments, R₁ is cyclobutyl. In some embodiments, R₁ is cyclopentyl. In some embodiments, R₁ is cyclohexyl.

In some embodiments, R₁ is C₁-C₆ alkyl, C₂-C₆ alkenyl, or C₂-C₆ alkynyl, wherein the alkyl, alkenyl, or alkynyl of any of the foregoing is optionally substituted with one or more R₅.

In some embodiments, R₁ is C₁-C₆ alkyl, C₂-C₆ alkenyl, or C₂-C₆ alkynyl, wherein the alkyl, alkenyl, or alkynyl of any of the foregoing is substituted with one or more R₅.

In some embodiments, R₁ is C₁-C₆ alkyl, C₂-C₆ alkenyl, or C₂-C₆ alkynyl, wherein the alkyl, alkenyl, or alkynyl of any of the foregoing is substituted with one R₅.

In some embodiments, R₁ is C₁-C₆ alkyl, C₂-C₆ alkenyl, or C₂-C₆ alkynyl, wherein the alkyl, alkenyl, or alkynyl of any of the foregoing is substituted with two R₅.

In some embodiments, R₁ is C₁-C₆ alkyl, C₂-C₆ alkenyl, or C₂-C₆ alkynyl, wherein the alkyl, alkenyl, or alkynyl is substituted with three R₅.

In some embodiments, R₁ is methyl substituted with one or more R₅. In some embodiments, R₁ is ethyl substituted with one or more R₅. In some embodiments, R₁ is propyl substituted with one or more R₅. In some embodiments, R₁ is butyl substituted with one or more R₅. In some embodiments, R₁ is pentyl substituted with one or more R₅. In some embodiments, R₁ is hexyl substituted with one or more R₅.

In some embodiments, R₁ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S, wherein the aryl, heteroaryl, or heterocycloalkyl of any of the foregoing is optionally substituted with one or more R₅.

In some embodiments, R₁ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S.

In some embodiments, R₁ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S, wherein the aryl, heteroaryl, or heterocycloalkyl of any of the foregoing is substituted with one or more R₅.

In some embodiments, R₁ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S, wherein the aryl, heteroaryl, or heterocycloalkyl of any of the foregoing is substituted with one R₅.

In some embodiments, R₁ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S, wherein the aryl, heteroaryl, or heterocycloalkyl of any of the foregoing is substituted with two R₅.

In some embodiments, R₁ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S, wherein the aryl, heteroaryl, or heterocycloalkyl of any of the foregoing is substituted with three R₅.

In some embodiments, R₁ is H, OH, methyl,

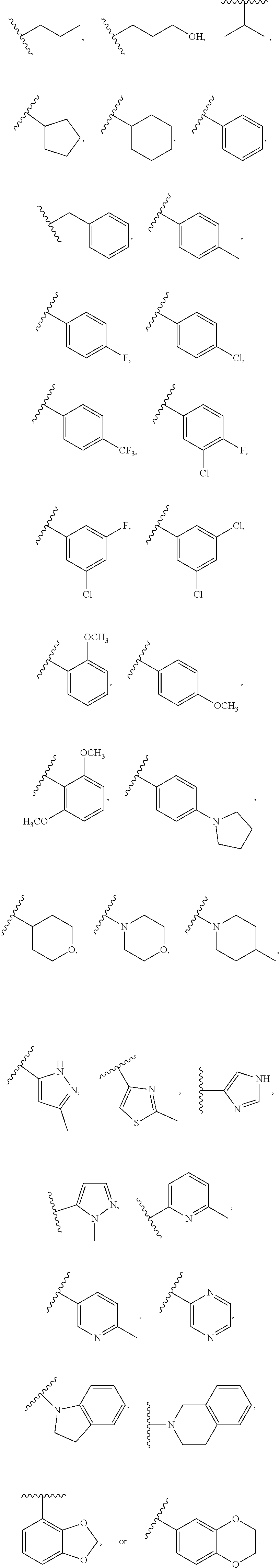

In some embodiments, R₁ is H, OH, methyl,

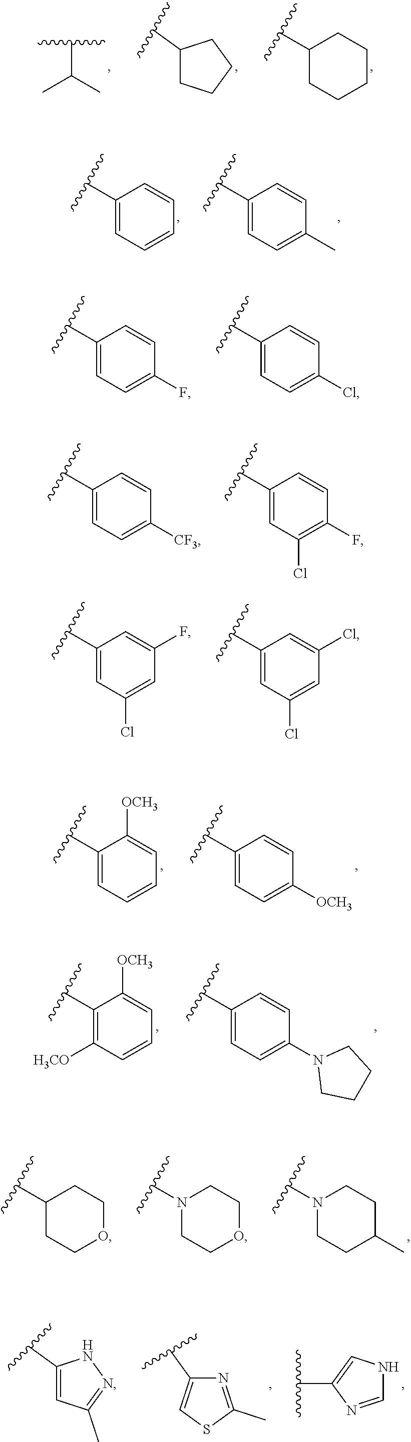

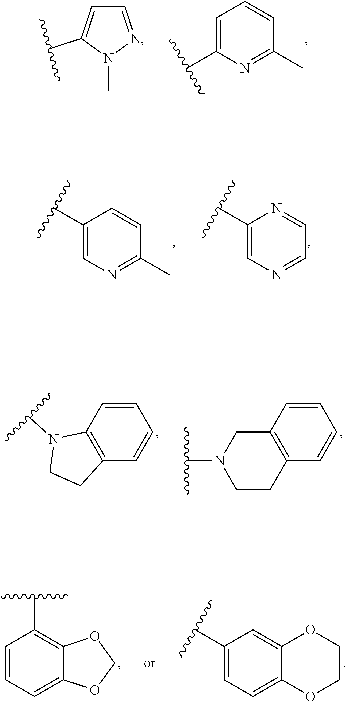

In some embodiments, R₁ is

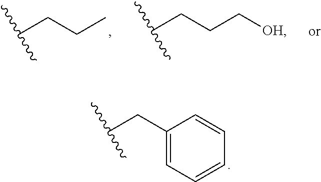

In some embodiments, R₁ is H, OH, methyl,

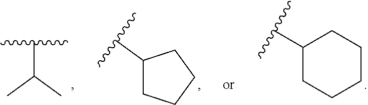

In some embodiments, R₁ is

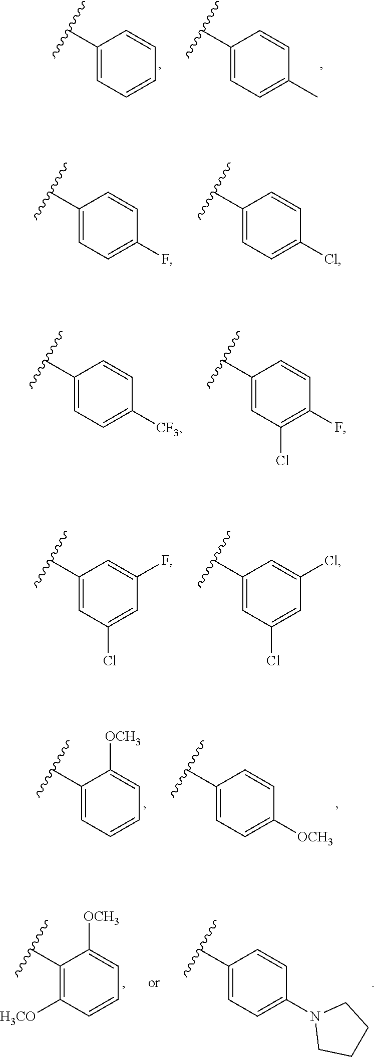

In some embodiments, R₁ is

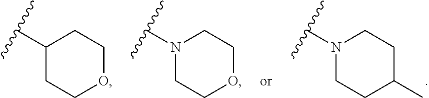

In some embodiments, R₁ is

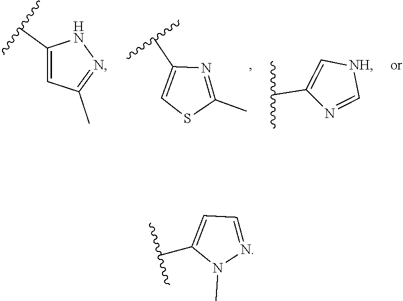

In some embodiments, R₁ is

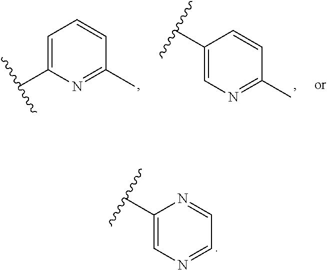

In some embodiments, R₁ is

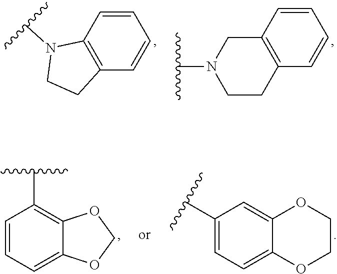

In some embodiments, R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S wherein the alkyl, alkenyl, alkynyl, haloalkyl, alkoxyl, haloalkoxyl, cycloalkyl, aryl, heteroaryl, or heterocycloalkyl is optionally substituted with one or more R₆.

In some embodiments, R₂ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆.

In some embodiments, R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is substituted with one or more R₆.

In some embodiments, R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is substituted with one R₆.

In some embodiments, R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is substituted with two R₆.

In some embodiments, R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is substituted with three R₆.

In some embodiments, R₂ is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S.

In some embodiments, R₂ is H, OH, or halogen. In some embodiments, R₂ is H. In some embodiments, R₂ is OH. In some embodiments, R₂ is halogen. In some embodiments, R₂ is fluorine. In some embodiments, R₂ is chlorine. In some embodiments, R₂ is bromine. In some embodiments, R₂ is iodine.

In some embodiments, R₂ is C₁-C₆ alkyl, C₂-C₆ alkenyl, or C₂-C₆ alkynyl.

In some embodiments, R₂ is C₁-C₆ alkyl. In some embodiments, R₂ is methyl. In some embodiments, R₂ is ethyl. In some embodiments, R₂ is propyl. In some embodiments, R₂ is butyl. In some embodiments, R₂ is isopropyl. In some embodiments, R₂ is iso-butyl. In some embodiments, R₂ is sec-butyl. In some embodiments, R₂ is tert-butyl. In some embodiments, R₂ is pentyl. In some embodiments, R₂ is iso-pentyl. In some embodiments, R₂ is hexyl. In some embodiments, R₂ is iso-hexyl.

In some embodiments, R₂ is C₂-C₆ alkenyl. In some embodiments, R₂ is C₂ alkenyl. In some embodiments, R₂ is C₃ alkenyl. In some embodiments, R₂ is C₄ alkenyl. In some embodiments, R₂ is Cs alkenyl. In some embodiments, R₂ is C₆ alkenyl.

In some embodiments, R₂ is C₂-C₆ alkynyl. In some embodiments, R₂ is C₂ alkynyl. In some embodiments, R₂ is C₃ alkynyl. In some embodiments, R₂ is C₄ alkynyl. In some embodiments, R₂ is Cs alkynyl. In some embodiments, R₂ is C₆ alkynyl.

In some embodiments, R₂ is C₁-C₆ haloalkyl, C₁-C₆ alkoxyl, C₁-C₆ haloalkoxyl, or C₃-C₆ cycloalkyl.

In some embodiments, R₂ is C₁-C₆ haloalkyl. In some embodiments, R₂ is halomethyl. In some embodiments, R₂ is haloethyl. In some embodiments, R₂ is halopropyl. In some embodiments, R₂ is halobutyl. In some embodiments, R₂ is halopentyl. In some embodiments, R₂ is halohexyl.

In some embodiments, R₂ is C₁-C₆ alkoxyl. In some embodiments, R₂ is methoxyl. In some embodiments, R₂ is ethoxyl. In some embodiments, R₂ is propoxyl. In some embodiments, R₂ is butoxyl. In some embodiments, R₂ is pentoxyl. In some embodiments, R₂ is hexoxyl.

In some embodiments, R₂ is C₁-C₆ haloalkoxyl. In some embodiments, R₂ is halomethoxyl. In some embodiments, R₂ is haloethoxyl. In some embodiments, R₂ is halopropoxyl. In some embodiments, R₂ is halobutoxyl. In some embodiments, R₂ is halopentoxyl. In some embodiments, R₂ is halohexoxyl.

In some embodiments, R₂ is C₃-C₆ cycloalkyl. In some embodiments, R₂ is cyclopropyl. In some embodiments, R₂ is cyclobutyl. In some embodiments, R₂ is cyclopentyl. In some embodiments, R₂ is cyclohexyl.

In some embodiments, R₂ is —C(O)OC₁-C₆alkyl, —C(O)NH₂, —C(O)NHC₁-C₆alkyl, or —C(O)N(C₁-C₆alkyl)₂. In some embodiments, R₂ is —C(O)OC₁-C₆alkyl. In some embodiments, R₂ is —C(O)NH₂. In some embodiments, R₂ is —C(O)NHC₁-C₆alkyl. In some embodiments, R₂ is —C(O)N(C₁-C₆alkyl)₂.

In some embodiments, R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl is optionally substituted with one or more R₆.

In some embodiments, R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is substituted with one or more R₆.

In some embodiments, R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is substituted with one R₆.

In some embodiments, R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is substituted with two R₆.

In some embodiments, R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is substituted with three R₆.

In some embodiments, R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S.

In some embodiments, R₂ is C₆-C₁₀ aryl. In some embodiments, R₂ is C₆ aryl. In some embodiments, R₂ is C₇ aryl. In some embodiments, R₂ is Cs aryl. In some embodiments, R₂ is C₉ aryl. In some embodiments, R₂ is C₁₀ aryl.

In some embodiments, R₂ is C₆-C₁₀ aryl, wherein the aryl is substituted with one or more R₆.

In some embodiments, R₂ is C₆-C₁₀ aryl, wherein the aryl is substituted with one R₆.

In some embodiments, R₂ is C₆-C₁₀ aryl, wherein the aryl is substituted with two R₆.

In some embodiments, R₂ is C₆-C₁₀ aryl, wherein the aryl is substituted with three R₆.

In some embodiments, R₂ is 5- to 12-membered heteroaryl comprising 1-6 heteroatoms selected from N, O, and S.

In some embodiments, R₂ is 5- to 12-membered heteroaryl comprising 1-6 heteroatoms selected from N, O, and S, wherein the heteroaryl is substituted with one or more R₆.

In some embodiments, R₂ is 5- to 12-membered heteroaryl comprising 1-6 heteroatoms selected from N, O, and S, wherein the heteroaryl is substituted with one R₆.

In some embodiments, R₂ is 5- to 12-membered heteroaryl comprising 1-6 heteroatoms selected from N, O, and S, wherein the heteroaryl is substituted with two R₆.

In some embodiments, R₂ is 5- to 12-membered heteroaryl comprising 1-6 heteroatoms selected from N, O, and S, wherein the heteroaryl is substituted with three R₆.

In some embodiments, R₂ is 5- to 14-membered saturated or unsaturated heterocycloalkyl comprising 1-5 heteroatoms selected from N, O, and S.

In some embodiments, R₂ is 5- to 14-membered saturated or unsaturated heterocycloalkyl comprising 1-5 heteroatoms selected from N, O, and S, wherein the heterocycloalkyl is substituted with one or more R₆.

In some embodiments, R₂ is 5- to 14-membered saturated or unsaturated heterocycloalkyl comprising 1-5 heteroatoms selected from N, O, and S, wherein the heterocycloalkyl is substituted with one R₆.

In some embodiments, R₂ is 5- to 14-membered saturated or unsaturated heterocycloalkyl comprising 1-5 heteroatoms selected from N, O, and S, wherein the heterocycloalkyl is substituted with two R₆.

In some embodiments, R₂ is 5- to 14-membered saturated or unsaturated heterocycloalkyl comprising 1-5 heteroatoms selected from N, O, and S, wherein the heterocycloalkyl is substituted with three R₆.

In some embodiments, R₂ is 5- to 14-membered saturated heterocycloalkyl comprising 1-5 heteroatoms selected from N, O, and S.

In some embodiments, R₂ is 6-membered saturated heterocycloalkyl comprising 1-5 heteroatoms selected from N, O, and S.

In some embodiments, R₂ is 6-membered saturated heterocycloalkyl comprising 2 heteroatoms selected from N, O, and S.

In some embodiments, R₂ is 6-membered saturated heterocycloalkyl comprising 2 N atoms.

In some embodiments, R₂ is

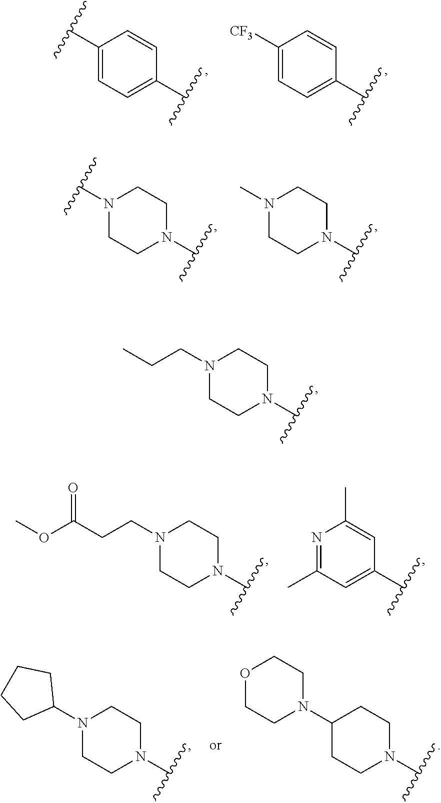

In some embodiments, R₂ is

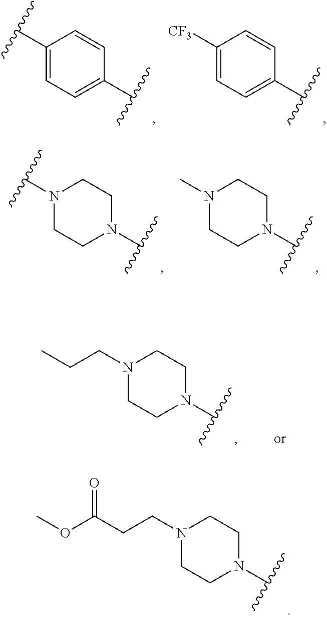

In some embodiments, R₂ is

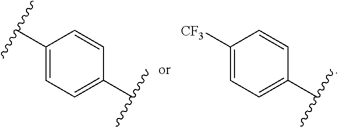 [SMILES: *c1ccc(*)cc1 ; *c1ccc(C(F)(F)F)cc1]

In some embodiments, R₂ is

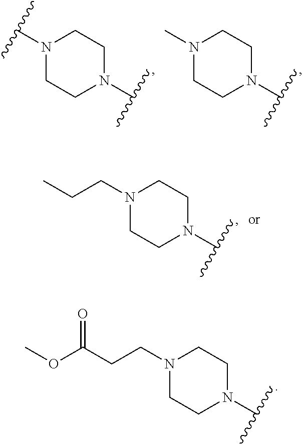

In some embodiments, R₂ is

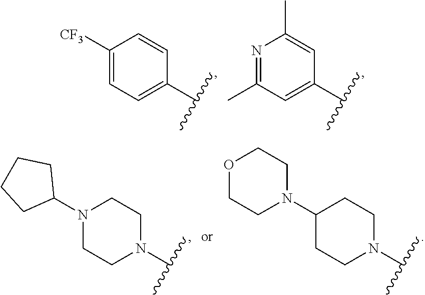

In some embodiments, R₃ is H or halogen. In some embodiments, R₃ is H. In some embodiments, R₃ is halogen.

In some embodiments, R₄ is H or halogen. In some embodiments, R₄ is H. In some embodiments, R₄ is halogen.

In some embodiments, m is 0, 1, 2, or 3. In some embodiments, m is 0. In some embodiments, m is 1. In some embodiments, m is 2. In some embodiments, m is 3.

In some embodiments, n is 0, 1, 2, or 3. In some embodiments, n is 0. In some embodiments, n is 1. In some embodiments, n is 2. In some embodiments, n is 3.

In some embodiments, m is 1 and n is 1.

In some embodiments, m is 1 and n is 2.

In some embodiments, p is 0, 1, 2, 3, or 4. In some embodiments, p is 0. In some embodiments, p is 1. In some embodiments, p is 2. In some embodiments, p is 3. In some embodiments, p is 4.

In some embodiments, q is 1, 2, 3, or 4. In some embodiments, q is 1. In some embodiments, q is 2. In some embodiments, q is 3. In some embodiments, q is 4.

In some embodiments, R₅ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₅ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is substituted with —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₅ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₅ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl of any of the foregoing is substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₅ is OH or halogen. In some embodiments, R₅ is OH. In some embodiments, R₅ is halogen. In some embodiments, R₅ is fluorine. In some embodiments, R₅ is chlorine. In some embodiments, R₅ is bromine. In some embodiments, R₅ is iodine.

In some embodiments, R₅ is C₁-C₆ alkyl, C₂-C₆ alkenyl, or C₂-C₆ alkynyl.

In some embodiments, R₅ is C₁-C₆ alkyl. In some embodiments, R₅ is methyl. In some embodiments, R₅ is ethyl. In some embodiments, R₅ is propyl. In some embodiments, R₅ is butyl. In some embodiments, R₅ is isopropyl. In some embodiments, R₅ is iso-butyl. In some embodiments, R₅ is sec-butyl. In some embodiments, R₅ is tert-butyl. In some embodiments, R₅ is pentyl. In some embodiments, R₅ is iso-pentyl. In some embodiments, R₅ is hexyl. In some embodiments, R₅ is iso-hexyl.

In some embodiments, R₅ is C₂-C₆ alkenyl. In some embodiments, R₅ is C₂ alkenyl. In some embodiments, R₅ is C₃ alkenyl. In some embodiments, R₅ is C₄ alkenyl. In some embodiments, R₅ is Cs alkenyl. In some embodiments, R₅ is C₆ alkenyl.

In some embodiments, R₅ is C₂-C₆ alkynyl. In some embodiments, R₅ is C₂ alkynyl. In some embodiments, R₅ is C₃ alkynyl. In some embodiments, R₅ is C₄ alkynyl. In some embodiments, R₅ is Cs alkynyl. In some embodiments, R₅ is C₆ alkynyl.

In some embodiments, R₅ is C₁-C₆ haloalkyl, C₁-C₆ alkoxyl, C₁-C₆ haloalkoxyl, or C₃-C₆ cycloalkyl.

In some embodiments, R₅ is C₁-C₆ haloalkyl. In some embodiments, R₅ is halomethyl. In some embodiments, R₅ is haloethyl. In some embodiments, R₅ is halopropyl. In some embodiments, R₅ is halobutyl. In some embodiments, R₅ is halopentyl. In some embodiments, R₅ is halohexyl.

In some embodiments, R₅ is C₁-C₆ alkoxyl. In some embodiments, R₅ is methoxyl. In some embodiments, R₅ is ethoxyl. In some embodiments, R₅ is propoxyl. In some embodiments, R₅ is butoxyl. In some embodiments, R₅ is pentoxyl. In some embodiments, R₅ is hexoxyl.

In some embodiments, R₅ is C₁-C₆ haloalkoxyl. In some embodiments, R₅ is halomethoxyl. In some embodiments, R₅ is haloethoxyl. In some embodiments, R₅ is halopropoxyl. In some embodiments, R₅ is halobutoxyl. In some embodiments, R₅ is halopentoxyl. In some embodiments, R₅ is halohexoxyl.

In some embodiments, R₅ is C₃-C₆ cycloalkyl. In some embodiments, R₅ is cyclopropyl. In some embodiments, R₅ is cyclobutyl. In some embodiments, R₅ is cyclopentyl. In some embodiments, R₅ is cyclohexyl.

In some embodiments, R₅ is —C(O)OC₁-C₆alkyl, —C(O)NH₂, —C(O)NHC₁-C₆alkyl, or —C(O)N(C₁-C₆alkyl)₂. In some embodiments, R₅ is —C(O)OC₁-C₆alkyl. In some embodiments, R₅ is —C(O)NH₂. In some embodiments, R₅ is —C(O)NHC₁-C₆alkyl. In some embodiments, R₅ is —C(O)N(C₁-C₆alkyl)₂.

In some embodiments, R₅ is alkyl substituted with —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₅ is alkyl substituted with —C(O)OC₁-C₆alkyl. In some embodiments, R₅ is alkyl substituted with —C(O)Omethyl. In some embodiments, R₅ is alkyl substituted with —C(O)Oethyl. In some embodiments, R₅ is alkyl substituted with —C(O)Opropyl. In some embodiments, R₅ is alkyl substituted with —C(O)Oisopropyl. In some embodiments, R₅ is alkyl substituted with —C(O)Obutyl. In some embodiments, R₅ is alkyl substituted with —C(O)Opentyl. In some embodiments, R₅ is alkyl substituted with —C(O)Ohexyl.

In some embodiments, R₆ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, aryl, or heterocycloalkyl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₆ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, aryl, or heterocycloalkyl of any of the foregoing is substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₆ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₆ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl of any of the foregoing is substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₆ is OH or halogen. In some embodiments, R₆ is OH. In some embodiments, R₆ is halogen. In some embodiments, R₆ is fluorine. In some embodiments, R₆ is chlorine. In some embodiments, R₆ is bromine. In some embodiments, R₆ is iodine.

In some embodiments, R₆ is C₁-C₆ alkyl, C₂-C₆ alkenyl, or C₂-C₆ alkynyl.

In some embodiments, R₆ is C₁-C₆ alkyl. In some embodiments, R₆ is methyl. In some embodiments, R₆ is ethyl. In some embodiments, R₆ is propyl. In some embodiments, R₆ is butyl. In some embodiments, R₆ is isopropyl. In some embodiments, R₆ is iso-butyl. In some embodiments, R₆ is sec-butyl. In some embodiments, R₆ is tert-butyl. In some embodiments, R₆ is pentyl. In some embodiments, R₆ is iso-pentyl. In some embodiments, R₆ is hexyl. In some embodiments, R₆ is iso-hexyl.

In some embodiments, R₆ is C₂-C₆ alkenyl. In some embodiments, R₆ is C₂ alkenyl. In some embodiments, R₆ is C₃ alkenyl. In some embodiments, R₆ is C₄ alkenyl. In some embodiments, R₆ is Cs alkenyl. In some embodiments, R₆ is C₆ alkenyl.

In some embodiments, R₆ is C₂-C₆ alkynyl. In some embodiments, R₆ is C₂ alkynyl. In some embodiments, R₆ is C₃ alkynyl. In some embodiments, R₆ is C₄ alkynyl. In some embodiments, R₆ is Cs alkynyl. In some embodiments, R₆ is C₆ alkynyl.

In some embodiments, R₆ is C₁-C₆ haloalkyl, C₁-C₆ alkoxyl, C₁-C₆ haloalkoxyl, or C₃-C₆ cycloalkyl.

In some embodiments, R₆ is C₁-C₆ haloalkyl. In some embodiments, R₆ is halomethyl. In some embodiments, R₆ is haloethyl. In some embodiments, R₆ is halopropyl. In some embodiments, R₆ is halobutyl. In some embodiments, R₆ is halopentyl. In some embodiments, R₆ is halohexyl.

In some embodiments, R₆ is C₁-C₆ alkoxyl. In some embodiments, R₆ is methoxyl. In some embodiments, R₆ is ethoxyl. In some embodiments, R₆ is propoxyl. In some embodiments, R₆ is butoxyl. In some embodiments, R₆ is pentoxyl. In some embodiments, R₆ is hexoxyl.

In some embodiments, R₆ is C₁-C₆ haloalkoxyl. In some embodiments, R₆ is halomethoxyl. In some embodiments, R₆ is haloethoxyl. In some embodiments, R₆ is halopropoxyl. In some embodiments, R₆ is halobutoxyl. In some embodiments, R₆ is halopentoxyl. In some embodiments, R₆ is halohexoxyl.

In some embodiments, R₆ is C₃-C₆ cycloalkyl. In some embodiments, R₆ is cyclopropyl. In some embodiments, R₆ is cyclobutyl. In some embodiments, R₆ is cyclopentyl. In some embodiments, R₆ is cyclohexyl.

In some embodiments, R₆ is —C(O)OC₁-C₆alkyl, —C(O)NH₂, —C(O)NHC₁-C₆alkyl, or —C(O)N(C₁-C₆alkyl)₂. In some embodiments, R₆ is —C(O)OC₁-C₆alkyl. In some embodiments, R₆ is —C(O)NH₂. In some embodiments, R₆ is —C(O)NHC₁-C₆alkyl. In some embodiments, R₆ is —C(O)N(C₁-C₆alkyl)₂.

In some embodiments, R₆ is alkyl substituted with —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₆ is alkyl substituted with —C(O)OC₁-C₆alkyl. In some embodiments, R₆ is alkyl substituted with —C(O)Omethyl. In some embodiments, R₆ is alkyl substituted with —C(O)Oethyl. In some embodiments, R₆ is alkyl substituted with —C(O)Opropyl. In some embodiments, R₆ is alkyl substituted with —C(O)Oisopropyl. In some embodiments, R₆ is alkyl substituted with —C(O)Obutyl. In some embodiments, R₆ is alkyl substituted with —C(O)Opentyl. In some embodiments, R₆ is alkyl substituted with —C(O)Ohexyl.

In some embodiments, R₆ is a 5- to 14-membered saturated or unsaturated heterocycloalkyl comprising 1-5 heteroatoms selected from N, O, and S wherein the heterocycloalkyl is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₆ is a 5- to 14-membered saturated or unsaturated heterocycloalkyl comprising 1-5 heteroatoms selected from N, O, and S wherein the heterocycloalkyl is substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

In some embodiments, R₆ is a 5- to 14-membered saturated or unsaturated heterocycloalkyl comprising 1 heteroatom selected from N, O, and S.

In some embodiments, R₆ is a 5- to 14-membered saturated or unsaturated heterocycloalkyl comprising 2 heteroatoms selected from N, O, and S.

In some embodiments, R₆ is a 5- to 14-membered saturated or unsaturated heterocycloalkyl comprising 3 heteroatoms selected from N, O, and S.

In some embodiments, R₆ is a 5- to 14-membered saturated or unsaturated heterocycloalkyl comprising 4 heteroatoms selected from N, O, and S.

In some embodiments, R₆ is a 5- to 14-membered saturated or unsaturated heterocycloalkyl comprising 5 heteroatoms selected from N, O, and S.

In some embodiments, R₆ is a 5-membered saturated or unsaturated heterocycloalkyl comprising 2 heteroatoms selected from N, O, and S.

In some embodiments, R₆ is a 6-membered saturated or unsaturated heterocycloalkyl comprising 2 heteroatoms selected from N, O, and S.

In some embodiments, R₆ is a 7-membered saturated or unsaturated heterocycloalkyl comprising 2 heteroatoms selected from N, O, and S.

In some embodiments, R₆ is an 8-membered saturated or unsaturated heterocycloalkyl comprising 2 heteroatoms selected from N, O, and S.

In some embodiments, R₆ is a 9-membered saturated or unsaturated heterocycloalkyl comprising 2 heteroatoms selected from N, O, and S.

In some embodiments, R₆ is a 10-membered saturated or unsaturated heterocycloalkyl comprising 2 heteroatoms selected from N, O, and S.

In some embodiments, R₆ is a 11-membered saturated or unsaturated heterocycloalkyl comprising 2 heteroatoms selected from N, O, and S.

In some embodiments, R₆ is a 12-membered saturated or unsaturated heterocycloalkyl comprising 2 heteroatoms selected from N, O, and S.

In some embodiments, R₆ is a 13-membered saturated or unsaturated heterocycloalkyl comprising 2 heteroatoms selected from N, O, and S.

In some embodiments, R₆ is a 14-membered saturated or unsaturated heterocycloalkyl comprising 2 heteroatoms selected from N, O, and S.

In some embodiments, R₆ is

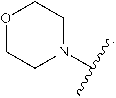 [SMILES: *N1CCOCC1]

In some embodiments, R₇ is C₁-C₆ alkyl optionally substituted with one or more OH, halogen, or C₁-C₆ alkoxyl. In some embodiments, R₇ is methyl optionally substituted with one or more OH, halogen, or C₁-C₆ alkoxyl. In some embodiments, R₇ is ethyl optionally substituted with one or more OH, halogen, or C₁-C₆ alkoxyl. In some embodiments, R₇ is propyl optionally substituted with one or more OH, halogen, or C₁-C₆ alkoxyl. In some embodiments, R₇ is butyl optionally substituted with one or more OH, halogen, or C₁-C₆ alkoxyl. In some embodiments, R_{z} is pentyl optionally substituted with one or more OH, halogen, or C₁-C₆ alkoxyl. In some embodiments, R_{z} is hexyl optionally substituted with one or more OH, halogen, or C₁-C₆ alkoxyl.

In some embodiments, R₈ is C₁-C₆ alkyl optionally substituted with one or more OH, halogen, or C₁-C₆ alkoxyl. In some embodiments, R₈ is methyl optionally substituted with one or more OH, halogen, or C₁-C₆ alkoxyl. In some embodiments, R₈ is ethyl optionally substituted with one or more OH, halogen, or C₁-C₆ alkoxyl. In some embodiments, R₈ is propyl optionally substituted with one or more OH, halogen, or C₁-C₆ alkoxyl. In some embodiments, R₇ is butyl optionally substituted with one or more OH, halogen, or C₁-C₆ alkoxyl. In some embodiments, R₈ is pentyl optionally substituted with one or more OH, halogen, or C₁-C₆ alkoxyl. In some embodiments, R₈ is hexyl optionally substituted with one or more OH, halogen, or C₁-C₆ alkoxyl.

In some embodiments, R₇ is C₁-C₆ alkyl. In some embodiments, R₇ is methyl. In some embodiments, R₇ is ethyl. In some embodiments, R₇ is propyl. In some embodiments, R₇ is butyl. In some embodiments, R₇ is pentyl. In some embodiments, R₇ is hexyl.

In some embodiments, R₈ is C₁-C₆ alkyl. In some embodiments, R₈ is methyl. In some embodiments, R₈ is ethyl. In some embodiments, R₈ is propyl. In some embodiments, R₈ is butyl. In some embodiments, R₈ is pentyl. In some embodiments, R₈ is hexyl.

In some embodiments, R₇ and R₈ are different.

In some embodiments, R₇ and R₈ are the same.

In some embodiments, R₇ and R₈ are each methyl.

In some embodiments, R₇ and R₈, together with the carbon atom they are attached, form a C₃-C₆ cycloalkyl optionally substituted with one or more OH, halogen, or C₁-C₆ alkoxyl. In some embodiments, R₇ and R₈, together with the carbon atom they are attached, form a cyclopropyl optionally substituted with one or more OH, halogen, or C₁-C₆ alkoxyl. In some embodiments, R₇ and R₈, together with the carbon atom they are attached, form a cyclobutyl optionally substituted with one or more OH, halogen, or C₁-C₆ alkoxyl. In some embodiments, R₇ and R₈, together with the carbon atom they are attached, form a cyclopentyl optionally substituted with one or more OH, halogen, or C₁-C₆ alkoxyl. In some embodiments, R₇ and R₈, together with the carbon atom they are attached, form a cyclohexyl optionally substituted with one or more OH, halogen, or C₁-C₆ alkoxyl.

In some embodiments, R₇ and R₈, together with the carbon atom they are attached, form a C₃-C₆ cycloalkyl. In some embodiments, R₇ and R₈, together with the carbon atom they are attached, form a cyclopropyl. In some embodiments, R₇ and R₈, together with the carbon atom they are attached, form a cyclobutyl. In some embodiments, R₇ and R₈, together with the carbon atom they are attached, form a cyclopentyl. In some embodiments, R₇ and R₈, together with the carbon atom they are attached, form a cyclohexyl.

In some embodiments, m is 1, n is 1, and p is 1.

In some embodiments, m is 1, n is 1, p is 1, and R₂ is C₆-C₁₀ aryl.

In some embodiments, m is 1, n is 1, p is 1, and R₂ is phenyl.

In some embodiments, m is 1, n is 1, p is 1, and R₂ is a 5- to 14-membered saturated or unsaturated heterocycloalkyl comprising 1-5 heteroatoms selected from N, O, and S.

In some embodiments, m is 1, n is 2, and p is 1.

In some embodiments, m is 1, n is 2, p is 1, and R₂ is C₆-C₁₀ aryl.

In some embodiments, m is 1, n is 2, p is 1, and R₂ is phenyl.

In some embodiments, m is 1, n is 2, p is 1, and R₂ is a 5- to 14-membered saturated or unsaturated heterocycloalkyl comprising 1-5 heteroatoms selected from N, O, and S

In some embodiments, m is 1, n is 1, p is 1, X₁ is —C(O)O— and X₂ is absent.

In some embodiments, m is 1, n is 1, p is 1, X₁ is absent, and X₂ is absent.

In some embodiments, m is 1, n is 1, p is 1, X₁ is —O—, and X₂ is absent.

In some embodiments, m is 1, n is 1, p is 1, X₁ is —S(O)₂—, and X₂ is absent.

In some embodiments, m is 1, n is 1, p is 1, X₁ is —C(O) NR₃— and X₂ is absent.

In some embodiments, m is 1, n is 2, p is 1, X₁ is absent, and X₂ is —O—.

In some embodiments, m is 1, n is 2, p is 1, X₁ is absent, and X₂ is S(O)₂.

In some embodiments, m is 1, n is 2, p is 1, X₁ is absent, and X₂ is —C(O) NR₃—.

In some aspects, the present disclosure provides, inter alia, a compound of Formula (I) that is a compound of Formula (I′):

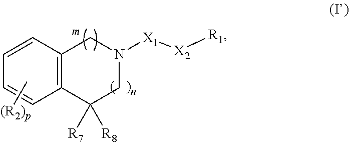

or a prodrug, solvate, or pharmaceutically acceptable salt thereof, wherein:
X₁ is absent or —(CR₃R₄)_{q}—;
X₂ is absent, —S(O)₂—, —C(O)—, or —C(O)O—;
R₁ is C₁-C₆ alkyl, C₃-C₆ cycloalkyl, C₆-C₁₀ aryl, 5- to 14-membered saturated or unsaturated heterocycloalkyl comprising 1-5 heteroatoms selected from N, O, and S; wherein the alkyl, cycloalkyl, aryl, or heterocycloalkyl of any of the foregoing is optionally substituted with one or two R₅;
each R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is optionally substituted with one or two R₆;
each R₃ is H;
each R₄ is H;
each R₅ independently is halogen or C₁-C₆ alkyl;
each R₆ independently is C₁-C₆ alkyl, C₁-C₆ haloalkyl, C₃-C₆ cycloalkyl, C₆-C₁₀ aryl, or 5- to 14-membered saturated or unsaturated heterocycloalkyl comprising 1-5 heteroatoms selected from N, O, and S;
R₇ is C₁-C₆ alkyl;
R₈ is C₁-C₆ alkyl;
m is 1;
n is 1 or 2;
p is 1; and
q is 2.

In some aspects, the compound of Formula (I′) is a compound of Formula (I″), or a prodrug, solvate, or pharmaceutically acceptable salt thereof, wherein:
X₁ is absent;
X₂ is —S(O)₂— or —C(O)O—;
R₁ is C₁-C₆ alkyl, C₃-C₆ cycloalkyl, or C₆-C₁₀ aryl; wherein the alkyl, cycloalkyl, or aryl of any of the foregoing is optionally substituted with one or two R₅;
each R₂ is 5- to 14-membered saturated or unsaturated heterocycloalkyl comprising 1-5 heteroatoms selected from N, O, and S that is optionally substituted with one or two R₆;
each R₅ independently is halogen;
each R₆ independently is C₃-C₆ cycloalkyl;
R₇ is C₁-C₆ alkyl;
R₈ is C₁-C₆ alkyl;
m is 1;
nis 1; and
p is 1.

In some aspects, the compound of Formula (I′) is a compound of Formula (I′″), or a prodrug, solvate, or pharmaceutically acceptable salt thereof, wherein
X₁ is absent or —(CR₃R₄)_{q}—;
X₂ is absent, —S(O)₂—, —C(O)—, or —C(O)O—;
R₁ is C₁-C₆ alkyl, C₃-C₆ cycloalkyl, C₆-C₁₀ aryl, 5- to 14-membered saturated or unsaturated heterocycloalkyl comprising 1-5 heteroatoms selected from N, O, and S; wherein the alkyl, cycloalkyl, aryl, or heterocycloalkyl of any of the foregoing is optionally substituted with one or two R₅;
each R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is optionally substituted with one or two R₆;
each R₃ is H;
each R₄ is H;
each R₅ independently is halogen or C₁-C₆ alkyl;
each R₆ independently is C₁-C₆ alkyl, C₁-C₆ haloalkyl, C₃-C₆ cycloalkyl, C₆-C₁₀ aryl, or 5- to 14-membered saturated or unsaturated heterocycloalkyl comprising 1-5 heteroatoms selected from N, O, and S;
R₇ is C₁-C₆ alkyl;
R₈ is C₁-C₆ alkyl;
m is 1;
n is 2;
p is 1; and
q is 2.

In some embodiments, the compound of Formula (I) is a compound of Formula (II)

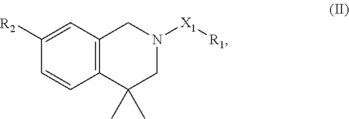

or a prodrug, solvate, or pharmaceutically acceptable salt thereof, wherein X₁, R₁, R₂, R₃, R₄, and R₅ are as described herein for Formula (I).

In some embodiments, the compound of Formula (I) is a compound of Formula (IIa):

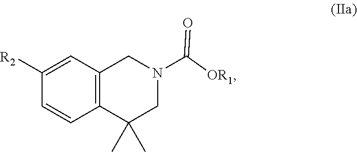

or a prodrug, solvate, or pharmaceutically acceptable salt thereof, wherein R₁, R₂, and R₅ are as described herein for Formula (I).

In some embodiments, the compound of Formula (I) is a compound of Formula (IIb):

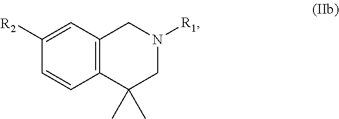

or a prodrug, solvate, or pharmaceutically acceptable salt thereof, wherein R₁, R₂, and R₅ are as described herein for Formula (I).

In some embodiments, the compound of Formula (I) is a compound of Formula (IIc):

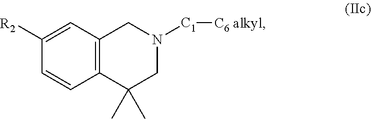

or a prodrug, solvate, or pharmaceutically acceptable salt thereof, wherein R₂ and R₅ are as described herein for Formula (I).

In some embodiments, the compound of Formula (I) is a compound of Formula (IId):

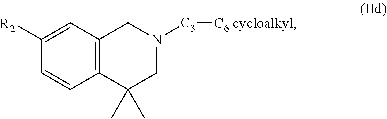

or a prodrug, solvate, or pharmaceutically acceptable salt thereof, wherein R₂ and R₅ are as described herein for Formula (I).

In some embodiments, the compound of Formula (I) is a compound of Formula (IIe):

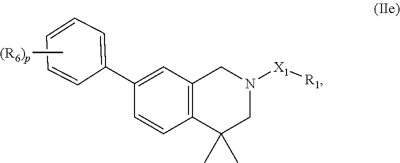

or a prodrug, solvate, or pharmaceutically acceptable salt thereof, wherein X₁, R₁, R₃, R₄, R₆, and p are as described herein for Formula (I).

In some embodiments, the compound of Formula (I) is a compound of Formula (IIf):

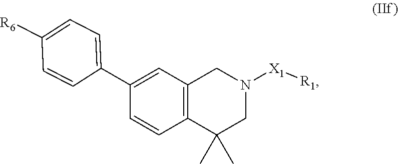

or a prodrug, solvate, or pharmaceutically acceptable salt thereof, wherein X₁, R₁, R₃, R₄, and R₆ are as described herein for Formula (I).

In some embodiments, the compound of Formula (I) is a compound of Formula (IIg):

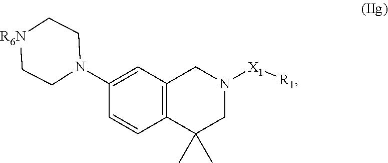

or a prodrug, solvate, or pharmaceutically acceptable salt thereof, wherein X₁, R₁, R₃, R₄, and R₆ are as described herein for Formula (I).

In some embodiments, the compound of Formula (I) is a compound of Formula (IIh):

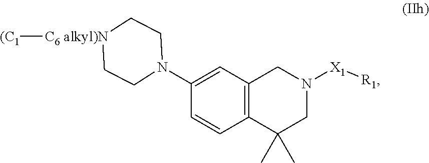

or a prodrug, solvate, or pharmaceutically acceptable salt thereof, wherein X₁, R₁, R₃, and R₄ are as described herein for Formula (I).

In some embodiments, the compound of Formula (I) is a compound of Formula (IIi):

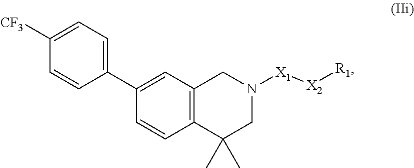

or a prodrug, solvate, or pharmaceutically acceptable salt thereof, wherein X₁, X₂, R₁, R₃, R₄, and R₅ are as described herein for Formula (I).

In some embodiments, the compound of Formula (I) is a compound of Formula (IIj):

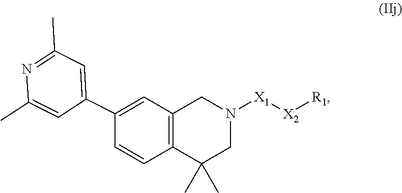

or a prodrug, solvate, or pharmaceutically acceptable salt thereof, wherein X₁, X₂, R₁, R₃, R₄, and R₅ are as described herein for Formula (I).

In some embodiments, the compound of Formula (I) is a compound of Formula (IIk):

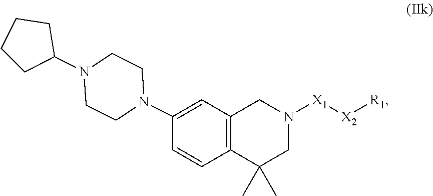

or a prodrug, solvate, or pharmaceutically acceptable salt thereof, wherein X₁, X₂, R₁, R₃, R₄, and R₅ are as described herein for Formula (I).

In some embodiments, the compound of Formula (I) is a compound of Formula (III):

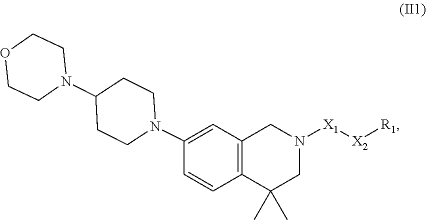

or a prodrug, solvate, or pharmaceutically acceptable salt thereof, wherein X₁, X₂, R₁, R₃, R₄, and R₅ are as described herein for Formula (I).

In some embodiments, the compound of Formula (I) is a compound of Formula (III):

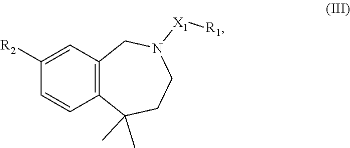

or a prodrug, solvate, or pharmaceutically acceptable salt thereof, wherein X₁, R₁, R₂, R₃, R₄, and R₅ are as described herein for Formula (I).

In some embodiments, the compound of Formula (I) is a compound of Formula (IIIa):

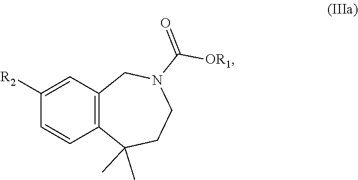

or a prodrug, solvate, or pharmaceutically acceptable salt thereof, wherein R₁, R₂, and R₅ are as described herein for Formula (I).

In some embodiments, the compound of Formula (I) is a compound of Formula (IIIb):

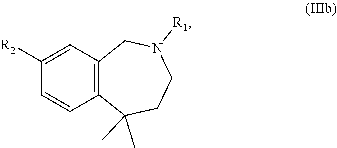

or a prodrug, solvate, or pharmaceutically acceptable salt thereof, wherein R₁, R₂, and R₅ are as described herein for Formula (I).

In some embodiments, the compound of Formula (I) is a compound of Formula (IIIc):

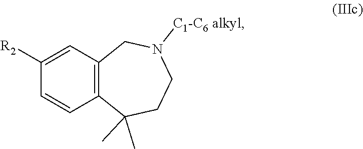

or a prodrug, solvate, or pharmaceutically acceptable salt thereof, wherein R₂ and R₅ are as described herein for Formula (I).

In some embodiments, the compound of Formula (I) is a compound of Formula (IIId):

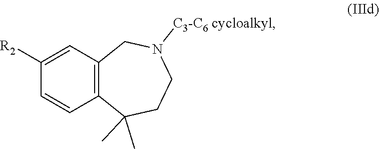

or a prodrug, solvate, or pharmaceutically acceptable salt thereof, wherein R₂ and R₅ are as described herein for Formula (I).

In some embodiments, the compound of Formula (I) is a compound of Formula (IIIe):

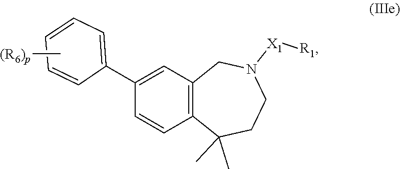

or a prodrug, solvate, or pharmaceutically acceptable salt thereof, wherein X₁, R₁, R₃, R₄, R₆, and p are as described herein for Formula (I).

In some embodiments, the compound of Formula (I) is a compound of Formula (IIIf):

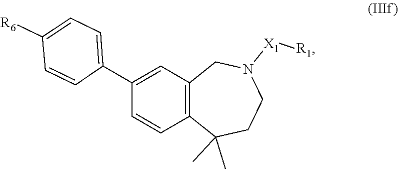

or a prodrug, solvate, or pharmaceutically acceptable salt thereof, wherein X₁, R₁, R₃, R₄, and R₆ are as described herein for Formula (I).

In some embodiments, the compound of Formula (I) is a compound of Formula (IIIg):

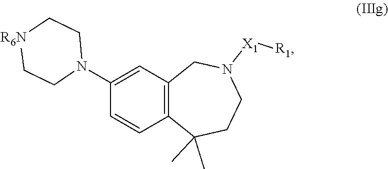

or a prodrug, solvate, or pharmaceutically acceptable salt thereof, wherein X₁, R₁, R₃, R₄, and R₆ are as described herein for Formula (I).

In some embodiments, the compound of Formula (I) is a compound of Formula (IIIh):

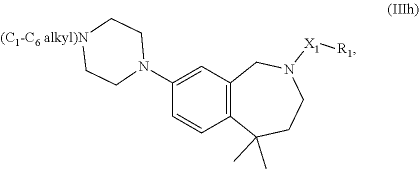

or a prodrug, solvate, or pharmaceutically acceptable salt thereof, wherein X₁, R₁, R₃, and R₄ are as described herein for Formula (I).

In some embodiments, the compound of Formula (I) is a compound of Formula (IIIi):

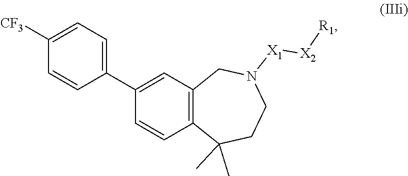

or a prodrug, solvate, or pharmaceutically acceptable salt thereof, wherein X₁, X₂, R₁, R₃, R₄, and R₅ are as described herein for Formula (I).

In some embodiments, the compound of Formula (I) is a compound of Formula (IIIj):

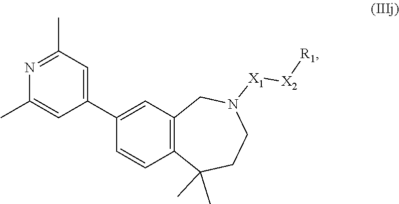

or a prodrug, solvate, or pharmaceutically acceptable salt thereof, wherein X₁, X₂, R₁, R₃, R₄, and R₅ are as described herein for Formula (I).

In some embodiments, the compound of Formula (I) is a compound of Formula (IIIk):

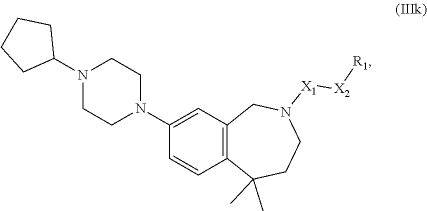

or a prodrug, solvate, or pharmaceutically acceptable salt thereof, wherein X₁, X₂, R₁, R₃, R₄, and R₅ are as described herein for Formula (I).

In some embodiments, the compound of Formula (I) is a compound of Formula (IIII):

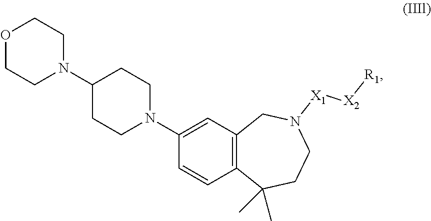

or a prodrug, solvate, or pharmaceutically acceptable salt thereof, wherein X₁, X₂, R₁, R₃, R₄, and R₅ are as described herein for Formula (I).

In some aspects, the present disclosure provides a compound, 2-(2-(1-(2-(4-fluorophenyl)-2-oxoethyl) pyrrolidin-3-yl)ethyl) isoindolin-1-one, i.e., MIN-101-B:

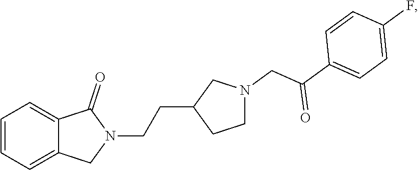 [SMILES: O=C(CN1CCC(CCN2Cc3ccccc3C2=O)C1)c1ccc(F)cc1]

or a prodrug, solvate, or pharmaceutically acceptable salt thereof.

In some embodiments, MIN-101-B is(S)-2-(2-(1-(2-(4-fluorophenyl)-2-oxoethyl) pyrrolidin-3-yl)ethyl) isoindolin-1-one,

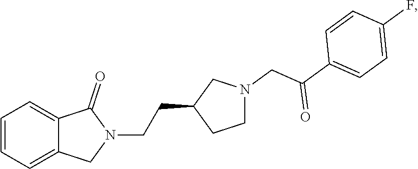 [SMILES: O=C(CN1CC[C@@H](CCN2Cc3ccccc3C2=O)C1)c1ccc(F)cc1]

or a prodrug, solvate, or pharmaceutically acceptable salt thereof.

In some embodiments, MIN-101-B is (R)-2-(2-(1-(2-(4-fluorophenyl)-2-oxoethyl) pyrrolidin-3-yl)ethyl) isoindolin-1-one,

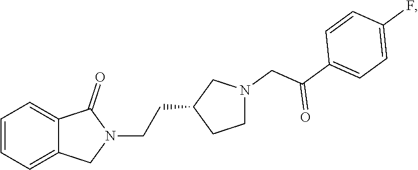 [SMILES: O=C(CN1CC[C@H](CCN2Cc3ccccc3C2=O)C1)c1ccc(F)cc1]

or a prodrug, solvate, or pharmaceutically acceptable salt thereof.

In some embodiments, the compound of the present disclosure is selected from the compounds described below in Table 1.

TABLE 1

| Compound | |
|---|---|
| No. | Structure and name |
| 1 | 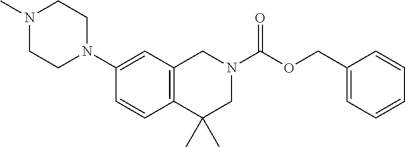 [SMILES: CN1CCN(c2ccc3c(c2)CN(C(=O)OCc2ccccc2)CC3(C)C)CC1] |
| | benzyl 4,4-dimethyl-7-(4-methylpiperazin-1-yl)- |
| | 3,4-dihydroisoquinoline-2(1H)-carboxylate |
| 2 | 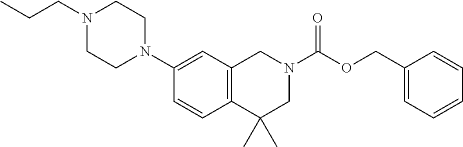 [SMILES: CCCN1CCN(c2ccc3c(c2)CN(C(=O)OCc2ccccc2)CC3(C)C)CC1] |
| | benzyl 4,4-dimethyl-7-(4-propylpiperazin-1-yl)- |
| | 3,4-dihydroisoquinoline-2(1H)-carboxylate |
| 3 | 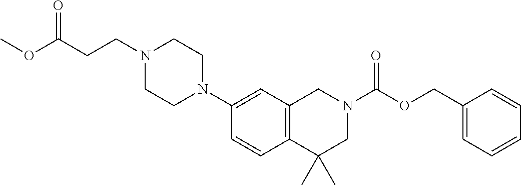 [SMILES: COC(=O)CCN1CCN(c2ccc3c(c2)CN(C(=O)OCc2ccccc2)CC3(C)C)CC1] |
| | benzyl 7-(4-(3-methoxy-3-oxopropyl)piperazin-1- |
| | yl)-4,4-dimethyl-3,4-dihydroisoquinoline-2(1H)- |
| | carboxylate |
| 4 | 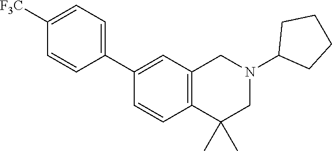 [SMILES: CC1(C)CN(C2CCCC2)Cc2cc(-c3ccc(C(F)(F)F)cc3)ccc21] |
| | 2-cyclopentyl-4,4-dimethyl-7-(4- |
| | (trifluoromethyl)phenyl)-1,2,3,4- |
| | tetrahydroisoquinoline |
| 5 | 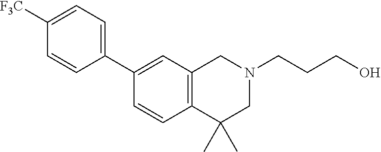 [SMILES: CC1(C)CN(CCCO)Cc2cc(-c3ccc(C(F)(F)F)cc3)ccc21] |
| | 3-(4,4-dimethyl-7-(4-(trifluoromethyl)phenyl)-3,4- |
| | dihydroisoquinolin-2(1H)-yl)propan-1-ol |
| 6 | 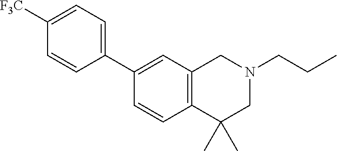 [SMILES: CCCN1Cc2cc(-c3ccc(C(F)(F)F)cc3)ccc2C(C)(C)C1] |
| | 4,4-dimethyl-2-propyl-7-(4- |
| | (trifluoromethyl)phenyl)-1,2,3,4- |
| | tetrahydroisoquinoline |
| 7 | 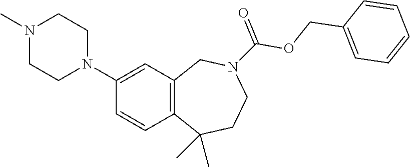 [SMILES: CN1CCN(c2ccc3c(c2)CN(C(=O)OCc2ccccc2)CCC3(C)C)CC1] |
| | benzyl 5,5-dimethyl-8-(4-methylpiperazin-1-yl)- |
| | 1,3,4,5-tetrahydro-2H-benzo[c]azepine-2- |
| | carboxylate |
| 8 | 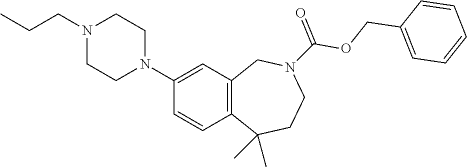 [SMILES: CCCN1CCN(c2ccc3c(c2)CN(C(=O)OCc2ccccc2)CCC3(C)C)CC1] |
| | benzyl 5,5-dimethyl-8-(4-propylpiperazin-1-yl)- |
| | 1,3,4,5-tetrahydro-2H-benzo[c]azepine-2- |
| | carboxylate |
| 9 | 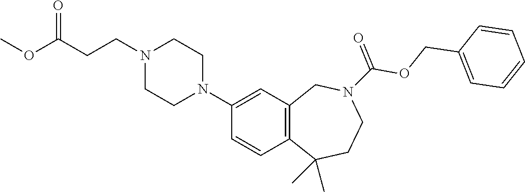 [SMILES: COC(=O)CCN1CCN(c2ccc3c(c2)CN(C(=O)OCc2ccccc2)CCC3(C)C)CC1] |
| | benzyl 8-(4-(3-methoxy-3-oxopropyl)piperazin-1- |
| | yl)-5,5-dimethyl-1,3,4,5-tetrahydro-2H- |
| | benzo[c]azepine-2-carboxylate |
| 10 | 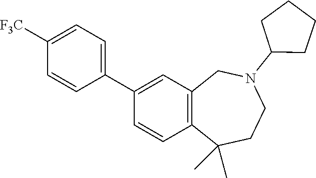 [SMILES: CC1(C)CCN(C2CCCC2)Cc2cc(-c3ccc(C(F)(F)F)cc3)ccc21] |
| | 2-cyclopentyl-5,5-dimethyl-8-(4- |
| | (trifluoromethyl)phenyl)-2,3,4,5-tetrahydro-1H- |
| | benzo[c]azepine |
| 11 | 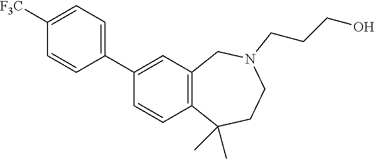 [SMILES: CC1(C)CCN(CCCO)Cc2cc(-c3ccc(C(F)(F)F)cc3)ccc21] |
| | 3-(5,5-dimethyl-8-(4-(trifluoromethyl)phenyl)- |
| | 1,3,4,5-tetrahydro-2H-benzo[c]azepin-2-yl)propan- |
| | 1-ol |
| 12 | 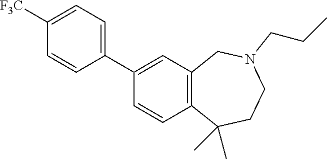 [SMILES: CCCN1CCC(C)(C)c2ccc(-c3ccc(C(F)(F)F)cc3)cc2C1] |
| | 5,5-dimethyl-2-propyl-8-(4- |
| | (trifluoromethyl)phenyl)-2,3,4,5-tetrahydro-1H- |
| | benzo[c]azepine |
| 13 | 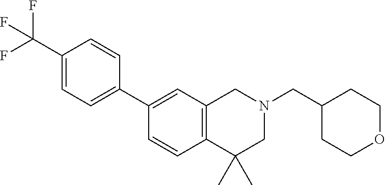 [SMILES: CC1(C)CN(CC2CCOCC2)Cc2cc(-c3ccc(C(F)(F)F)cc3)ccc21] |
| | 4,4-dimethyl-2-((tetrahydro-2H-pyran-4- |
| | yl)methyl)-7-(4-(trifluoromethyl)phenyl)-1,2,3,4- |
| | tetrahydroisoquinoline |
| 14 | 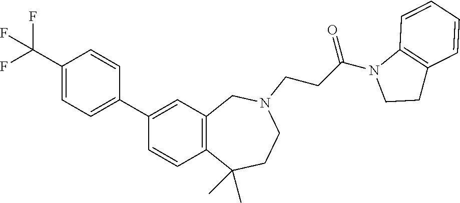 [SMILES: CC1(C)CCN(CCC(=O)N2CCc3ccccc32)Cc2cc(-c3ccc(C(F)(F)F)cc3)ccc21] |
| | 3-(5,5-dimethyl-8-(4-(trifluoromethyl)phenyl)- |
| | 1,3,4,5-tetrahydro-2H-benzo[c]azepin-2-yl)-1- |
| | (indolin-1-yl)propan-1-one |
| 15 | 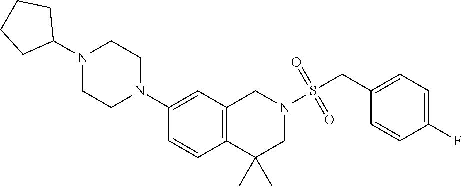 [SMILES: CC1(C)CN(S(=O)(=O)Cc2ccc(F)cc2)Cc2cc(N3CCN(C4CCCC4)CC3)ccc21] |
| | 7-(4-cyclopentylpiperazin-1-yl)-2-((4- |
| | fluorobenzyl)sulfonyl)-4,4-dimethyl-1,2,3,4- |
| | tetrahydroisoquinoline |
| 16 | 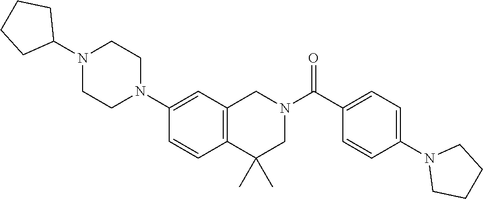 [SMILES: CC1(C)CN(C(=O)c2ccc(N3CCCC3)cc2)Cc2cc(N3CCN(C4CCCC4)CC3)ccc21] |
| | (7-(4-cyclopentylpiperazin-1-yl)-4,4-dimethyl-3,4- |
| | dihydroisoquinolin-2(1H)-yl)(4-(pyrrolidin-1- |
| | yl)phenyl)methanone |
| 17 | 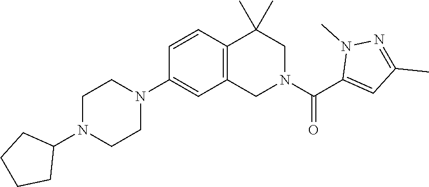 [SMILES: Cc1cc(C(=O)N2Cc3cc(N4CCN(C5CCCC5)CC4)ccc3C(C)(C)C2)n(C)n1] |
| | (7-(4-cyclopentylpiperazin-1-yl)-4,4-dimethyl-3,4- |
| | dihydroisoquinolin-2(1H)-yl)(1,3-dimethyl-1H- |
| | pyrazol-5-yl)methanone |
| 18 | 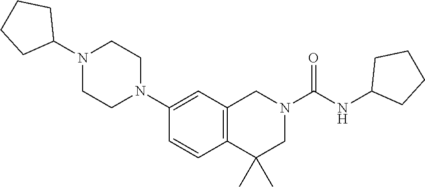 [SMILES: CC1(C)CN(C(=O)NC2CCCC2)Cc2cc(N3CCN(C4CCCC4)CC3)ccc21] |
| | N-cyclopentyl-7-(4-cyclopentylpiperazin-1-yl)-4,4- |
| | dimethyl-3,4-dihydroisoquinoline-2(1H)- |
| | carboxamide |
| 19 | 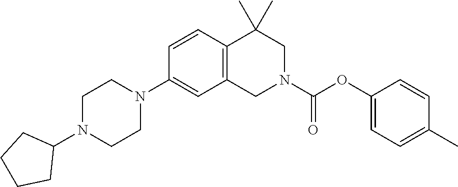 [SMILES: Cc1ccc(OC(=O)N2Cc3cc(N4CCN(C5CCCC5)CC4)ccc3C(C)(C)C2)cc1] |
| | p-tolyl 7-(4-cyclopentylpiperazin-1-yl)-4,4- |
| | dimethyl-3,4-dihydroisoquinoline-2(1H)- |
| | carboxylate |
| 20 | 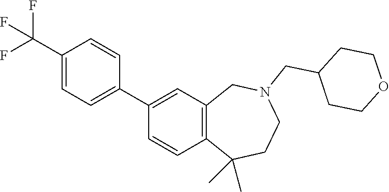 [SMILES: CC1(C)CCN(CC2CCOCC2)Cc2cc(-c3ccc(C(F)(F)F)cc3)ccc21] |
| | 5,5-dimethyl-2-((tetrahydro-2H-pyran-4- |
| | yl)methyl)-8-(4-(trifluoromethyl)phenyl)-2,3,4,5- |
| | tetrahydro-1H-benzo[c]azepine |
| 21 | 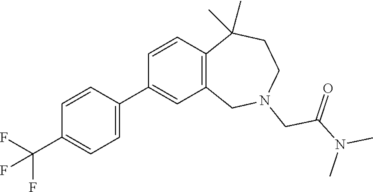 [SMILES: CN(C)C(=O)CN1CCC(C)(C)c2ccc(-c3ccc(C(F)(F)F)cc3)cc2C1] |
| | 2-(5,5-dimethyl-8-(4-(trifluoromethyl)phenyl)- |
| | 1,3,4,5-tetrahydro-2H-benzo[c]azepin-2-yl)-N,N- |
| | dimethylacetamide |
| 22 | 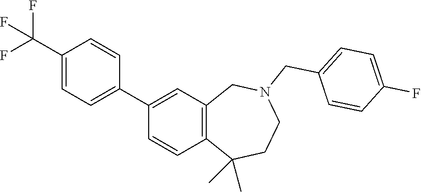 [SMILES: CC1(C)CCN(Cc2ccc(F)cc2)Cc2cc(-c3ccc(C(F)(F)F)cc3)ccc21] |
| | 2-(4-fluorobenzyl)-5,5-dimethyl-8-(4- |
| | (trifluoromethyl)phenyl)-2,3,4,5-tetrahydro-1H- |
| | benzo[c]azepine |
| 23 | 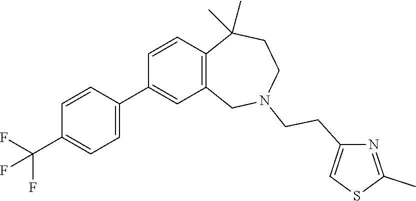 [SMILES: Cc1nc(CCN2CCC(C)(C)c3ccc(-c4ccc(C(F)(F)F)cc4)cc3C2)cs1] |
| | 4-(2-(5,5-dimethyl-8-(4-(trifluoromethyl)phenyl)- |
| | 1,3,4,5-tetrahydro-2H-benzo[c]azepin-2-yl)ethyl)- |
| | 2-methylthiazole |
| 24 | 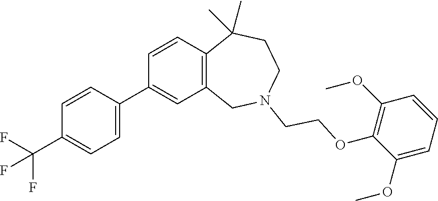 [SMILES: COc1cccc(OC)c1OCCN1CCC(C)(C)c2ccc(-c3ccc(C(F)(F)F)cc3)cc2C1] |
| | 2-(2-(2,6-dimethoxyphenoxy)ethyl)-5,5-dimethyl- |
| | 8-(4-(trifluoromethyl)phenyl)-2,3,4,5-tetrahydro- |
| | 1H-benzo[c]azepine |
| 25 | 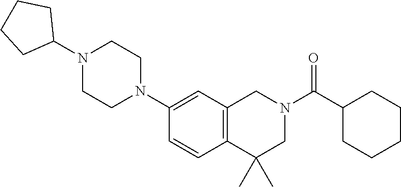 [SMILES: CC1(C)CN(C(=O)C2CCCCC2)Cc2cc(N3CCN(C4CCCC4)CC3)ccc21] |
| | cyclohexyl(7-(4-cyclopentylpiperazin-1-yl)-4,4- |
| | dimethyl-3,4-dihydroisoquinolin-2(1H)- |
| | yl)methanone |
| 26 | 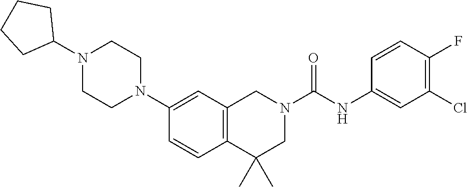 [SMILES: CC1(C)CN(C(=O)Nc2ccc(F)c(Cl)c2)Cc2cc(N3CCN(C4CCCC4)CC3)ccc21] |
| | N-(3-chloro-4-fluorophenyl)-7-(4- |
| | cyclopentylpiperazin-1-yl)-4,4-dimethyl-3,4- |
| | dihydroisoquinoline-2(1H)-carboxamide |
| 27 | 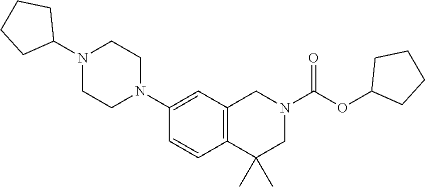 [SMILES: CC1(C)CN(C(=O)OC2CCCC2)Cc2cc(N3CCN(C4CCCC4)CC3)ccc21] |
| | cyclopentyl 7-(4-cyclopentylpiperazin-1-yl)-4,4- |
| | dimethyl-3,4-dihydroisoquinoline-2(1H)- |
| | carboxylate |
| 28 | 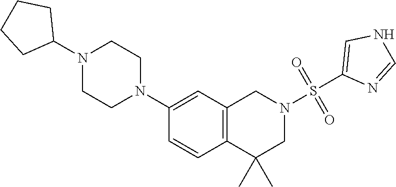 [SMILES: CC1(C)CN(S(=O)(=O)c2c[nH]cn2)Cc2cc(N3CCN(C4CCCC4)CC3)ccc21] |
| | 2-((1H-imidazol-4-yl)sulfonyl)-7-(4- |
| | cyclopentylpiperazin-1-yl)-4,4-dimethyl-1,2,3,4- |
| | tetrahydroisoquinoline |
| 29 | 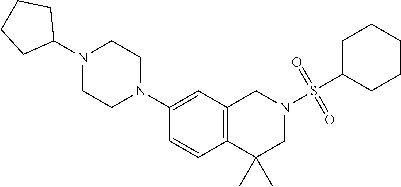 [SMILES: CC1(C)CN(S(=O)(=O)C2CCCCC2)Cc2cc(N3CCN(C4CCCC4)CC3)ccc21] |
| | 2-(cyclohexylsulfonyl)-7-(4-cyclopentylpiperazin- |
| | 1-yl)-4,4-dimethyl-1,2,3,4-tetrahydroisoquinoline |
| 30 | 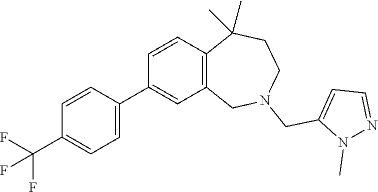 [SMILES: Cn1nccc1CN1CCC(C)(C)c2ccc(-c3ccc(C(F)(F)F)cc3)cc2C1] |
| | 5,5-dimethyl-2-((1-methyl-1H-pyrazol-5- |
| | yl)methyl)-8-(4-(trifluoromethyl)phenyl)-2,3,4,5- |
| | tetrahydro-1H-benzo[c]azepine |
| 31 | 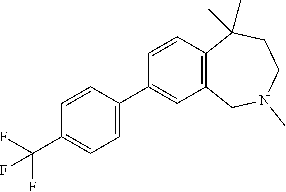 [SMILES: CN1CCC(C)(C)c2ccc(-c3ccc(C(F)(F)F)cc3)cc2C1] |
| | 2,5,5-trimethyl-8-(4-(trifluoromethyl)phenyl)- |
| | 2,3,4,5-tetrahydro-1H-benzo[c]azepine |
| 32 | 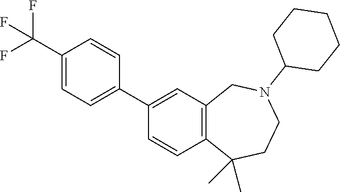 [SMILES: CC1(C)CCN(C2CCCCC2)Cc2cc(-c3ccc(C(F)(F)F)cc3)ccc21] |
| | 2-cyclohexyl-5,5-dimethyl-8-(4- |
| | (trifluoromethyl)phenyl)-2,3,4,5-tetrahydro-1H- |
| | benzo[c]azepine |
| 33 | 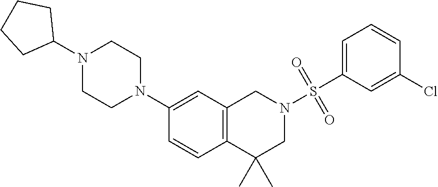 [SMILES: CC1(C)CN(S(=O)(=O)c2cccc(Cl)c2)Cc2cc(N3CCN(C4CCCC4)CC3)ccc21] |
| | 2-((3-chlorophenyl)sulfonyl)-7-(4- |
| | cyclopentylpiperazin-1-yl)-4,4-dimethyl-1,2,3,4- |
| | tetrahydroisoquinoline |
| 34 | 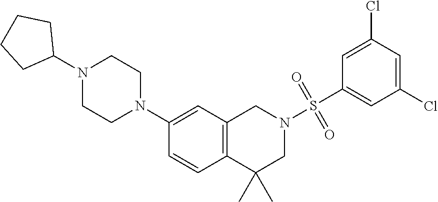 [SMILES: CC1(C)CN(S(=O)(=O)c2cc(Cl)cc(Cl)c2)Cc2cc(N3CCN(C4CCCC4)CC3)ccc21] |
| | 7-(4-cyclopentylpiperazin-1-yl)-2-((3,5- |
| | dichlorophenyl)sulfonyl)-4,4-dimethyl-1,2,3,4- |
| | tetrahydroisoquinoline |
| 35 | 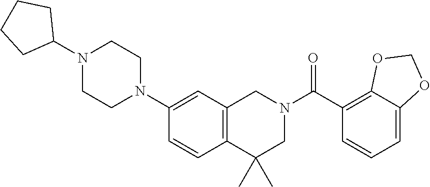 [SMILES: CC1(C)CN(C(=O)c2cccc3c2OCO3)Cc2cc(N3CCN(C4CCCC4)CC3)ccc21] |
| | benzo[d][1,3]dioxol-4-yl(7-(4- |
| | cyclopentylpiperazin-1-yl)-4,4-dimethyl-3,4- |
| | dihydroisoquinolin-2(1H)-yl)methanone |
| 36 | 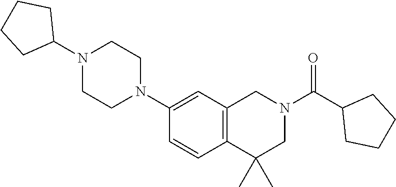 [SMILES: CC1(C)CN(C(=O)C2CCCC2)Cc2cc(N3CCN(C4CCCC4)CC3)ccc21] |
| | cyclopentyl(7-(4-cyclopentylpiperazin-1-yl)-4,4- |
| | dimethyl-3,4-dihydroisoquinolin-2(1H)- |
| | yl)methanone |
| 37 | 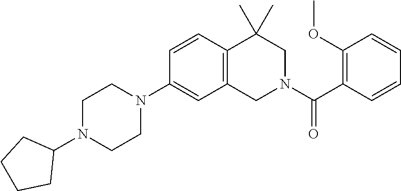 [SMILES: COc1ccccc1C(=O)N1Cc2cc(N3CCN(C4CCCC4)CC3)ccc2C(C)(C)C1] |
| | (7-(4-cyclopentylpiperazin-1-yl)-4,4-dimethyl-3,4- |
| | dihydroisoquinolin-2(1H)-yl)(2- |
| | methoxyphenyl)methanone |
| 38 | 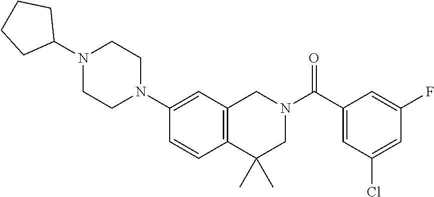 [SMILES: CC1(C)CN(C(=O)c2cc(F)cc(Cl)c2)Cc2cc(N3CCN(C4CCCC4)CC3)ccc21] |
| | (3-chloro-5-fluorophenyl)(7-(4- |
| | cyclopentylpiperazin-1-yl)-4,4-dimethyl-3,4- |
| | dihydroisoquinolin-2(1H)-yl)methanone |
| 39 | 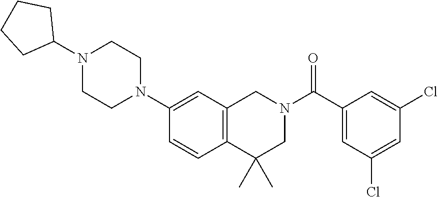 [SMILES: CC1(C)CN(C(=O)c2cc(Cl)cc(Cl)c2)Cc2cc(N3CCN(C4CCCC4)CC3)ccc21] |
| | (7-(4-cyclopentylpiperazin-1-yl)-4,4-dimethyl-3,4- |
| | dihydroisoquinolin-2(1H)-yl)(3,5- |
| | dichlorophenyl)methanone |
| 40 | 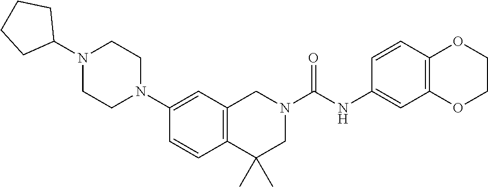 [SMILES: CC1(C)CN(C(=O)Nc2ccc3c(c2)OCCO3)Cc2cc(N3CCN(C4CCCC4)CC3)ccc21] |
| | 7-(4-cyclopentylpiperazin-1-yl)-N-(2,3- |
| | dihydrobenzo[b][1,4]dioxin-6-yl)-4,4-dimethyl-3,4- |
| | dihydroisoquinoline-2(1H)-carboxamide |
| 41 | 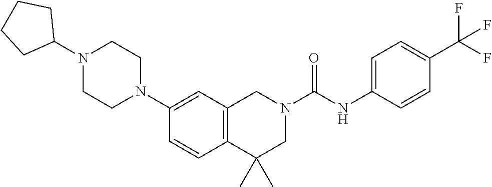 [SMILES: CC1(C)CN(C(=O)Nc2ccc(C(F)(F)F)cc2)Cc2cc(N3CCN(C4CCCC4)CC3)ccc21] |
| | 7-(4-cyclopentylpiperazin-1-yl)-4,4-dimethyl-N-(4- |
| | (trifluoromethyl)phenyl)-3,4-dihydroisoquinoline- |
| | 2(1H)-carboxamide |
| 42 | 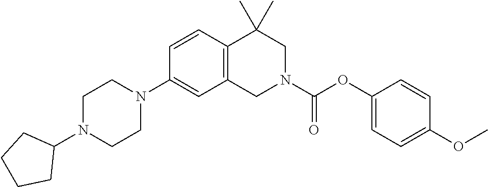 [SMILES: COc1ccc(OC(=O)N2Cc3cc(N4CCN(C5CCCC5)CC4)ccc3C(C)(C)C2)cc1] |
| | 4-methoxyphenyl 7-(4-cyclopentylpiperazin-1-yl)- |
| | 4,4-dimethyl-3,4-dihydroisoquinoline-2(1H)- |
| | carboxylate |
| 43 | 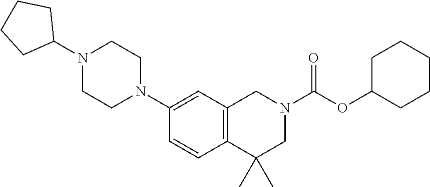 [SMILES: CC1(C)CN(C(=O)OC2CCCCC2)Cc2cc(N3CCN(C4CCCC4)CC3)ccc21] |
| | cyclohexyl 7-(4-cyclopentylpiperazin-1-yl)-4,4- |
| | dimethyl-3,4-dihydroisoquinoline-2(1H)- |
| | carboxylate |
| 44 | 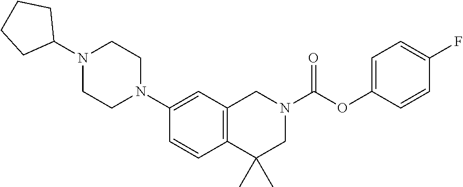 [SMILES: CC1(C)CN(C(=O)Oc2ccc(F)cc2)Cc2cc(N3CCN(C4CCCC4)CC3)ccc21] |
| | 4-fluorophenyl 7-(4-cyclopentylpiperazin-1-yl)-4,4- |
| | dimethyl-3,4-dihydroisoquinoline-2(1H)- |
| | carboxylate |
| 45 | 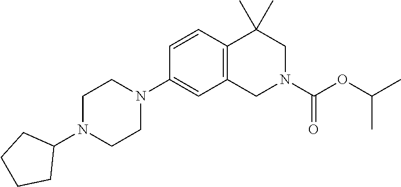 [SMILES: CC(C)OC(=O)N1Cc2cc(N3CCN(C4CCCC4)CC3)ccc2C(C)(C)C1] |
| | isopropyl 7-(4-cyclopentylpiperazin-1-yl)-4,4- |
| | dimethyl-3,4-dihydroisoquinoline-2(1H)- |
| | carboxylate |
| 46 | 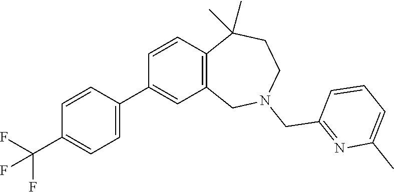 [SMILES: Cc1cccc(CN2CCC(C)(C)c3ccc(-c4ccc(C(F)(F)F)cc4)cc3C2)n1] |
| | 5,5-dimethyl-2-((6-methylpyridin-2-yl)methyl)-8- |
| | (4-(trifluoromethyl)phenyl)-2,3,4,5-tetrahydro-1H- |
| | benzo[c]azepine |
| 47 | 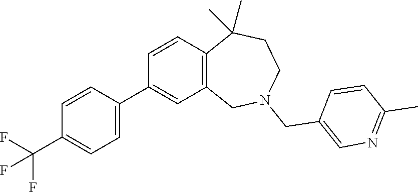 [SMILES: Cc1ccc(CN2CCC(C)(C)c3ccc(-c4ccc(C(F)(F)F)cc4)cc3C2)cn1] |
| | 5,5-dimethyl-2-((6-methylpyridin-3-yl)methyl)-8- |
| | (4-(trifluoromethyl)phenyl)-2,3,4,5-tetrahydro-1H- |
| | benzo[c]azepine |
| 48 | 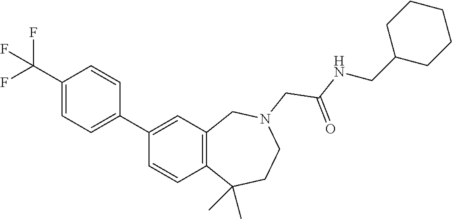 [SMILES: CC1(C)CCN(CC(=O)NCC2CCCCC2)Cc2cc(-c3ccc(C(F)(F)F)cc3)ccc21] |
| | N-(cyclohexylmethyl)-2-(5,5-dimethyl-8-(4- |
| | (trifluoromethyl)phenyl)-1,3,4,5-tetrahydro-2H- |
| | benzo[c]azepin-2-yl)acetamide |
| 49 | 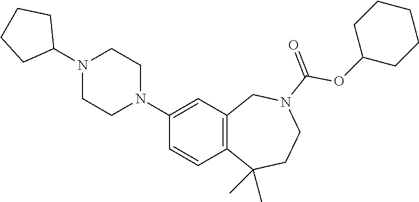 [SMILES: CC1(C)CCN(C(=O)OC2CCCCC2)Cc2cc(N3CCN(C4CCCC4)CC3)ccc21] |
| | cyclohexyl 8-(4-cyclopentylpiperazin-1-yl)-5,5- |
| | dimethyl-1,3,4,5-tetrahydro-2H-benzo[c]azepine-2- |
| | carboxylate |
| 50 | 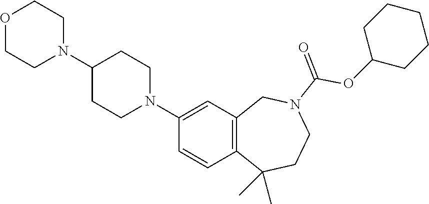 [SMILES: CC1(C)CCN(C(=O)OC2CCCCC2)Cc2cc(N3CCC(N4CCOCC4)CC3)ccc21] |
| | cyclohexyl 5,5-dimethyl-8-(4-morpholinopiperidin- |
| | 1-yl)-1,3,4,5-tetrahydro-2H-benzo[c]azepine-2- |
| | carboxylate |
| 51 | 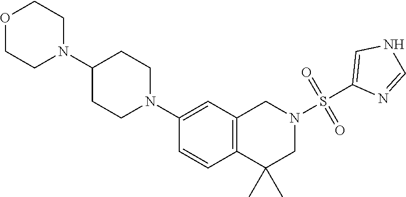 [SMILES: CC1(C)CN(S(=O)(=O)c2c[nH]cn2)Cc2cc(N3CCC(N4CCOCC4)CC3)ccc21] |
| | 4-(1-(2-((1H-imidazol-4-yl)sulfonyl)-4,4-dimethyl- |
| | 1,2,3,4-tetrahydroisoquinolin-7-yl)piperidin-4- |
| | yl)morpholine |
| 52 | 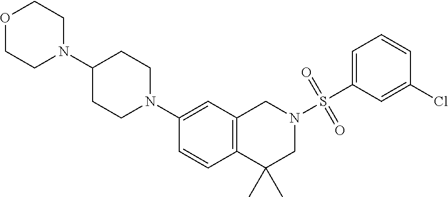 [SMILES: CC1(C)CN(S(=O)(=O)c2cccc(Cl)c2)Cc2cc(N3CCC(N4CCOCC4)CC3)ccc21] |
| | 4-(1-(2-((3-chlorophenyl)sulfonyl)-4,4-dimethyl- |
| | 1,2,3,4-tetrahydroisoquinolin-7-yl)piperidin-4- |
| | yl)morpholine |
| 53 | 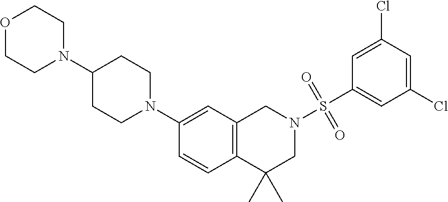 [SMILES: CC1(C)CN(S(=O)(=O)c2cc(Cl)cc(Cl)c2)Cc2cc(N3CCC(N4CCOCC4)CC3)ccc21] |
| | 4-(1-(2-((3,5-dichlorophenyl)sulfonyl)-4,4- |
| | dimethyl-1,2,3,4-tetrahydroisoquinolin-7- |
| | yl)piperidin-4-yl)morpholine |
| 54 | 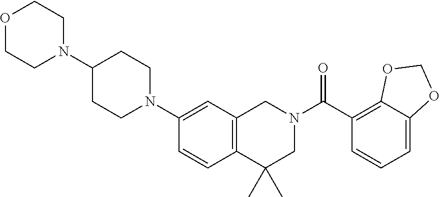 [SMILES: CC1(C)CN(C(=O)c2cccc3c2OCO3)Cc2cc(N3CCC(N4CCOCC4)CC3)ccc21] |
| | benzo[d][1,3]dioxol-4-yl(4,4-dimethyl-7-(4- |
| | morpholinopiperidin-1-yl)-3,4-dihydroisoquinolin- |
| | 2(1H)-yl)methanone |
| 55 | 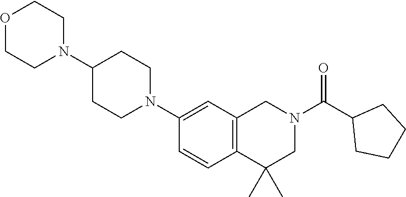 [SMILES: CC1(C)CN(C(=O)C2CCCC2)Cc2cc(N3CCC(N4CCOCC4)CC3)ccc21] |
| | cyclopentyl(4,4-dimethyl-7-(4- |
| | morpholinopiperidin-1-yl)-3,4-dihydroisoquinolin- |
| | 2(1H)-yl)methanone |
| 56 | 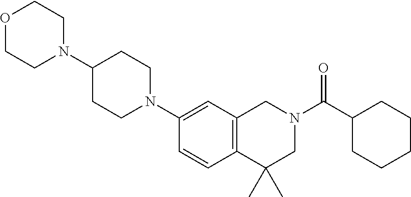 [SMILES: CC1(C)CN(C(=O)C2CCCCC2)Cc2cc(N3CCC(N4CCOCC4)CC3)ccc21] |
| | cyclohexyl(4,4-dimethyl-7-(4-morpholinopiperidin- |
| | 1-yl)-3,4-dihydroisoquinolin-2(1H)-yl)methanone |
| 57 | 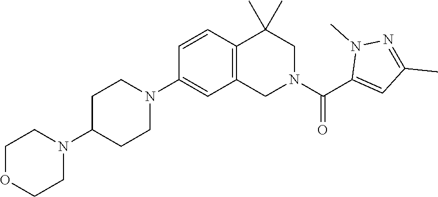 [SMILES: Cc1cc(C(=O)N2Cc3cc(N4CCC(N5CCOCC5)CC4)ccc3C(C)(C)C2)n(C)n1] |
| | (1,3-dimethyl-1H-pyrazol-5-yl)(4,4-dimethyl-7-(4- |
| | morpholinopiperidin-1-yl)-3,4-dihydroisoquinolin- |
| | 2(1H)-yl)methanone |
| 58 | 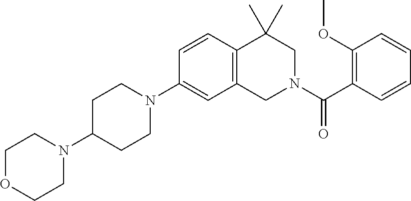 [SMILES: COc1ccccc1C(=O)N1Cc2cc(N3CCC(N4CCOCC4)CC3)ccc2C(C)(C)C1] |
| | (4,4-dimethyl-7-(4-morpholinopiperidin-1-yl)-3,4- |
| | dihydroisoquinolin-2(1H)-yl)(2- |
| | methoxyphenyl)methanone |
| 59 | 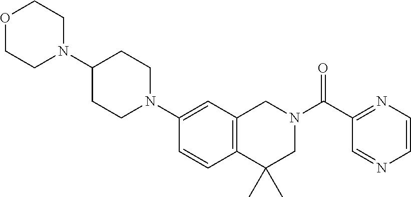 [SMILES: CC1(C)CN(C(=O)c2cnccn2)Cc2cc(N3CCC(N4CCOCC4)CC3)ccc21] |
| | (4,4-dimethyl-7-(4-morpholinopiperidin-1-yl)-3,4- |
| | dihydroisoquinolin-2(1H)-yl)(pyrazin-2- |
| | yl)methanone |
| 60 | 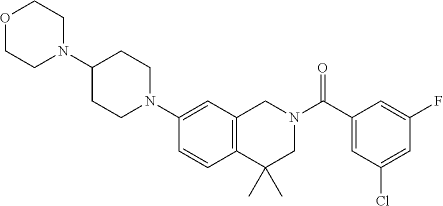 [SMILES: CC1(C)CN(C(=O)c2cc(F)cc(Cl)c2)Cc2cc(N3CCC(N4CCOCC4)CC3)ccc21] |
| | (3-chloro-5-fluorophenyl)(4,4-dimethyl-7-(4- |
| | morpholinopiperidin-1-yl)-3,4-dihydroisoquinolin- |
| | 2(1H)-yl)methanone |
| 61 | 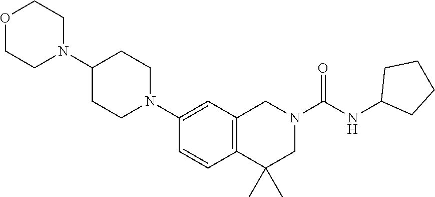 [SMILES: CC1(C)CN(C(=O)NC2CCCC2)Cc2cc(N3CCC(N4CCOCC4)CC3)ccc21] |
| | N-cyclopentyl-4,4-dimethyl-7-(4- |
| | morpholinopiperidin-1-yl)-3,4-dihydroisoquinoline- |
| | 2(1H)-carboxamide |
| 62 | 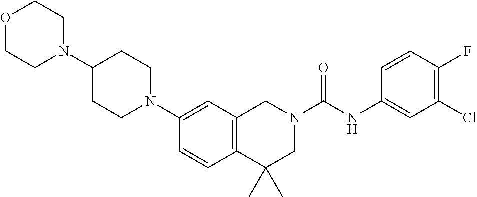 [SMILES: CC1(C)CN(C(=O)Nc2ccc(F)c(Cl)c2)Cc2cc(N3CCC(N4CCOCC4)CC3)ccc21] |
| | N-(3-chloro-4-fluorophenyl)-4,4-dimethyl-7-(4- |
| | morpholinopiperidin-1-yl)-3,4-dihydroisoquinoline- |
| | 2(1H)-carboxamide |
| 63 | 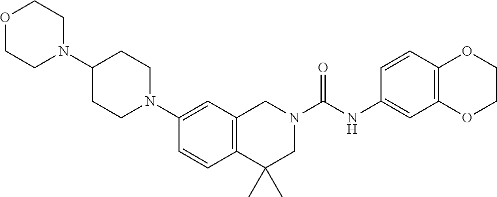 [SMILES: CC1(C)CN(C(=O)Nc2ccc3c(c2)OCCO3)Cc2cc(N3CCC(N4CCOCC4)CC3)ccc21] |
| | N-(2,3-dihydrobenzo[b][1,4]dioxin-6-yl)-4,4- |
| | dimethyl-7-(4-morpholinopiperidin-1-yl)-3,4- |
| | dihydroisoquinoline-2(1H)-carboxamide |
| 64 | 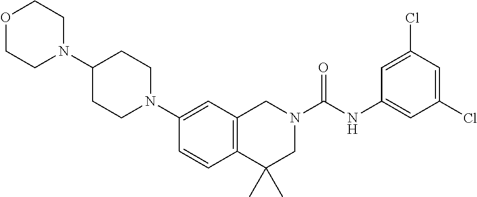 [SMILES: CC1(C)CN(C(=O)Nc2cc(Cl)cc(Cl)c2)Cc2cc(N3CCC(N4CCOCC4)CC3)ccc21] |
| | N-(3,5-dichlorophenyl)-4,4-dimethyl-7-(4- |
| | morpholinopiperidin-1-yl)-3,4-dihydroisoquinoline- |
| | 2(1H)-carboxamide |
| 65 | 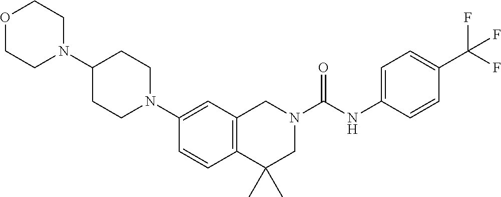 [SMILES: CC1(C)CN(C(=O)Nc2ccc(C(F)(F)F)cc2)Cc2cc(N3CCC(N4CCOCC4)CC3)ccc21] |
| | 4,4-dimethyl-7-(4-morpholinopiperidin-1-yl)-N-(4- |
| | (trifluoromethyl)phenyl)-3,4-dihydroisoquinoline- |
| | 2(1H)-carboxamide |
| 66 | 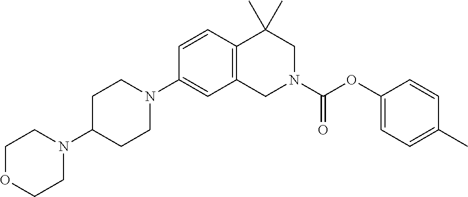 [SMILES: Cc1ccc(OC(=O)N2Cc3cc(N4CCC(N5CCOCC5)CC4)ccc3C(C)(C)C2)cc1] |
| | p-tolyl 4,4-dimethyl-7-(4-morpholinopiperidin-1- |
| | yl)-3,4-dihydroisoquinoline-2(1H)-carboxylate |
| 67 | 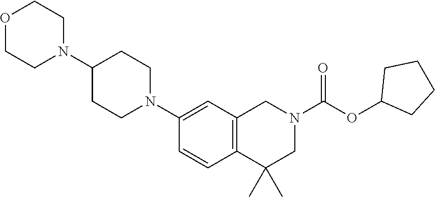 [SMILES: CC1(C)CN(C(=O)OC2CCCC2)Cc2cc(N3CCC(N4CCOCC4)CC3)ccc21] |
| | cyclopentyl 4,4-dimethyl-7-(4- |
| | morpholinopiperidin-1-yl)-3,4-dihydroisoquinoline- |
| | 2(1H)-carboxylate |
| 68 | 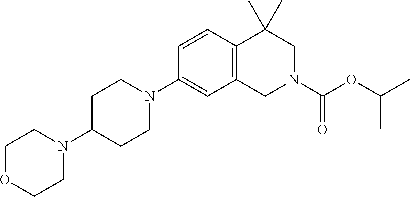 [SMILES: CC(C)OC(=O)N1Cc2cc(N3CCC(N4CCOCC4)CC3)ccc2C(C)(C)C1] |
| | isopropyl 4,4-dimethyl-7-(4-morpholinopiperidin- |
| | 1-yl)-3,4-dihydroisoquinoline-2(1H)-carboxylate |
| 69 | 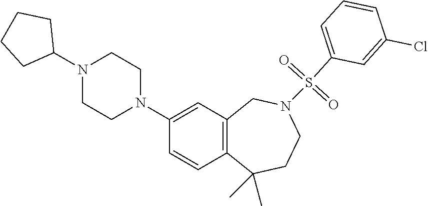 [SMILES: CC1(C)CCN(S(=O)(=O)c2cccc(Cl)c2)Cc2cc(N3CCN(C4CCCC4)CC3)ccc21] |
| | 2-((3-chlorophenyl)sulfonyl)-8-(4- |
| | cyclopentylpiperazin-1-yl)-5,5-dimethyl-2,3,4,5- |
| | tetrahydro-1H-benzo[c]azepine |
| 70 | 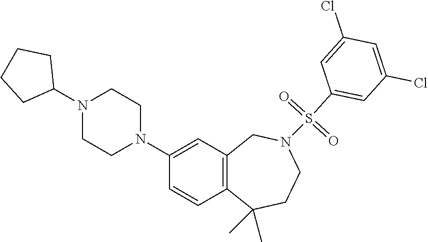 [SMILES: CC1(C)CCN(S(=O)(=O)c2cc(Cl)cc(Cl)c2)Cc2cc(N3CCN(C4CCCC4)CC3)ccc21] |
| | 8-(4-cyclopentylpiperazin-1-yl)-2-((3,5- |
| | dichlorophenyl)sulfonyl)-5,5-dimethyl-2,3,4,5- |
| | tetrahydro-1H-benzo[c]azepine |
| 71 | 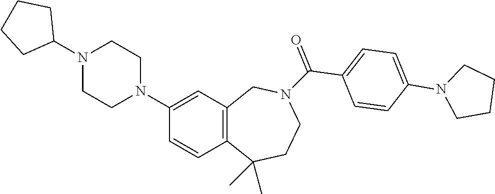 [SMILES: CC1(C)CCN(C(=O)c2ccc(N3CCCC3)cc2)Cc2cc(N3CCN(C4CCCC4)CC3)ccc21] |
| | (8-(4-cyclopentylpiperazin-1-yl)-5,5-dimethyl- |
| | 1,3,4,5-tetrahydro-2H-benzo[c]azepin-2-yl)(4- |
| | (pyrrolidin-1-yl)phenyl)methanone |
| 72 | 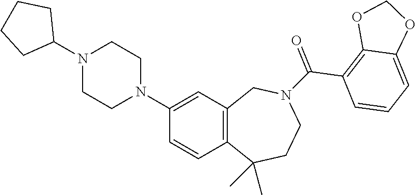 [SMILES: CC1(C)CCN(C(=O)c2cccc3c2OCO3)Cc2cc(N3CCN(C4CCCC4)CC3)ccc21] |
| | benzo[d][1,3]dioxol-4-yl(8-(4- |
| | cyclopentylpiperazin-1-yl)-5,5-dimethyl-1,3,4,5- |
| | tetrahydro-2H-benzo[c]azepin-2-yl)methanone |
| 73 | 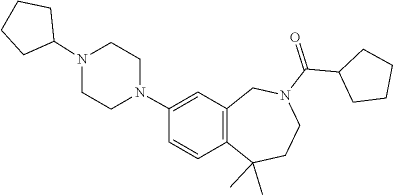 [SMILES: CC1(C)CCN(C(=O)C2CCCC2)Cc2cc(N3CCN(C4CCCC4)CC3)ccc21] |
| | cyclopentyl(8-(4-cyclopentylpiperazin-1-yl)-5,5- |
| | dimethyl-1,3,4,5-tetrahydro-2H-benzo[c]azepin-2- |
| | yl)methanone |
| 74 | 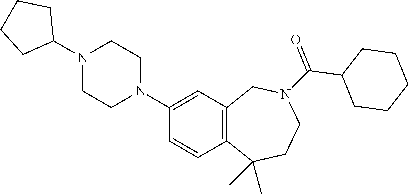 [SMILES: CC1(C)CCN(C(=O)C2CCCCC2)Cc2cc(N3CCN(C4CCCC4)CC3)ccc21] |
| | cyclohexyl(8-(4-cyclopentylpiperazin-1-yl)-5,5- |
| | dimethyl-1,3,4,5-tetrahydro-2H-benzo[c]azepin-2- |
| | yl)methanone |
| 75 | 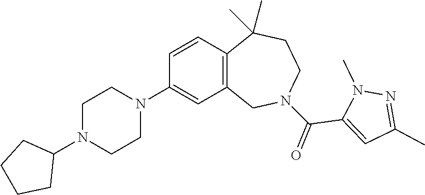 [SMILES: Cc1cc(C(=O)N2CCC(C)(C)c3ccc(N4CCN(C5CCCC5)CC4)cc3C2)n(C)n1] |
| | (8-(4-cyclopentylpiperazin-1-yl)-5,5-dimethyl- |
| | 1,3,4,5-tetrahydro-2H-benzo[c]azepin-2-yl)(1,3- |
| | dimethyl-1H-pyrazol-5-yl)methanone |
| 76 | 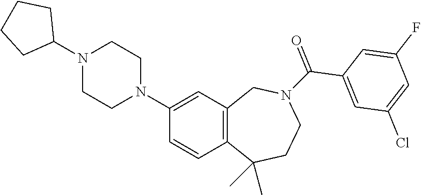 [SMILES: CC1(C)CCN(C(=O)c2cc(F)cc(Cl)c2)Cc2cc(N3CCN(C4CCCC4)CC3)ccc21] |
| | (3-chloro-5-fluorophenyl)(8-(4- |
| | cyclopentylpiperazin-1-yl)-5,5-dimethyl-1,3,4,5- |
| | tetrahydro-2H-benzo[c]azepin-2-yl)methanone |
| 77 | 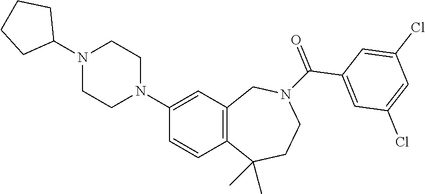 [SMILES: CC1(C)CCN(C(=O)c2cc(Cl)cc(Cl)c2)Cc2cc(N3CCN(C4CCCC4)CC3)ccc21] |
| | (8-(4-cyclopentylpiperazin-1-yl)-5,5-dimethyl- |
| | 1,3,4,5-tetrahydro-2H-benzo[c]azepin-2-yl)(3,5- |
| | dichlorophenyl)methanone |
| 78 | 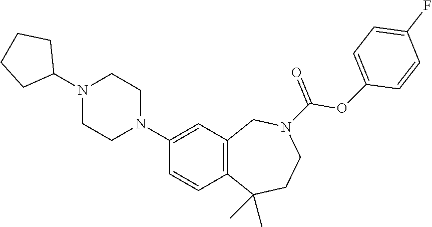 [SMILES: CC1(C)CCN(C(=O)Oc2ccc(F)cc2)Cc2cc(N3CCN(C4CCCC4)CC3)ccc21] |
| | 4-fluorophenyl 8-(4-cyclopentylpiperazin-1-yl)-5,5- |
| | dimethyl-1,3,4,5-tetrahydro-2H-benzo[c]azepine-2- |
| | carboxylate |
| 79 | 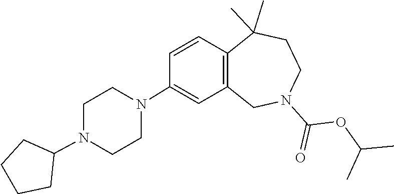 [SMILES: CC(C)OC(=O)N1CCC(C)(C)c2ccc(N3CCN(C4CCCC4)CC3)cc2C1] |
| | isopropyl 8-(4-cyclopentylpiperazin-1-yl)-5,5- |
| | dimethyl-1,3,4,5-tetrahydro-2H-benzo[c]azepine-2- |
| | carboxylate |
| 80 | 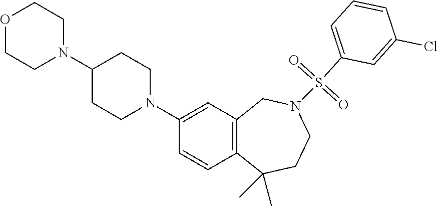 [SMILES: CC1(C)CCN(S(=O)(=O)c2cccc(Cl)c2)Cc2cc(N3CCC(N4CCOCC4)CC3)ccc21] |
| | 4-(1-(2-((3-chlorophenyl)sulfonyl)-5,5-dimethyl- |
| | 2,3,4,5-tetrahydro-1H-benzo[c]azepin-8- |
| | yl)piperidin-4-yl)morpholine |
| 81 | 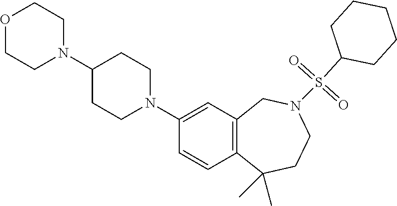 [SMILES: CC1(C)CCN(S(=O)(=O)C2CCCCC2)Cc2cc(N3CCC(N4CCOCC4)CC3)ccc21] |
| | 4-(1-(2-(cyclohexylsulfonyl)-5,5-dimethyl-2,3,4,5- |
| | tetrahydro-1H-benzo[c]azepin-8-yl)piperidin-4- |
| | yl)morpholine |
| 82 | 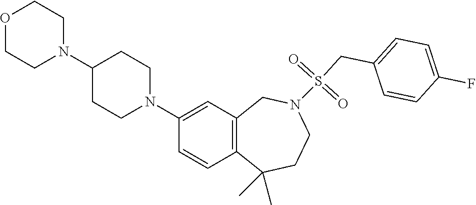 [SMILES: CC1(C)CCN(S(=O)(=O)Cc2ccc(F)cc2)Cc2cc(N3CCC(N4CCOCC4)CC3)ccc21] |
| | 4-(1-(2-((4-fluorobenzyl)sulfonyl)-5,5-dimethyl- |
| | 2,3,4,5-tetrahydro-1H-benzo[c]azepin-8- |
| | yl)piperidin-4-yl)morpholine |
| 83 | 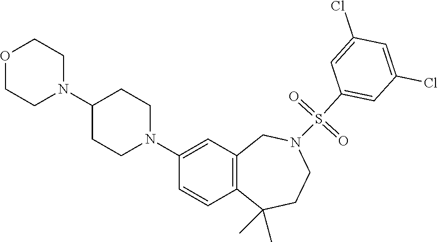 [SMILES: CC1(C)CCN(S(=O)(=O)c2cc(Cl)cc(Cl)c2)Cc2cc(N3CCC(N4CCOCC4)CC3)ccc21] |
| | 4-(1-(2-((3,5-dichlorophenyl)sulfonyl)-5,5- |
| | dimethyl-2,3,4,5-tetrahydro-1H-benzo[c]azepin-8- |
| | yl)piperidin-4-yl)morpholine |
| 84 | 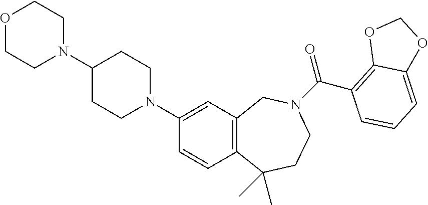 [SMILES: CC1(C)CCN(C(=O)c2cccc3c2OCO3)Cc2cc(N3CCC(N4CCOCC4)CC3)ccc21] |
| | benzo[d][1,3]dioxol-4-yl(5,5-dimethyl-8-(4- |
| | morpholinopiperidin-1-yl)-1,3,4,5-tetrahydro-2H- |
| | benzo[c]azepin-2-yl)methanone |
| 85 | 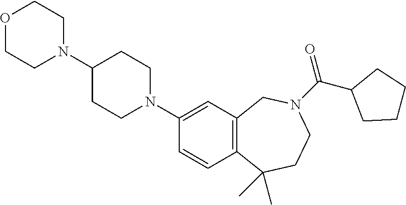 [SMILES: CC1(C)CCN(C(=O)C2CCCC2)Cc2cc(N3CCC(N4CCOCC4)CC3)ccc21] |
| | cyclopentyl(5,5-dimethyl-8-(4- |
| | morpholinopiperidin-1-yl)-1,3,4,5-tetrahydro-2H- |
| | benzo[c]azepin-2-yl)methanone |
| 86 | 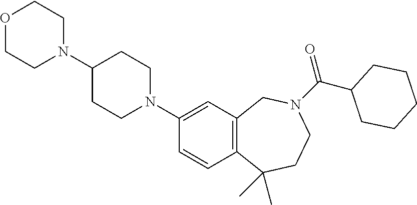 [SMILES: CC1(C)CCN(C(=O)C2CCCCC2)Cc2cc(N3CCC(N4CCOCC4)CC3)ccc21] |
| | cyclohexyl(5,5-dimethyl-8-(4-morpholinopiperidin- |
| | 1-yl)-1,3,4,5-tetrahydro-2H-benzo[c]azepin-2- |
| | yl)methanone |
| 87 | 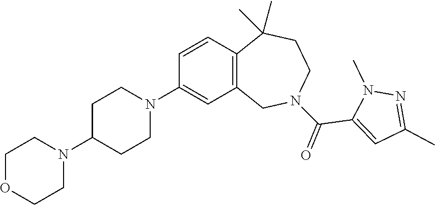 [SMILES: Cc1cc(C(=O)N2CCC(C)(C)c3ccc(N4CCC(N5CCOCC5)CC4)cc3C2)n(C)n1] |
| | (1,3-dimethyl-1H-pyrazol-5-yl)(5,5-dimethyl-8-(4- |
| | morpholinopiperidin-1-yl)-1,3,4,5-tetrahydro-2H- |
| | benzo[c]azepin-2-yl)methanone |
| 88 | 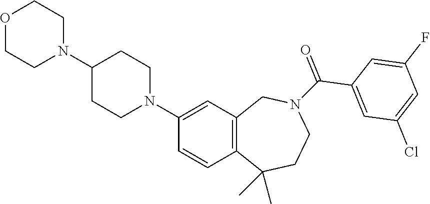 [SMILES: CC1(C)CCN(C(=O)c2cc(F)cc(Cl)c2)Cc2cc(N3CCC(N4CCOCC4)CC3)ccc21] |
| | (3-chloro-5-fluorophenyl)(5,5-dimethyl-8-(4- |
| | morpholinopiperidin-1-yl)-1,3,4,5-tetrahydro-2H- |
| | benzo[c]azepin-2-yl)methanone |
| 89 | 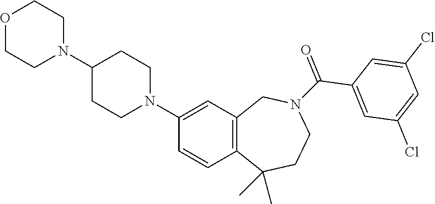 [SMILES: CC1(C)CCN(C(=O)c2cc(Cl)cc(Cl)c2)Cc2cc(N3CCC(N4CCOCC4)CC3)ccc21] |
| | (3,5-dichlorophenyl)(5,5-dimethyl-8-(4- |
| | morpholinopiperidin-1-yl)-1,3,4,5-tetrahydro-2H- |
| | benzo[c]azepin-2-yl)methanone |
| 90 | 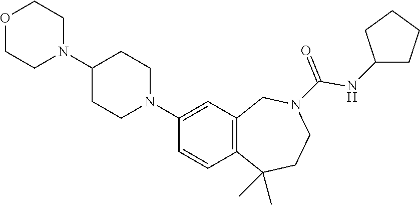 [SMILES: CC1(C)CCN(C(=O)NC2CCCC2)Cc2cc(N3CCC(N4CCOCC4)CC3)ccc21] |
| | N-cyclopentyl-5,5-dimethyl-8-(4- |
| | morpholinopiperidin-1-yl)-1,3,4,5-tetrahydro-2H- |
| | benzo[c]azepine-2-carboxamide |
| 91 | 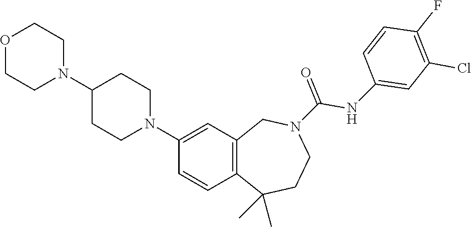 [SMILES: CC1(C)CCN(C(=O)Nc2ccc(F)c(Cl)c2)Cc2cc(N3CCC(N4CCOCC4)CC3)ccc21] |
| | N-(3-chloro-4-fluorophenyl)-5,5-dimethyl-8-(4- |
| | morpholinopiperidin-1-yl)-1,3,4,5-tetrahydro-2H- |
| | benzo[c]azepine-2-carboxamide |
| 92 | 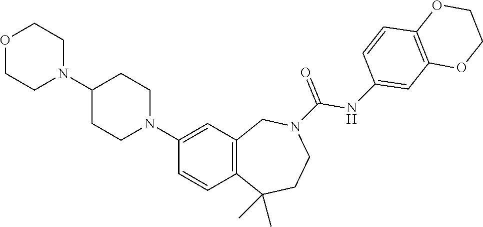 [SMILES: CC1(C)CCN(C(=O)Nc2ccc3c(c2)OCCO3)Cc2cc(N3CCC(N4CCOCC4)CC3)ccc21] |
| | N-(2,3-dihydrobenzo[b][1,4]dioxin-6-yl)-5,5- |
| | dimethyl-8-(4-morpholinopiperidin-1-yl)-1,3,4,5- |
| | tetrahydro-2H-benzo[c]azepine-2-carboxamide |
| 93 | 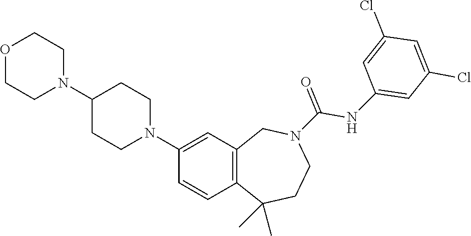 [SMILES: CC1(C)CCN(C(=O)Nc2cc(Cl)cc(Cl)c2)Cc2cc(N3CCC(N4CCOCC4)CC3)ccc21] |
| | N-(3,5-dichlorophenyl)-5,5-dimethyl-8-(4- |
| | morpholinopiperidin-1-yl)-1,3,4,5-tetrahydro-2H- |
| | benzo[c]azepine-2-carboxamide |
| 94 | 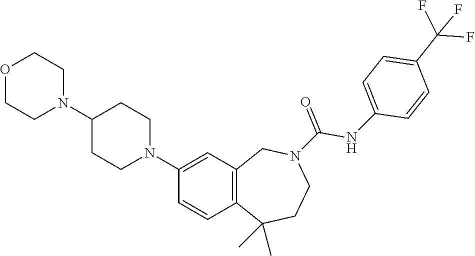 [SMILES: CC1(C)CCN(C(=O)Nc2ccc(C(F)(F)F)cc2)Cc2cc(N3CCC(N4CCOCC4)CC3)ccc21] |
| | 5,5-dimethyl-8-(4-morpholinopiperidin-1-yl)-N-(4- |
| | (trifluoromethyl)phenyl)-1,3,4,5-tetrahydro-2H- |
| | benzo[c]azepine-2-carboxamide |
| 95 | 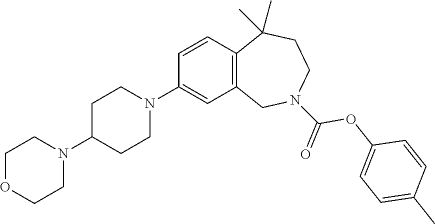 [SMILES: Cc1ccc(OC(=O)N2CCC(C)(C)c3ccc(N4CCC(N5CCOCC5)CC4)cc3C2)cc1] |
| | p-tolyl 5,5-dimethyl-8-(4-morpholinopiperidin-1- |
| | yl)-1,3,4,5-tetrahydro-2H-benzo[c]azepine-2- |
| | carboxylate |
| 96 | 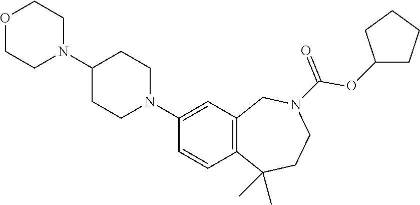 [SMILES: CC1(C)CCN(C(=O)OC2CCCC2)Cc2cc(N3CCC(N4CCOCC4)CC3)ccc21] |
| | cyclopentyl 5,5-dimethyl-8-(4- |
| | morpholinopiperidin-1-yl)-1,3,4,5-tetrahydro-2H- |
| | benzo[c]azepine-2-carboxylate |
| 97 | 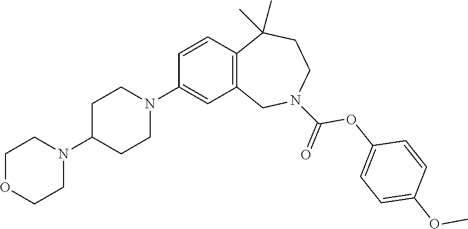 [SMILES: COc1ccc(OC(=O)N2CCC(C)(C)c3ccc(N4CCC(N5CCOCC5)CC4)cc3C2)cc1] |
| | 4-methoxyphenyl 5,5-dimethyl-8-(4- |
| | morpholinopiperidin-1-yl)-1,3,4,5-tetrahydro-2H- |
| | benzo[c]azepine-2-carboxylate |
| 98 | 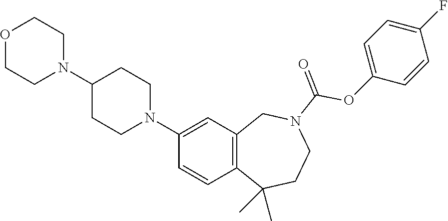 [SMILES: CC1(C)CCN(C(=O)Oc2ccc(F)cc2)Cc2cc(N3CCC(N4CCOCC4)CC3)ccc21] |
| | 4-fluorophenyl 5,5-dimethyl-8-(4- |
| | morpholinopiperidin-1-yl)-1,3,4,5-tetrahydro-2H- |
| | benzo[c]azepine-2-carboxylate |
| 99 | 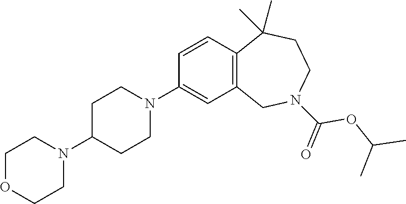 [SMILES: CC(C)OC(=O)N1CCC(C)(C)c2ccc(N3CCC(N4CCOCC4)CC3)cc2C1] |
| | isopropyl 5,5-dimethyl-8-(4-morpholinopiperidin- |
| | 1-yl)-1,3,4,5-tetrahydro-2H-benzo[c]azepine-2- |
| | carboxylate |
| 100 | 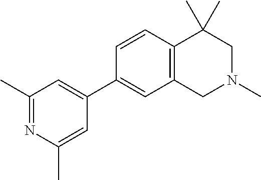 |
| | 7-(2,6-dimethylpyridin-4-yl)-2,4,4-trimethyl- |
| | 1,2,3,4-tetrahydroisoquinoline |
| 101 | 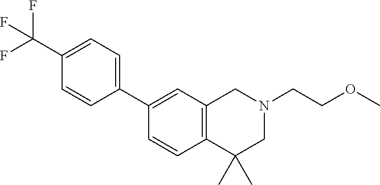 [SMILES: COCCN1Cc2cc(-c3ccc(C(F)(F)F)cc3)ccc2C(C)(C)C1] |
| | 2-(2-methoxyethyl)-4,4-dimethyl-7-(4- |
| | (trifluoromethyl)phenyl)-1,2,3,4- |
| | tetrahydroisoquinoline |
| 102 | 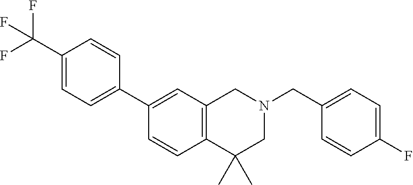 [SMILES: CC1(C)CN(Cc2ccc(F)cc2)Cc2cc(-c3ccc(C(F)(F)F)cc3)ccc21] |
| | 2-(4-fluorobenzyl)-4,4-dimethyl-7-(4- |
| | (trifluoromethyl)phenyl)-1,2,3,4- |
| | tetrahydroisoquinoline |
| 103 | 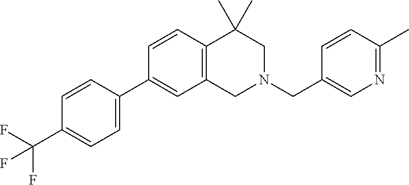 [SMILES: Cc1ccc(CN2Cc3cc(-c4ccc(C(F)(F)F)cc4)ccc3C(C)(C)C2)cn1] |
| | 4,4-dimethyl-2-((6-methylpyridin-3-yl)methyl)-7- |
| | (4-(trifluoromethyl)phenyl)-1,2,3,4- |
| | tetrahydroisoquinoline |
| 104 | 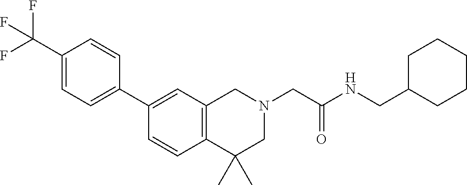 [SMILES: CC1(C)CN(CC(=O)NCC2CCCCC2)Cc2cc(-c3ccc(C(F)(F)F)cc3)ccc21] |
| | N-(cyclohexylmethyl)-2-(4,4-dimethyl-7-(4- |
| | (trifluoromethyl)phenyl)-3,4-dihydroisoquinolin- |
| | 2(1H)-yl)acetamide |
| 105 | 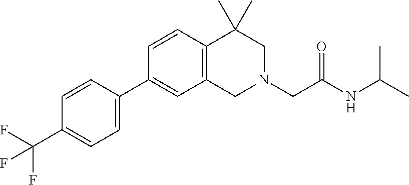 [SMILES: CC(C)NC(=O)CN1Cc2cc(-c3ccc(C(F)(F)F)cc3)ccc2C(C)(C)C1] |
| | 2-(4,4-dimethyl-7-(4-(trifluoromethyl)phenyl)-3,4- |
| | dihydroisoquinolin-2(1H)-yl)-N- |
| | isopropylacetamide |
| 106 | 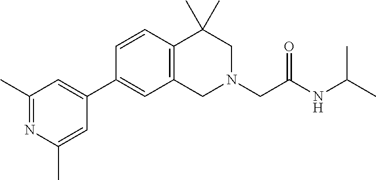 [SMILES: Cc1cc(-c2ccc3c(c2)CN(CC(=O)NC(C)C)CC3(C)C)cc(C)n1] |
| | 2-(7-(2,6-dimethylpyridin-4-yl)-4,4-dimethyl-3,4- |
| | dihydroisoquinolin-2(1H)-yl)-N- |
| | isopropylacetamide |
| 107 | 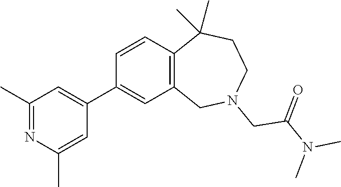 |
| | 2-(8-(2,6-dimethylpyridin-4-yl)-5,5-dimethyl- |
| | 1,3,4,5-tetrahydro-2H-benzo[c]azepin-2-yl)-N,N- |
| | dimethylacetamide |
| 108 | 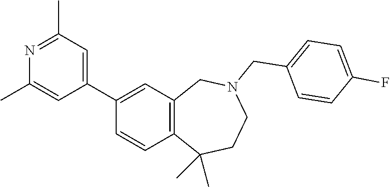 [SMILES: Cc1cc(-c2ccc3c(c2)CN(Cc2ccc(F)cc2)CCC3(C)C)cc(C)n1] |
| | 8-(2,6-dimethylpyridin-4-yl)-2-(4-fluorobenzyl)- |
| | 5,5-dimethyl-2,3,4,5-tetrahydro-1H- |
| | benzo[c]azepine |
| 109 | 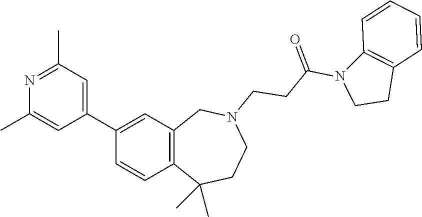 [SMILES: Cc1cc(-c2ccc3c(c2)CN(CCC(=O)N2CCc4ccccc42)CCC3(C)C)cc(C)n1] |
| | 3-(8-(2,6-dimethylpyridin-4-yl)-5,5-dimethyl- |
| | 1,3,4,5-tetrahydro-2H-benzo[c]azepin-2-yl)-1- |
| | (indolin-1-yl)propan-1-one |
| 110 | 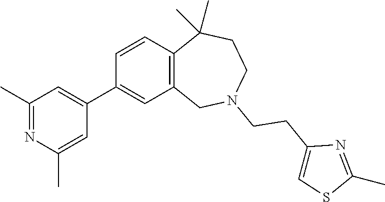 |
| | 4-(2-(8-(2,6-dimethylpyridin-4-yl)-5,5-dimethyl- |
| | 1,3,4,5-tetrahydro-2H-benzo[c]azepin-2-yl)ethyl)- |
| | 2-methylthiazole |
| 111 | 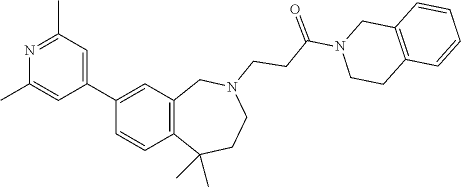 [SMILES: Cc1cc(-c2ccc3c(c2)CN(CCC(=O)N2CCc4ccccc4C2)CCC3(C)C)cc(C)n1] |
| | 1-(3,4-dihydroisoquinolin-2(1H)-yl)-3-(8-(2,6- |
| | dimethylpyridin-4-yl)-5,5-dimethyl-1,3,4,5- |
| | tetrahydro-2H-benzo[c]azepin-2-yl)propan-1-one |
| 112 | 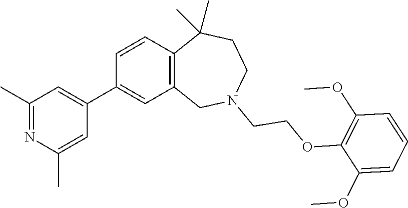 [SMILES: COc1cccc(OC)c1OCCN1CCC(C)(C)c2ccc(-c3cc(C)nc(C)c3)cc2C1] |
| | 2-(2-(2,6-dimethoxyphenoxy)ethyl)-8-(2,6- |
| | dimethylpyridin-4-yl)-5,5-dimethyl-2,3,4,5- |
| | tetrahydro-1H-benzo[c]azepine |
| 113 | 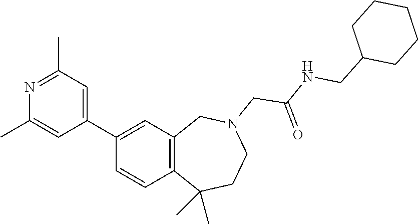 [SMILES: Cc1cc(-c2ccc3c(c2)CN(CC(=O)NCC2CCCCC2)CCC3(C)C)cc(C)n1] |
| | N-(cyclohexylmethyl)-2-(8-(2,6-dimethylpyridin-4- |
| | yl)-5,5-dimethyl-1,3,4,5-tetrahydro-2H- |
| | benzo[c]azepin-2-yl)acetamide |
| 114 | 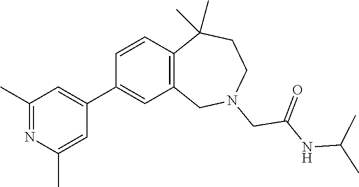 [SMILES: Cc1cc(-c2ccc3c(c2)CN(CC(=O)NC(C)C)CCC3(C)C)cc(C)n1] |
| | 2-(8-(2,6-dimethylpyridin-4-yl)-5,5-dimethyl- |
| | 1,3,4,5-tetrahydro-2H-benzo[c]azepin-2-yl)-N- |
| | isopropylacetamide |
| 115 | 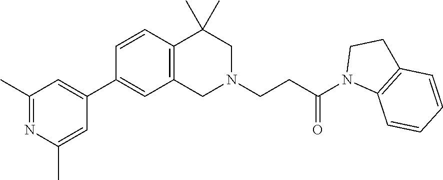 [SMILES: Cc1cc(-c2ccc3c(c2)CN(CCC(=O)N2CCc4ccccc42)CC3(C)C)cc(C)n1] |
| | 3-(7-(2,6-dimethylpyridin-4-yl)-4,4-dimethyl-3,4- |
| | dihydroisoquinolin-2(1H)-yl)-1-(indolin-1- |
| | yl)propan-1-one |
| 116 | 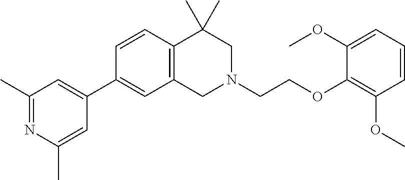 |
| | 2-(2-(2,6-dimethoxyphenoxy)ethyl)-7-(2,6- |
| | dimethylpyridin-4-yl)-4,4-dimethyl-1,2,3,4- |
| | tetrahydroisoquinoline |
| 117 | 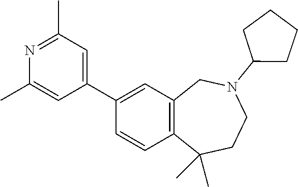 [SMILES: Cc1cc(-c2ccc3c(c2)CN(C2CCCC2)CCC3(C)C)cc(C)n1] |
| | 2-cyclopentyl-8-(2,6-dimethylpyridin-4-yl)-5,5- |
| | dimethyl-2,3,4,5-tetrahydro-1H-benzo[c]azepine |
| 118 | 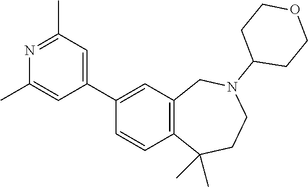 [SMILES: Cc1cc(-c2ccc3c(c2)CN(C2CCOCC2)CCC3(C)C)cc(C)n1] |
| | 8-(2,6-dimethylpyridin-4-yl)-5,5-dimethyl-2- |
| | (tetrahydro-2H-pyran-4-yl)-2,3,4,5-tetrahydro-1H- |
| | benzo[c]azepine |
| 119 | 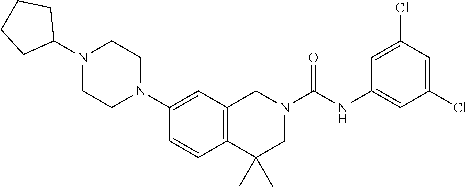 [SMILES: CC1(C)CN(C(=O)Nc2cc(Cl)cc(Cl)c2)Cc2cc(N3CCN(C4CCCC4)CC3)ccc21] |
| | 7-(4-cyclopentylpiperazin-1-yl)-N-(3,5- |
| | dichlorophenyl)-4,4-dimethyl-3,4- |
| | dihydroisoquinoline-2(1H)-carboxamide |
| 120 | 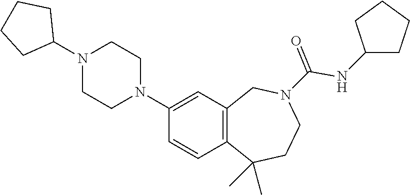 [SMILES: CC1(C)CCN(C(=O)NC2CCCC2)Cc2cc(N3CCN(C4CCCC4)CC3)ccc21] |
| | N-cyclopentyl-8-(4-cyclopentylpiperazin-1-yl)-5,5- |
| | dimethyl-1,3,4,5-tetrahydro-2H-benzo[c]azepine-2- |
| | carboxamide |
| 121 | 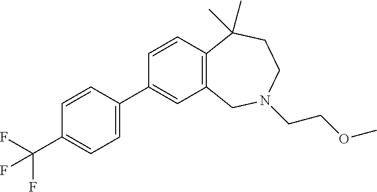 [SMILES: COCCN1CCC(C)(C)c2ccc(-c3ccc(C(F)(F)F)cc3)cc2C1] |
| | 2-(2-methoxyethyl)-5,5-dimethyl-8-(4- |
| | (trifluoromethyl)phenyl)-2,3,4,5-tetrahydro-1H- |
| | benzo[c]azepine |
| 122 | 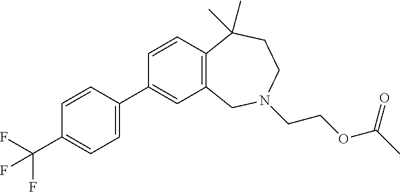 [SMILES: CC(=O)OCCN1CCC(C)(C)c2ccc(-c3ccc(C(F)(F)F)cc3)cc2C1] |
| | 2-(5,5-dimethyl-8-(4-(trifluoromethyl)phenyl)- |
| | 1,3,4,5-tetrahydro-2H-benzo[c]azepin-2-yl)ethyl |
| | acetate |
| 123 | 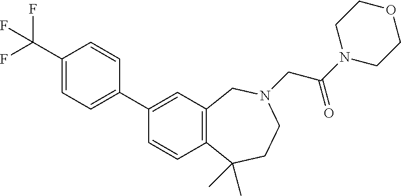 [SMILES: CC1(C)CCN(CC(=O)N2CCOCC2)Cc2cc(-c3ccc(C(F)(F)F)cc3)ccc21] |
| | 2-(5,5-dimethyl-8-(4-(trifluoromethyl)phenyl)- |
| | 1,3,4,5-tetrahydro-2H-benzo[c]azepin-2-yl)-1- |
| | morpholinoethan-1-one |
| 124 | 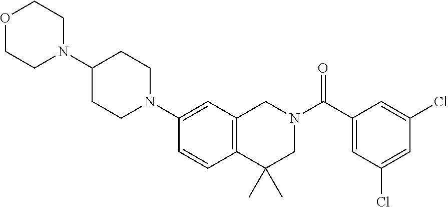 [SMILES: CC1(C)CN(C(=O)c2cc(Cl)cc(Cl)c2)Cc2cc(N3CCC(N4CCOCC4)CC3)ccc21] |
| | (3,5-dichlorophenyl)(4,4-dimethyl-7-(4- |
| | morpholinopiperidin-1-yl)-3,4-dihydroisoquinolin- |
| | 2(1H)-yl)methanone |
| 125 | 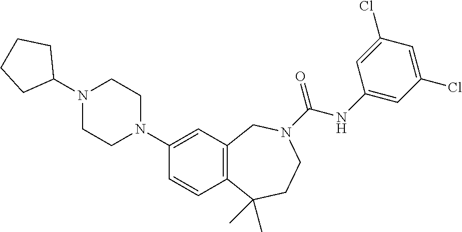 [SMILES: CC1(C)CCN(C(=O)Nc2cc(Cl)cc(Cl)c2)Cc2cc(N3CCN(C4CCCC4)CC3)ccc21] |
| | 8-(4-cyclopentylpiperazin-1-yl)-N-(3,5- |
| | dichlorophenyl)-5,5-dimethyl-1,3,4,5-tetrahydro- |
| | 2H-benzo[c]azepine-2-carboxamide |
| 126 | 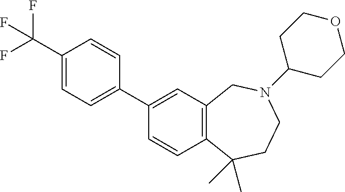 [SMILES: CC1(C)CCN(C2CCOCC2)Cc2cc(-c3ccc(C(F)(F)F)cc3)ccc21] |
| | 5,5-dimethyl-2-(tetrahydro-2H-pyran-4-yl)-8-(4- |
| | (trifluoromethyl)phenyl)-2,3,4,5-tetrahydro-1H- |
| | benzo[c]azepine |
| 127 | 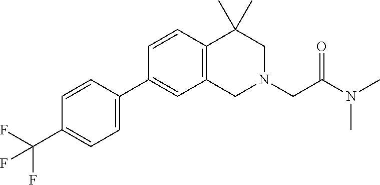 [SMILES: CN(C)C(=O)CN1Cc2cc(-c3ccc(C(F)(F)F)cc3)ccc2C(C)(C)C1] |
| | 2-(4,4-dimethyl-7-(4-(trifluoromethyl)phenyl)-3,4- |
| | dihydroisoquinolin-2(1H)-yl)-N,N- |
| | dimethylacetamide |
| 128 | 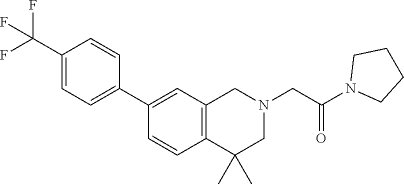 [SMILES: CC1(C)CN(CC(=O)N2CCCC2)Cc2cc(-c3ccc(C(F)(F)F)cc3)ccc21] |
| | 2-(4,4-dimethyl-7-(4-(trifluoromethyl)phenyl)-3,4- |
| | dihydroisoquinolin-2(1H)-yl)-1-(pyrrolidin-1- |
| | yl)ethan-1-one |
| 129 | 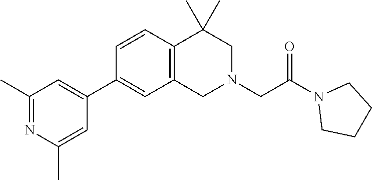 [SMILES: Cc1cc(-c2ccc3c(c2)CN(CC(=O)N2CCCC2)CC3(C)C)cc(C)n1] |
| | 2-(7-(2,6-dimethylpyridin-4-yl)-4,4-dimethyl-3,4- |
| | dihydroisoquinolin-2(1H)-yl)-1-(pyrrolidin-1- |
| | yl)ethan-1-one |
| 130 | 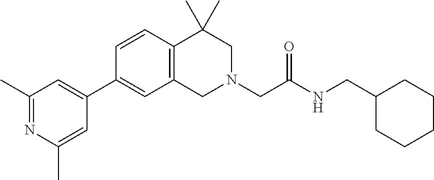 [SMILES: Cc1cc(-c2ccc3c(c2)CN(CC(=O)NCC2CCCCC2)CC3(C)C)cc(C)n1] |
| | N-(cyclohexylmethyl)-2-(7-(2,6-dimethylpyridin-4- |
| | yl)-4,4-dimethyl-3,4-dihydroisoquinolin-2(1H)- |
| | yl)acetamide |
| 131 | 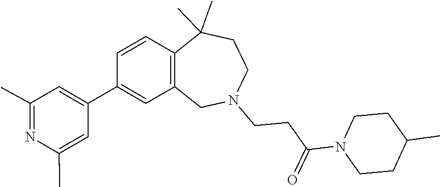 |
| | 3-(8-(2,6-dimethylpyridin-4-yl)-5,5-dimethyl- |
| | 1,3,4,5-tetrahydro-2H-benzo[c]azepin-2-yl)-1-(4- |
| | methylpiperidin-1-yl)propan-1-one |
| 132 | 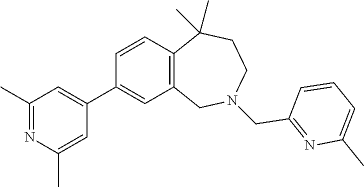 |
| | 8-(2,6-dimethylpyridin-4-yl)-5,5-dimethyl-2-((6- |
| | methylpyridin-2-yl)methyl)-2,3,4,5-tetrahydro-1H- |
| | benzo[c]azepine |
| 133 | 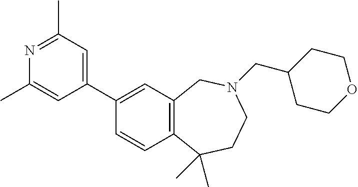 [SMILES: Cc1cc(-c2ccc3c(c2)CN(CC2CCOCC2)CCC3(C)C)cc(C)n1] |
| | 8-(2,6-dimethylpyridin-4-yl)-5,5-dimethyl-2- |
| | ((tetrahydro-2H-pyran-4-yl)methyl)-2,3,4,5- |
| | tetrahydro-1H-benzo[c]azepine |
| 134 | 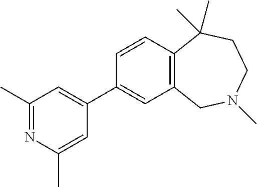 |
| | 8-(2,6-dimethylpyridin-4-yl)-2,5,5-trimethyl- |
| | 2,3,4,5-tetrahydro-1H-benzo[c]azepine |
| 135 | 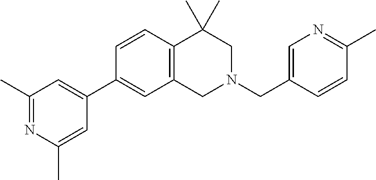 |
| | 7-(2,6-dimethylpyridin-4-yl)-4,4-dimethyl-2-((6- |
| | methylpyridin-3-yl)methyl)-1,2,3,4- |
| | tetrahydroisoquinoline |
| 136 | 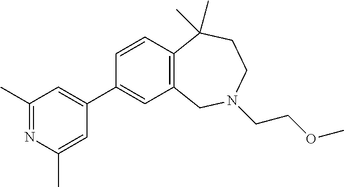 |
| | 8-(2,6-dimethylpyridin-4-yl)-2-(2-methoxyethyl)- |
| | 5,5-dimethyl-2,3,4,5-tetrahydro-1H- |
| | benzo[c]azepine |
| 137 | 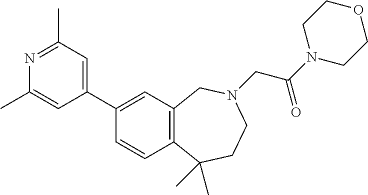 [SMILES: Cc1cc(-c2ccc3c(c2)CN(CC(=O)N2CCOCC2)CCC3(C)C)cc(C)n1] |
| | 2-(8-(2,6-dimethylpyridin-4-yl)-5,5-dimethyl- |
| | 1,3,4,5-tetrahydro-2H-benzo[c]azepin-2-yl)-1- |
| | morpholinoethan-1-one |

In some embodiments, the compound of Formula (I) is Compound No. 10, i.e.:

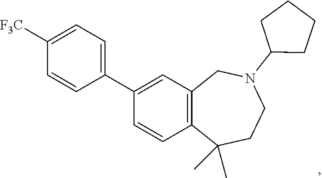 [SMILES: CC1(C)CCN(C2CCCC2)Cc2cc(-c3ccc(C(F)(F)F)cc3)ccc21]

2-cyclopentyl-5,5-dimethyl-8-(4-(trifluoromethyl)phenyl)-2,3,4,5-tetrahydro-1H-benzo[c]azepine, and referred to herein as “CB”.

The preparation of the compounds of the disclosure can be found in PCT Application Publication No. WO 2022/036080, and US Patent Application Publication No. 2023/0054070, both of which are incorporated by reference in their entireties for all purposes.

In some embodiments, the preparation of a compound of the disclosure is described below in Scheme I and Scheme II:

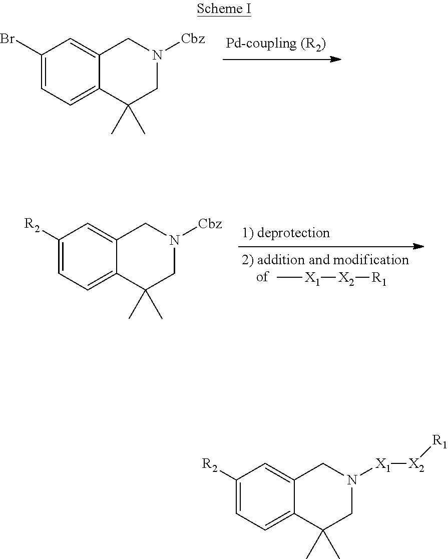

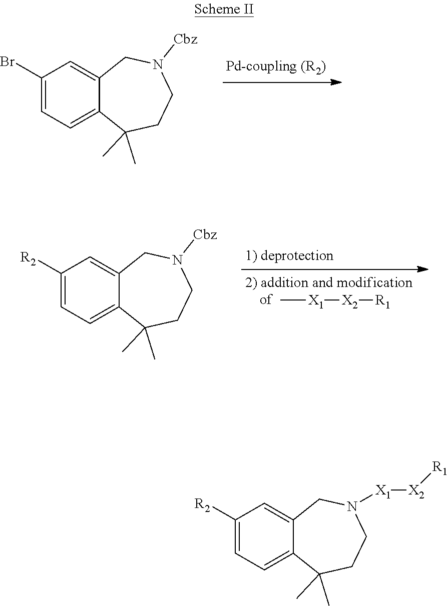

### Methods of the Present Disclosure

The disclosure provides methods of treating lysosomal storage disorders, or a symptom of a lysosomal storage disorder (LSD), in a subject in need thereof, wherein the method comprises administering to the subject a therapeutically effective amount of Formula (I), MIN-101-B, or a pharmaceutically acceptable salt, prodrug, or solvate thereof.

The disclosure also provides methods of preventing a lysosomal storage disorder, or a symptom of lysosomal storage disorder (LSD), in a subject in need thereof, wherein the method comprises administering to the subject a prophylactically effective amount of Formula (I), MIN-101-B, or a pharmaceutically acceptable salt, prodrug, or solvate thereof.

The present disclosure also provides methods of treating an illness resulting from a lysosomal storage disorder, comprising administering to an individual in need thereof a therapeutically effective amount of a compound of Formula (I), MIN-101-B, or a pharmaceutically acceptable salt, prodrug, or solvate thereof.

The present disclosure also provides methods of preventing an illness resulting from a lysosomal storage disorder, comprising administering to an individual in need thereof a therapeutically effective amount of a compound of Formula (I), MIN-101-B, or a pharmaceutically acceptable salt, prodrug, or solvate thereof.

In some embodiments, the present disclosure provides a compound of Formula (I), MIN-101-B, or a pharmaceutically acceptable salt, prodrug, or solvate thereof, for use in treating or preventing a lysosomal storage disorder in a subject, a symptom of a lysosomal storage disorder, or an illness resulting from a lysosomal storage disorder.

In some embodiments, the present disclosure provides a compound of Formula (I), MIN-101-B, or a pharmaceutically acceptable salt, prodrug, or solvate thereof, for use in treating or preventing a disease or disorder with symptoms similar to those of a lysosomal storage disorder.

In some embodiments, the present disclosure provides the use of a compound of Formula (I), MIN-101-B, or a pharmaceutically acceptable salt, prodrug, or solvate thereof, in the manufacture of a medicament, for the treatment or prevention of a lysosomal storage disorder in a subject, a symptom of a lysosomal storage disorder, or an illness resulting from a lysosomal storage disorder.

The disclosure provides methods of treating lysosomal storage disorders, or a symptom of a lysosomal storage disorder (LSD), in a subject in need thereof, wherein the method comprises administering to the subject a therapeutically effective amount of Formula (I) or a pharmaceutically acceptable salt thereof.

The disclosure also provides methods of preventing a lysosomal storage disorder, or a symptom of lysosomal storage disorder (LSD), in a subject in need thereof, wherein the method comprises administering to the subject a prophylactically effective amount of Formula (I) or a pharmaceutically acceptable salt thereof.

The present disclosure also provides methods of treating an illness resulting from a lysosomal storage disorder, comprising administering to an individual in need thereof a therapeutically effective amount of a compound of Formula (I) or a pharmaceutically acceptable salt thereof.

The present disclosure also provides methods of preventing an illness resulting from a lysosomal storage disorder, comprising administering to an individual in need thereof a therapeutically effective amount of a compound of Formula (I) or a pharmaceutically acceptable salt thereof.

In some embodiments, the present disclosure provides a compound of Formula (I), or a pharmaceutically acceptable salt thereof, for use in treating or preventing a lysosomal storage disorder in a subject, a symptom of a lysosomal storage disorder, or an illness resulting from a lysosomal storage disorder.

In some embodiments, the present disclosure provides a compound of Formula (I), or a pharmaceutically acceptable salt thereof, for use in treating or preventing a disease or disorder with symptoms similar to those of a lysosomal storage disorder.

In some embodiments, the present disclosure provides the use of a compound of Formula (I) or a pharmaceutically acceptable salt thereof, in the manufacture of a medicament, for the treatment or prevention of a lysosomal storage disorder in a subject, a symptom of a lysosomal storage disorder, or an illness resulting from a lysosomal storage disorder.

The disclosure provides methods of treating lysosomal storage disorders, or a symptom of a lysosomal storage disorder (LSD), in a subject in need thereof, wherein the method comprises administering to the subject a therapeutically effective amount of Formula (I).

The disclosure also provides methods of preventing a lysosomal storage disorder, or a symptom of lysosomal storage disorder (LSD), in a subject in need thereof, wherein the method comprises administering to the subject a prophylactically effective amount of Formula (I).

The present disclosure also provides methods of treating an illness resulting from a lysosomal storage disorder, comprising administering to an individual in need thereof a therapeutically effective amount of a compound of Formula (I).

The present disclosure also provides methods of preventing an illness resulting from a lysosomal storage disorder, comprising administering to an individual in need thereof a therapeutically effective amount of a compound of Formula (I).

In some embodiments, the present disclosure provides a compound of Formula (I) for use in treating or preventing a lysosomal storage disorder in a subject, a symptom of a lysosomal storage disorder, or an illness resulting from a lysosomal storage disorder.

In some embodiments, the present disclosure provides a compound of Formula (I) for use in treating or preventing a disease or disorder with symptoms similar to those of a lysosomal storage disorder.

In some embodiments, the present disclosure provides the use of a compound of Formula (I) in the manufacture of a medicament, for the treatment or prevention of a lysosomal storage disorder in a subject, a symptom of a lysosomal storage disorder, or an illness resulting from a lysosomal storage disorder.

In some embodiments, the lysosomal storage disorder is cholesteryl ester storage disease, Wolman disease, Hunter syndrome, Hurler's disease, Hurler syndrome, Scheie syndrome, Hurler-Scheie syndrome, mucopolysaccharidosis (MPS) type III (SanFilippo syndrome), Morquio syndrome A, Morquio syndrome B, Maroteaux-Lamy syndrome, Sly syndrome, Fabry disease, Gaucher disease, Krabbe disease (globoid cell leukodystrophy), metachromatic leukodystrophy, Niemann-Pick disease, Sandhoff disease, Tay-Sachs disease, Alpha mannosidosis, Beta mannosidosis, Schindler disease, aspartylglucosaminuria, fucosidosis, neuronal ceroid lipofuscinosis (NCL), galactosialidosis (enzyme protection protein defect), infantile sialic acid storage disease, Salla disease (transmembrane protein defect), sialuria, mucolipidosis, cystinosis, Danon disease, Multiple sulfatase deficiency, proteins GM2-AP deficiency (variant AB), Farber disease, lysosomal acid lipase deficiency (early onset or late onset), Morquio (MPS IVA/Type A), Morquio (MPS IVB/Type B), hyaluronidase deficiency, Pycnodysostosis, or Pompe disease.

In some embodiments, the MPS type III is MPS type IIIA (caused by a deficiency of sulfamidase), MPS type IIIB (caused by a deficiency of α-N-acetylglucosaminidase), MPS type IIIC (caused by a deficiency of heparan acetyl COA: α-glucosaminide N-acetyltransferase), MPS type IIID (caused by a deficiency of N-acetylglucosamine 6-sulfatase); or MPS type IIIE (caused by a deficiency of N-glucosamine 3-O-sulfatase) (see Fedele, A. O. “Sanfilippo syndrome: causes, consequences, and treatments,” Appl Clin Genet. 2015 Nov. 25; 8:269-81. doi: 10.2147/TACG.S57672. PMID: 26648750; PMCID: PMC4664539.).

In some embodiments, the Gaucher disease is Gaucher disease type I, Gaucher disease type II, or Gaucher disease type III.

In some embodiments, the neuronal ceroid lipofuscinosis (NCL) is Type 1 NCL (i.e., infantile NCL or Santavuori-Haltia disease), Type 2 NCL (i.e., late infantile NCL or Jansky-Bielschowsky disease), Type 3 NCL (i.e., juvenile NCL or Batten disease), Type 4 NCL (i.e., adult NCL or Kufs disease), Type 5 NCL (i.e., Finnish late infantile NCL), Type 6 NCL (i.e., variant late infantile NCL), Type 7 NCL, Type 8 NCL (i.e., Northern epilepsy or progressive epilepsy with mental retardation), Type 8 NCL Turkish late infantile variant, Type 9 NCL, Type 10 NCL, Type 11 NCL, Type 12 NCL, Type 13 NCL, or Type 14 NCL.

In some embodiments, the mucolipidosis is sialidosis type I (i.e., cherry-red spot myoclonus syndrome), sialidosis type II (i.e., mucolipidosis type I), I-cell disease (i.e., mucolipidosis type II), pseudo-Hurler-polydystrophy (i.e., mucolipidosis type III), or mucolipidosis type IV.

In some embodiments, the Niemann-Pick disease is Niemann-Pick disease type A, Niemann-Pick disease type B, or Niemann-Pick disease type C. In some embodiments, the Niemann-Pick disease type C is Niemann-Pick disease type C1. In some embodiments, the Niemann-Pick disease type C is Niemann-Pick disease type C2.

In some embodiments, the LSD is characterized by a mutation in the gene encoding NPC intracellular cholesterol transporter 1 (NPC1). NPC1 is a gene encoding a protein that resides in the limiting membrane of endosomes and lysosome and mediates intracellular cholesterol trafficking though binding of cholesterol to its N-terminal domain. In some embodiments the mutation in NPC1 results in Niemann-Pick disease type C. In some embodiments, the NPC1 mutation is a T₃₁₈₂→C transition in exon 21. In some embodiments, this mutation leads to a 11061T substitution. In some embodiments an I1061T substitution affects a putative transmembrane domain protein in NPC1. In some embodiments the I1061T substitution leads to hepatosplenomegaly and severe progressive neurological dysfunction in the subject. (See Millat, G. et. al. “Niemann-Pick C₁ disease: the 11061T substitution is a frequent mutant allele in patients of Western European descent and correlates with a classic juvenile phenotype” Am J Hum Genet. 1999 November; 65 (5): 1321-9. doi: 10.1086/302626. PMID: 10521297; PMCID: PMC1288284.)

In some embodiments, the administration of a therapeutically effective amount of the compound of Formula (I), MIN-101-B, or a pharmaceutically acceptable salt, prodrug, or solvate thereof does not result in a change in NPC1 expression in the subject.

In some embodiments, the LSD is characterized by a mutation in the gene encoding the gene encoding NPC intracellular cholesterol transporter 2 (NPC2) gene, the gene encoding sphingomyelin phosphodiesterase 1 (SMPD1) or the gene encoding acid sphingomyelinase (ASM).

In some embodiments, the ASM mutation is a H421Y mutation, a K576N mutation, a L137P mutation, a fsP189 mutation, a L549P mutation, a S379P mutation, a R441X mutation, a R474W mutation, a F480L mutation, a A196P mutation, a DeltaR608 mutation, a R496L mutation, a fsP330 mutation, or a L302P mutation (https://nnpdf.org/diseases/).

In some embodiments, symptoms of a lysosomal storage disorder comprise dyslipidemia (in some embodiments the dyslipidemia is high cholesterol or low cholesterol), hepatosplenomegaly (i.e., an enlarged liver and spleen), ataxia, vertical supranuclear gaze palsy, hypotonia, liver disease, respiratory infection, difficulty with speech, difficulty with swallowing and feeding, loss of cognitive skills, seizure, or any combination thereof.

In some embodiments, the LSD results in an increase in cholesterol levels, cholesterol content, and/or cholesterol accumulation in a subject when compared to a healthy subject. In some embodiments, the increase in cholesterol is associated with an increase in low density lipoprotein (LDL) particles. In some embodiments the increase in cholesterol results in an increase in lysosomal size as measured by geometric mean ratio fold change when compared to healthy lysosomes.

In some embodiments, the LSD results in a decrease in cholesterol levels, cholesterol content, and/or cholesterol accumulation in a subject when compared to a healthy subject. In some embodiments, the decrease in cholesterol is associated with a decrease in low density lipoprotein (LDL) particles.

In some embodiments the cholesterol levels are determined using Filipin staining. In some embodiments, the cholesterol levels are determined through analysis of a patient blood sample in a lipid panel.

In some embodiments, the subject is an adult (i.e., 18 years of age or older).

In some embodiments, the subject is a pediatric subject (i.e., less than 18 years of age).

In some embodiments the subject has a lysosomal storage disorder. In some embodiments, the subject does not have a lysosomal storage disorder, but has one or more diseases with symptoms similar to those of a lysosomal storage disorder. In some embodiments, the symptoms include high cholesterol. In some embodiments, the symptoms include low cholesterol. In some embodiments, the subject has cancer, chronic kidney disease, Parkinson's disease, diabetes, HIV/AIDS, lupus, abetalipoproteinemia, or Alzheimer's disease.

In some embodiments, provided herein are methods of modulating cholesterol in a subject in need thereof, wherein the method comprises administering to the subject a therapeutically effective amount of the compound of Formula (I), MIN-101-B, or a pharmaceutically acceptable salt, prodrug, or solvate thereof. In some embodiments, provided herein are methods of modulating cholesterol levels in a subject in need thereof, wherein the method comprises administering to the subject a therapeutically effective amount of the compound of Formula (I), MIN-101-B, or a pharmaceutically acceptable salt, prodrug, or solvate thereof. In some embodiments, provided herein are methods of modulating cholesterol content in a subject in need thereof, wherein the method comprises administering to the subject a therapeutically effective amount of the compound of Formula (I), MIN-101-B, or a pharmaceutically acceptable salt, prodrug, or solvate thereof. In some embodiments, provided herein are methods of modulating cholesterol accumulation in a subject in need thereof, wherein the method comprises administering to the subject a therapeutically effective amount of the compound of Formula (I), MIN-101-B, or a pharmaceutically acceptable salt, prodrug, or solvate thereof.

In some embodiments, the modulation is an increase in cholesterol, cholesterol levels, cholesterol content, or cholesterol accumulation in the subject. In some embodiments, the modulation is a decrease in in cholesterol, cholesterol levels, cholesterol content, or cholesterol accumulation in the subject.

In some embodiments, provided herein are methods of modulating lysosomal size in a subject in need thereof, wherein the method comprises administering to the subject a therapeutically effective amount of the compound of Formula (I), MIN-101-B, or a pharmaceutically acceptable salt, prodrug, or solvate thereof.

In some embodiments, the modulation in lysosomal size is an increase in lysosomal size in the subject. In some embodiments, the modulation in lysosomal size is a decrease in lysosomal size in the subject.

In some embodiments, provided herein are methods of simultaneously modulating cholesterol accumulation and lysosomal size in a subject in need thereof, wherein the method comprises administering to the subject a therapeutically effective amount of the compound of Formula (I), MIN-101-B, or a pharmaceutically acceptable salt, prodrug, or solvate thereof.

In some embodiments, the modulation is an increase in both lysosomal size and cholesterol accumulation in the subject. In some embodiments, the modulation is a decrease in both lysosomal size and cholesterol accumulation in the subject. In some embodiments the modulation is an increase in lysosomal size and a decrease in cholesterol accumulation in the subject. In some embodiments, the modulation is a decrease in lysosomal size and an increase in cholesterol accumulation in the subject.

In some embodiments, the administration of the compound of Formula (I), MIN-101-B, or a pharmaceutically acceptable salt, prodrug, or solvate thereof to the subject results in: (i) a reduction of cholesterol accumulation in the subject, and/or (ii) a reduction of the lysosomal size of the subject.

In some embodiments, the administration of the compound of Formula (I), MIN-101-B, or a pharmaceutically acceptable salt, prodrug, or solvate thereof to the subject results in: (i) an increase of cholesterol accumulation in the subject, and/or (ii) an increase of the lysosomal size of the subject.

In some embodiments, the administration of the compound of Formula (I), MIN-101-B, or a pharmaceutically acceptable salt, prodrug, or solvate thereof to the subject results in a reduction of cholesterol accumulation in the subject. In some embodiments, the administration of the compound of Formula (I), MIN-101-B, or a pharmaceutically acceptable salt, prodrug, or solvate thereof to the subject results in a reduction of the lysosomal size of the subject. In some embodiments, the administration of the compound of Formula (I), MIN-101-B, or a pharmaceutically acceptable salt, prodrug, or solvate thereof to the subject results in a reduction of the lysosomal size and the cholesterol accumulation of the subject. In some embodiments, the administration of the compound of Formula (I), MIN-101-B, or a pharmaceutically acceptable salt, prodrug, or solvate thereof to the subject results in a reduction of the lysosomal size in the brain, spleen, liver, lungs, and bone marrow of the subject.

In some embodiments, the administration of the compound of Formula (I), MIN-101-B, or a pharmaceutically acceptable salt, prodrug, or solvate thereof to the subject results in an increase of cholesterol accumulation in the subject. In some embodiments, the administration of the compound of Formula (I), MIN-101-B, or a pharmaceutically acceptable salt, prodrug, or solvate thereof to the subject results in an increase of the lysosomal size of the subject. In some embodiments, the administration of the compound of Formula (I), MIN-101-B, or a pharmaceutically acceptable salt, prodrug, or solvate thereof to the subject results in an increase of the lysosomal size and the cholesterol accumulation of the subject. In some embodiments, the administration of the compound of Formula (I), MIN-101-B, or a pharmaceutically acceptable salt, prodrug, or solvate thereof to the subject results in an increase of the lysosomal size in the brain, spleen, liver, lungs, and bone marrow of the subject.

In some embodiments, the cholesterol accumulation in the subject is modulated by at least 10%, at least 20%, at least 30%, at least 40%, at least 50%, at least 60%, or at least 70% after administration of the compound of Formula (I), MIN-101-B, or a pharmaceutically acceptable salt, prodrug, or solvate thereof, to the subject.

In some embodiments, the cholesterol accumulation is modulated in the subject by about 10%, about 20%, about 30%, about 40%, about 50%, about 60%, or about 70% after administration of the compound of Formula (I), MIN-101-B, or a pharmaceutically acceptable salt, prodrug, or solvate thereof, to the subject.

In some embodiments, the cholesterol accumulation is modulated in the subject by 10-80%, 20-70%, 30-60%, or 40-50% after administration of the compound of Formula (I), MIN-101-B, or a pharmaceutically acceptable salt, prodrug, or solvate thereof, to the subject.

In some embodiments, the lysosomal size is modulated by at least 10%, at least 20%, at least 30%, at least 40%, at least 50%, at least 60%, or at least 70% after administration of the compound of Formula (I), MIN-101-B, or a pharmaceutically acceptable salt, prodrug, or solvate thereof, to the subject.

In some embodiments, the lysosomal size is modulated about 10%, about 20%, about 30%, about 40%, about 50%, about 60%, or about 70% after administration of the compound of Formula (I), MIN-101-B, or a pharmaceutically acceptable salt, prodrug, or solvate thereof, to the subject.

In some embodiments, the lysosomal size is determined using fluorescence microscopy. In some embodiments, the lysosomal size is determined through cytofluorimetric assays using Lysotracker™ (Life Technology #L7528) fluorescence as a tool. LysoTracker™ provides fluorescence detection for live-cell staining of acidic environments and can be used for labeling and tracing acidic organelles such as lysosomes. In some embodiments, stained and unstained cells can be analyzed by Cytofluorimeter CytoFLEX (Beckman Coulter). In some embodiments, the data provided by the Cytofluorimeter CytoFLEX analysis is the ratio of geometric (geo) mean of LysoTracker™ levels in stained divided by unstained samples. In some embodiments the lysosomal size is determined using electron microscopy.

In some embodiments, the modulation of cholesterol accumulation in the subject occurs within 24 hours, within 48 hours, or within 72 hours of the administration of the compound of Formula (I), MIN-101-B, or a pharmaceutically acceptable salt, prodrug, or solvate thereof.

In some embodiments, the modulation of lysosomal size occurs to the subject within 24 hours, within 48 hours, or within 72 hours of the administration of the compound of Formula (I), MIN-101-B, or a pharmaceutically acceptable salt, prodrug, or solvate thereof.

The disclosure provides methods of treating Alzheimer's disease, or a symptom of Alzheimer's disease, in a subject in need thereof, wherein the method comprises administering to the subject a therapeutically effective amount of the compound of Formula (I), MIN-101-B, or a pharmaceutically acceptable salt, prodrug, or solvate thereof.

The disclosure also provides methods of preventing Alzheimer's disease, or a symptom of Alzheimer's disease, in a subject in need thereof, wherein the method comprises administering to the subject a prophylactically effective amount of the compound of Formula (I), MIN-101-B, or a pharmaceutically acceptable salt, prodrug, or solvate thereof.

In some embodiments, the therapeutically effective amount of the compound of Formula (I), MIN-101-B, or a pharmaceutically acceptable salt, prodrug, or solvate thereof, is between about 1 mg to about 1000 mg, between about 20 mg to about 900 mg, between about 50 mg to about 800 mg, between about 150 mg to about 700 mg, between about 200 mg to about 650 mg, between about 300 mg to about 600 mg, or between about 400 mg to about 500 mg.

In some embodiments, the therapeutically effective amount of the compound of Formula (I), MIN-101-B, or a pharmaceutically acceptable salt, prodrug, or solvate thereof, is between 1 mg to 1000 mg, between 20 mg to 900 mg, between 50 mg to 800 mg, between 150 mg to 700 mg, between 200 mg to 650 mg, between 300 mg to 600 mg, or between 400 mg to 500 mg.

In some embodiments, the therapeutically effective amount of the compound of Formula (I), MIN-101-B, or a pharmaceutically acceptable salt, prodrug, or solvate thereof, is about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1,000 mg.

In some embodiments, the therapeutically effective amount of the compound of Formula (I), MIN-101-B, or a pharmaceutically acceptable salt, prodrug, or solvate thereof, is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1,000 mg.

In some embodiments, the therapeutically effective amount of the compound of Formula (I), MIN-101-B, or a pharmaceutically acceptable salt, prodrug, or solvate thereof, is administered to the subject once, twice, three times, or more a day.

In some embodiments, the therapeutically effective amount of the compound of Formula (I), MIN-101-B, or a pharmaceutically acceptable salt, prodrug, or solvate thereof is administered to the subject once or twice a day.

In some embodiments, the therapeutically effective amount of the compound of Formula (I), MIN-101-B, or a pharmaceutically acceptable salt, prodrug, or solvate thereof is administered to the subject once a day.

In some embodiments, the therapeutically effective amount of the compound of Formula (I), MIN-101-B, or a pharmaceutically acceptable salt, prodrug, or solvate thereof is administered to the subject twice a day.

In some embodiments, the therapeutically effective amount of the compound of Formula (I), MIN-101-B, or a pharmaceutically acceptable salt, prodrug, or solvate thereof is administered orally to the subject.

In some embodiments the subject is administered the compound of Formula (I), MIN-101-B, or a pharmaceutically acceptable salt, prodrug, or solvate thereof, in combination with one or more LSD treatments. In some embodiments, the one or more LSD treatments comprises treatment with recombinant enzymes or enzymes alone or in combination with chaperones, fusion proteins such as HIRMAb, miglustat, gene therapy, intravenous administration of insulin growth factor II fusion protein, stem cell transplants, enzyme replacement therapy, substrate reduction therapy, pharmacological chaperone therapy, immunosuppressants, nonsteroidal anti-inflammatory drugs (NSAIDs), or any combination thereof. See Beck, M. (2018), Treatment strategies for lysosomal storage disorders. Dev Med Child Neurol, 60:13-18.

Compounds designed, selected and/or optimized by methods described above, once produced, can be characterized using a variety of assays known to those skilled in the art to determine whether the compounds have biological activity. For example, the molecules can be characterized by conventional assays, including, but not limited to, those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

### EXAMPLES

### Example 1

### Compounds of Formula (I)

Compounds of Formula (I) are synthesized as described in PCT Application Publication No. WO 2022/036080, and US Patent Application Publication No. 2023/0054070, both of which are incorporated by reference in their entireties for all purposes.

### Example 2

### Cell Culture

Healthy and I1061T fibroblasts (Coriell Institute Camden USA, cod. GM05659 (Healthy) and GM18453 (11061T)) were grown in air containing 5% CO₂ in Dulbecco's Modified Eagle Medium (DMEM) medium, containing 10% v/v fetal calf serum, 2 mM L-glutamine, 0.1 mg/mL gentamicin, and 100 U/mL penicillin. Cells were passaged every 3 days.

### Effect of Compound No. 10 on NPC1 Protein Levels

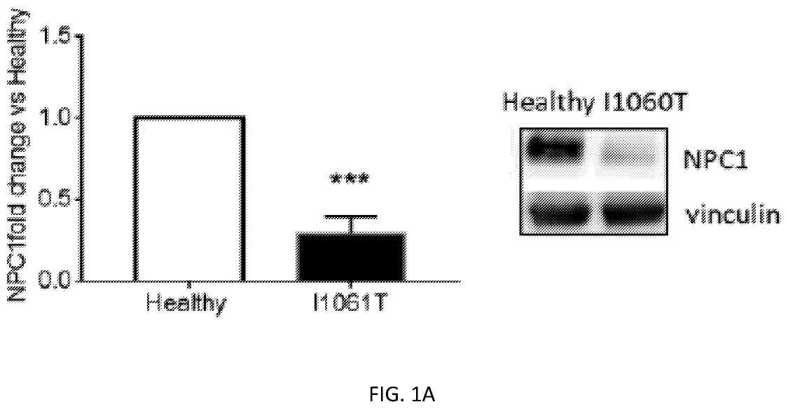

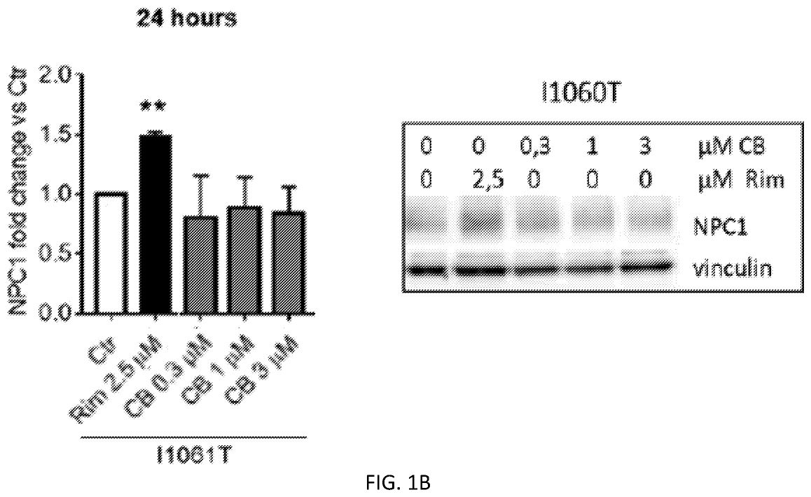

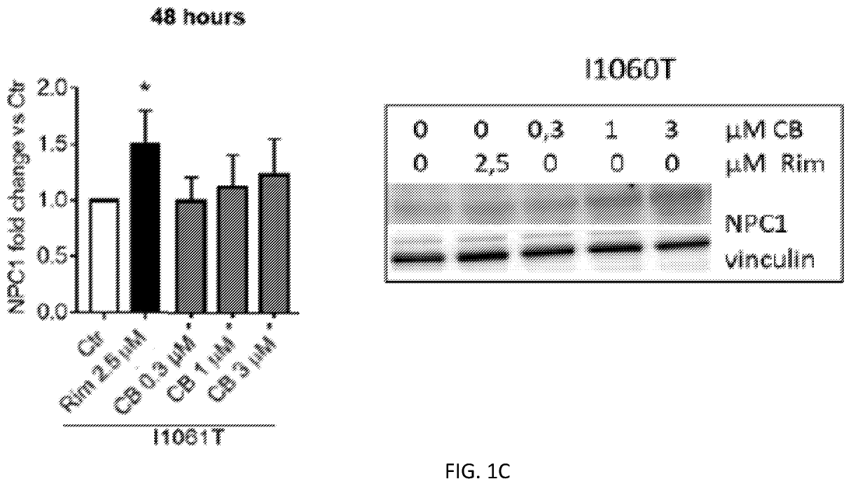

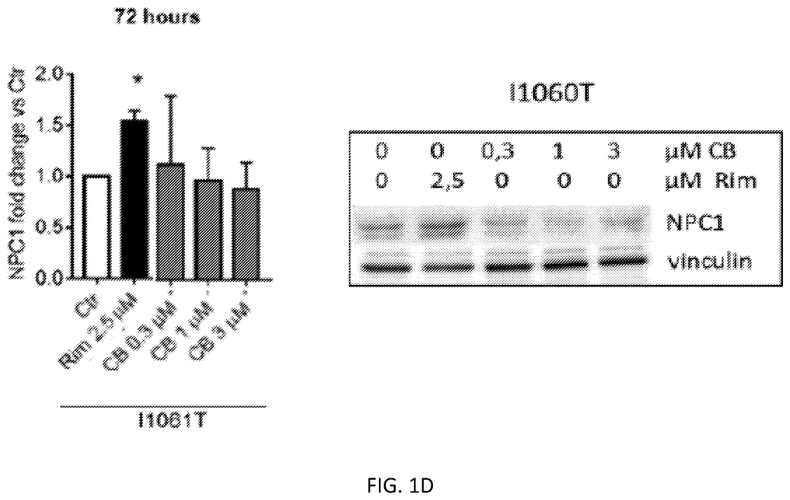

To evaluate the effect Compound No. 10 (“CB”) on NPC1 protein levels, I1061T cells were treated for 24 hours (FIG. 1B), 48 hours (FIG. 1C), and 72 hours (FIG. 1D) with CB at three different concentrations (0.3 μM, 1 μM, and 3 μM), a vehicle control (Ctr), or a positive control, 2.5 μM Rimcazole (Rim) (Tocris #1497). 1.5 mg of each of the compounds were weighed, dissolved in the proper solvent to reach a 3 mM concentration, kept at 4° C., and used within 15 days. The dilutions for the treatment were carried out in order to administer a 1:1000 volume. Specifically, 2 μL in 2 mL medium was administered. Ctr was made with DMSO.

### Western Blot Analysis

Cells were washed twice in phosphate buffer saline (PBS), harvested with 1% v/v trypsin, and pelleted by centrifugation. Pellets were re-suspended in PBS containing 0.1 M sucrose, 0.05 M KCl, 0.04 M KH₂PO₄, 30 mM EDTA, pH 7.4, and sonicated. The protein concentration was determined by the Lowry method.

Samples were solubilized in 0.25 mM Tris-HCl, (pH 6.8) containing 20% (w/v) SDS, protease inhibitor cocktail (Merck #PIC0002), then boiled for 2 minutes. 10 μg of solubilized proteins were resolved by SDS-PAGE at 100 V for 1 hour and then electrophoretically transferred to nitrocellulose with Turboblot (Biorad) for 10 min at 1.3 A and 25 V. The nitrocellulose was treated with 3% (w/v) bovine serum albumin (BSA) in Tris Buffered Saline (20 mM Trizma base, 137 mM NaCl, 0.1%, v/v, Tween-20, pH 7.6) and then probed over night with the anti-NPC1 antibodies (1:5000 Novus Biological #400148). The nitrocellulose was successively incubated for 1 h at room temperature with HRP-conjugated secondary anti-rabbit antibodies (Bio-Rad Laboratories, Milan, Italy). Protein-bound antibodies were visualized by clarity Enhanced Chemiluminescence (ECL) Western blotting (Bio-Rad Laboratories, Milan, Italy, #1705061), and chemiluminescence was registered through the ChemiDoc MP system (Bio-Rad Laboratories, Milan, Italy). Densitometric analysis derived from Western blots was then carried out using ImageJ version 1.52t (National Institutes of Health, Bethesda, MD, USA) software for Windows. Vinculin was used as housekeeping proteins, which served as internal controls for protein loading. Densitometric calculations were obtained as arbitrary units, derived from the ratio between the intensity of protein band and the housekeeping protein.

Without wishing to be bound by theory, FIG. 1A demonstrates that there was a strong difference between healthy and I1061T fibroblasts in terms of NPC1 protein content demonstrating that the above method is sensitive in detecting it. FIGS. 1B-1D demonstrate that Compound No. 10 did not have a statistically significant effect on NPC1 protein expression within the timeframe and conditions described in Example 1 indicating that physiological changes which occur after treatment with Compound No. 10 may be independent of the function of NPC1.

### Example 3

### LysoTracker™ Analysis

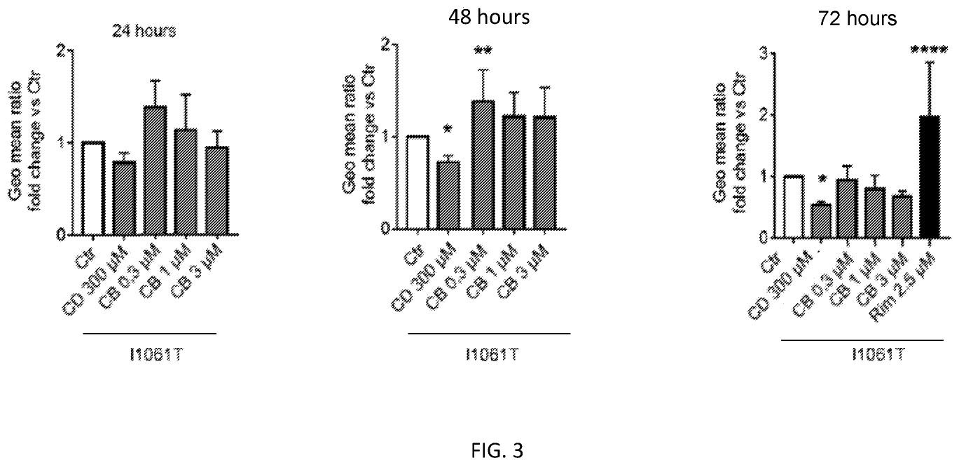

Healthy and I1061T fibroblasts were cultured as in Example 1. To evaluate the effect of Compound No. 10 on lysosomal size, cells were treated with either 300 μM beta-cyclodextrin (CD), 2.5 μM Rim, 0.3 μM Compound No. 10 (CB), 1 μM Compound No. 10, 3 μM Compound No. 10, or vehicle control (ctr) for 72 hours. Following treatment, cytofluorimetric assays using Lysotracker™ (Life Technology #L7528) fluorescence as a tool was used and performed according to the manufacturer's instructions. LysoTracker™ provides fluorescence detection for live-cell staining of acidic environments and can be used for labeling and tracing acidic organelles such as lysosomes. Stained and unstained cells were analyzed by Cytofluorimeter CytoFLEX (Beckman Coulter). Data provided are the ratio of geometric (geo) mean of LysoTracker™ levels in stained divided by unstained samples, collected at 24 hours (FIG. 3, left), 48 hours (FIG. 3, middle), and 72 hours (FIG. 3, right).

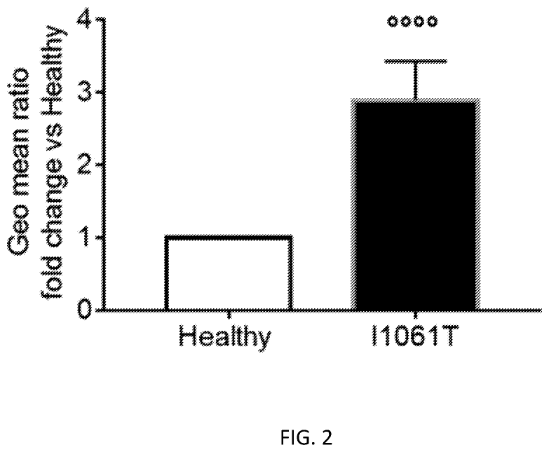

Without wishing to be bound by theory, Example 2 demonstrates that there was a statistically significant increase in the lysosomal size of I1061T fibroblasts when compared to healthy cells (FIG. 2). Treatment with Compound No. 10 showed an increase in fluorescence of the lysosomes in the 11061T fibroblasts after 48 hours, particularly at a concentration of 0.3 μM, representing an increase in lysosomal size (FIG. 3).

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A method of treating or preventing a lysosomal storage disorder, or a symptom of a lysosomal storage disorder, in a subject in need thereof, wherein the method comprises administering to the subject a therapeutically effective amount of a compound of Formula (I): 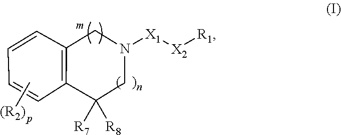
or a pharmaceutically acceptable salt thereof, wherein
X₁ is absent, —(CR₃R₄)_{q}—, —O—, —S(O)₂—, —C(O)—, —C(S)—, —S(O)—, —NR₃C(O)—, —C(O) NR₃—, —C(O)O—, or —OC(O)—;
X₂ is absent, —(CR₃R₄)_{q}—, —O—, —S(O₂)—, —C(O)—, —C(S)—, —S(O)—, —NR₃C(O)—, —C(O) NR₃—, —C(O)O—, or —OC(O)—;
R₁ is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, 5- to 12-membered heteroaryl comprising 1-6 heteroatoms selected from N, O, and S, 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₅;
each R₂ independently is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆;
each R₃ independently is H, halogen, or C₁-C₆ alkyl;
each R₄ independently is H, halogen, or C₁-C₆ alkyl;
each R₅ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
each R₆ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with one or more —C(O) C₁-C₆ alkyl, —C(O)OC₁-C₆ alkyl, —C(O)NH₂, —C(O)NHC₁-C₆ alkyl, or —C(O)N(C₁-C₆alkyl)₂;
R₇ is C₁-C₆ alkyl optionally substituted with one or more OH, halogen, or C₁-C₆ alkoxyl;
R₈ is C₁-C₆ alkyl optionally substituted with one or more OH, halogen, or C₁-C₆ alkoxyl; or
R₇ and R₈, together with the carbon atom they are attached, form a C₃-C₆ cycloalkyl optionally substituted with one or more OH, halogen, or C₁-C₆ alkoxyl;
m is 0, 1, 2, or 3;
n is 0, 1, 2, or 3;
p is 0, 1, 2, 3, or 4; and
each q independently is 1, 2, 3, or 4.

2. The method of claim 1, wherein the compound is a compound of Formula (Ia): 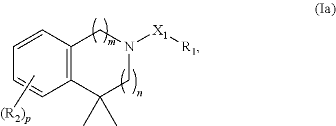
or a pharmaceutically acceptable salt thereof, wherein
X₁ is absent, (CR₃R₄)_{q}, —C(O)—, —C(S)—, —S(O)—, —NR₃C(O)—, or —C(O)O—;
R₁ is H, OH, halogen, NH₂, C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is optionally substituted with one or more R₅;
each R₂ independently is H, OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆;
each R₃ independently is H or halogen;
each R₄ independently is H or halogen;
each R₅ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
each R₆ independently is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, C₆-C₁₀ aryl, or 5- to 14-membered saturated or unsaturated heterocycloalkyl comprising 1-5 heteroatoms selected from N, O, and S; wherein the alkyl, alkenyl, alkynyl, cycloalkyl, or aryl of any of the foregoing is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂;
m is 0, 1, 2, or 3;
n is 0, 1, 2, or 3;
p is 0, 1, 2, 3, or 4; and
q is 1, 2, 3, or 4.

3. The method of claim 1 or 2, wherein X₁ is (CR₃R₄)_{q}, —C(O)—, —C(S)—, —S(O)—, —NR₃C(O)—, or —C(O)O—.

4. The method of claim 1 or 2, wherein X₁ is absent.

5. The method of claim 1 or 2, wherein X₁ is absent, (CR₃R₄)_{q}, —C(O)—, or —C(O)O—.

6. The method of claim 1 or 2, wherein X₁ is (CR₃R₄)_{q}.

7. The method of claim 1 or 2, wherein X₁ is —C(O)— or —C(O)O—.

8. The method of claim 1 or 2, wherein X₁ is —C(O)—.

9. The method of claim 1 or 2, wherein X₁ is —C(O)O—.

10. The method of claim 1 or 2, wherein X₁ is —NR₃C(O)— or —C(O) NR₃—.

11. The method of claim 1 or 2, wherein X₁ is —O— or —S(O)₂—.

12. The method of claim 1, wherein X₂ is (CR₃R₄)_{q}, —C(O)—, —C(S)—, —S(O)—, —NR₃C(O)—, or —C(O)O—.

13. The method of claim 1, wherein X₂ is absent.

14. The method of claim 1, wherein X₂ is absent, (CR₃R₄)_{q}, —C(O)—, or —C(O)O—.

15. The method of claim 1, wherein X₂ is (CR₃R₄)_{q}.

16. The method of claim 1, wherein X₂ is —C(O)— or —C(O)O—.

17. The method of claim 1, wherein X₂ is —C(O)—.

18. The method of claim 1, wherein X₂ is —C(O)O—.

19. The method of claim 1, wherein X₂ is —NR₃C(O)— or —C(O) NR₃—.

20. The method of claim 1, wherein X₂ is —O— or —S(O)₂—.

21. The method of any one of claims 1-20, wherein R₁ is H or OH.

22. The method of any one of claims 1-20, wherein R₁ is halogen.

23. The method of any one of claims 1-20, wherein R₁ is C₁-C₆ alkyl, C₂-C₆ alkenyl, C₂-C₆ alkynyl, C₁-C₆ haloalkyl, C₁-C₆ alkoxyl, C₁-C₆ haloalkoxyl, or C₃-C₆ cycloalkyl, wherein the alkyl, alkenyl, alkynyl, haloalkyl, alkoxyl, haloalkoxyl, or cycloalkyl of any of the foregoing is optionally substituted with one or more R₅.

24. The method of any one of claims 1-20, wherein R₁ is C₁-C₆ alkyl, C₂-C₆ alkenyl, or C₂-C₆ alkynyl, wherein the alkyl, alkenyl, or alkynyl of any of the foregoing is optionally substituted with one or more R₅.

25. The method of any one of claims 1-20, wherein R₁ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is optionally substituted with one or more R₅.

26. The method of any one of claims 1-20, wherein R₁ is H, OH, methyl,

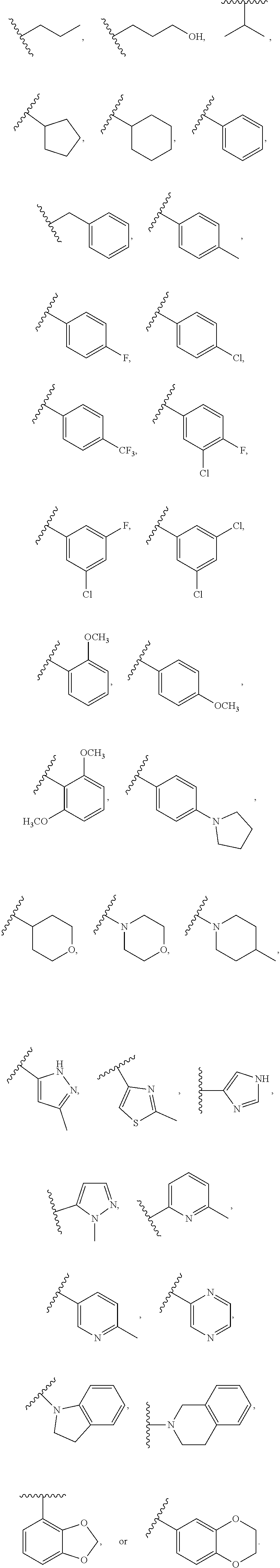

27. The method of any one of claims 1-20, wherein R₁ is H, OH, methyl,

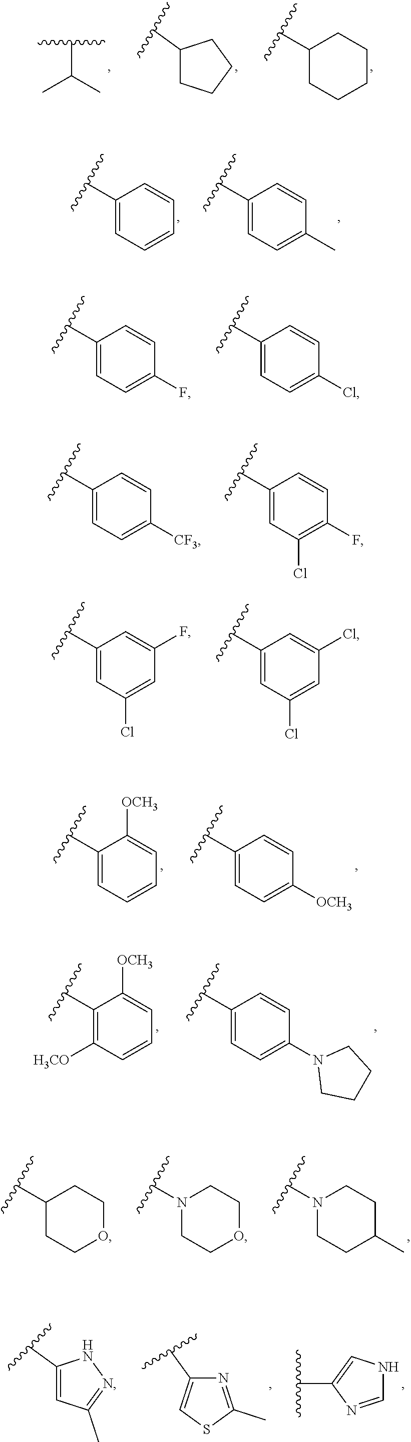

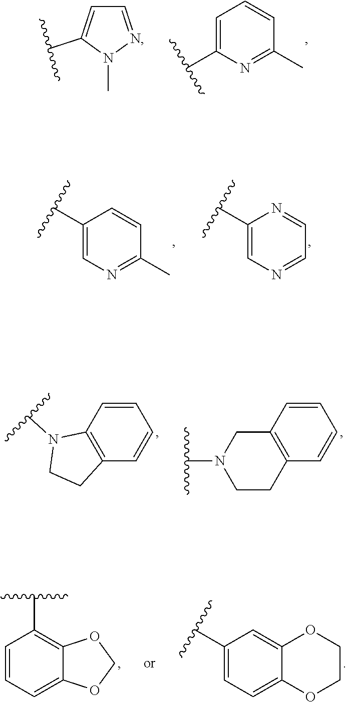

28. The method of any one of claims 1-20, wherein R₁ is

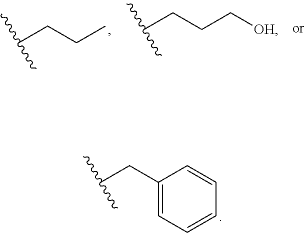

29. The method of any one of claims 1-20, wherein R₁ is H, OH, methyl,

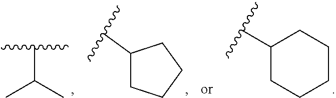

30. The method of any one of claims 1-20, wherein R₁ is

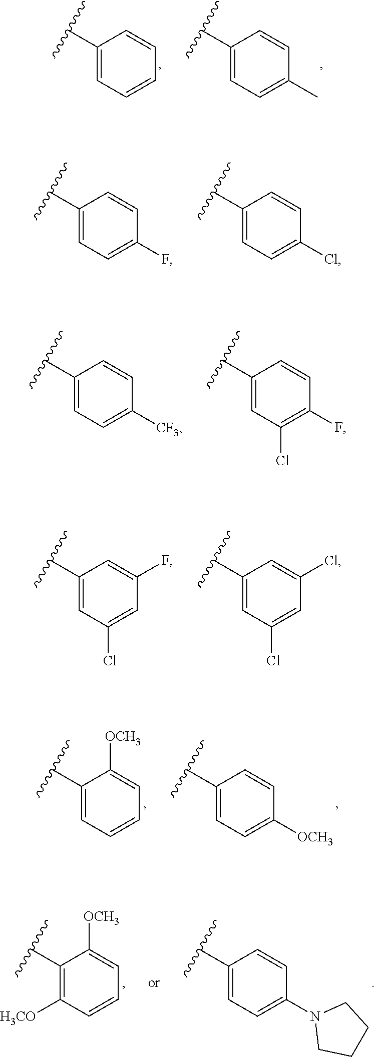

31. The method of any one of claims 1-20, wherein R₁ is

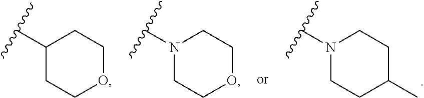

32. The method of any one of claims 1-20, wherein R₁ is

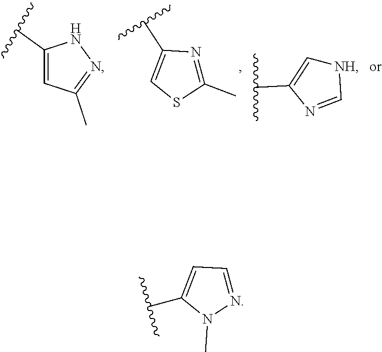

33. The method of any one of claims 1-20, wherein R₁ is

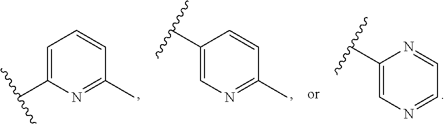

34. The method of any one of claims 1-20, wherein R₁ is

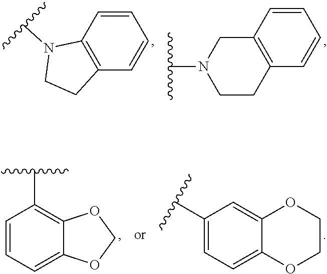

35. The method of any one of claims 1-34, wherein R₂ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, —C(O) C₁-C₆alkyl, —C(O)OC₁-C₆alkyl, —C(O)NH₂, —C(O)NHC₁-C₆alkyl, —C(O)N(C₁-C₆alkyl)₂, C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the alkyl, alkenyl, alkynyl, haloalkyl, alkoxyl, haloalkoxyl, cycloalkyl, aryl, heteroaryl, or heterocycloalkyl of any of the foregoing is optionally substituted with one or more R₆.

36. The method of any one of claims 1-34, wherein R₂ is C₆-C₁₀ aryl, 5- to 12-membered heteroaryl comprising 1-6 heteroatoms selected from N, O, and S, or 5- to 14-membered saturated or unsaturated heterocycloalkyl comprising 1-5 heteroatoms selected from N, O, and S; wherein the aryl, heteroaryl, or heterocycloalkyl of any of the foregoing is optionally substituted with one or more R₆.

37. The method of any one of claims 1-34, wherein R₂ is C₆-C₁₀ aryl, wherein the aryl is substituted with one or more R₆.

38. The method of any one of claims 1-34, wherein R₂ is 5- to 12-membered heteroaryl comprising 1-6 heteroatoms selected from N, O, and S; wherein the heteroaryl is substituted with one or more R₆.

39. The method of any one of claims 1-34, wherein R₂ is 5- to 14-membered saturated or unsaturated heterocycloalkyl comprising 1-5 heteroatoms selected from N, O, and S; wherein the heterocycloalkyl is substituted with one or more R₆.

40. The method of any one of claims 1-34, wherein R₂ is

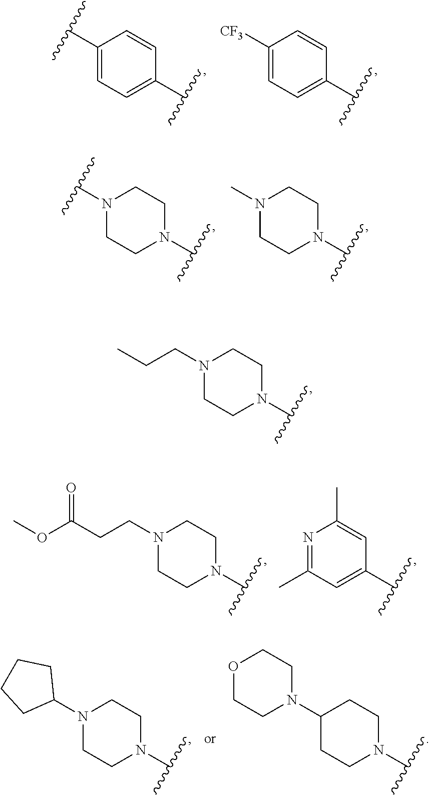

41. The method of any one of claims 1-34, wherein R₂ is

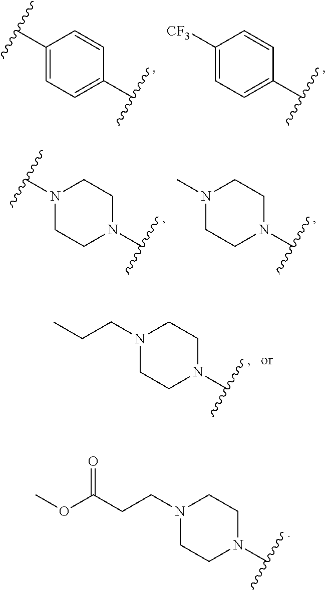

42. The method of any one of claims 1-34, wherein R₂ is

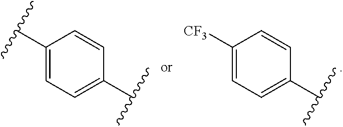 [SMILES: *c1ccc(*)cc1 ; *c1ccc(C(F)(F)F)cc1]

43. The method of any one of claims 1-34, wherein R₂ is

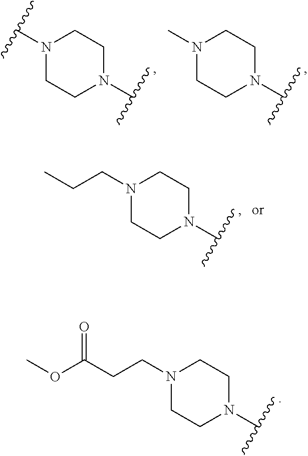

44. The method of any one of claims 1-34, wherein R₂ is

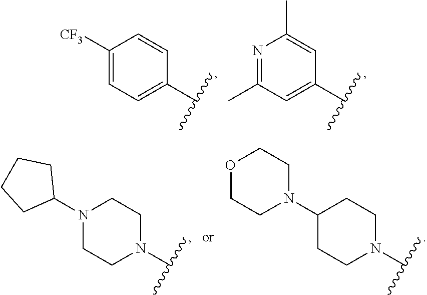

45. The method of any one of claims 1-44, wherein R₃ is halogen.

46. The method of any one of claims 1-44, wherein R₃ is H.

47. The method of any one of claims 1-46, wherein R₄ is halogen.

48. The method of any one of claims 1-46, wherein R₄ is H.

49. The method of any one of claims 1-48, wherein m is 1, 2, or 3.

50. The method of any one of claims 1-48, wherein m is 1.

51. The method of any one of claims 1-48, wherein n is 1, 2, or 3.

52. The method of any one of claims 1-48, wherein n is 1.

53. The method of any one of claims 1-48, wherein n is 2.

54. The method of any one of claims 1-48, wherein m is 1 and n is 1.

55. The method of any one of claims 1-48, wherein m is 1 and n is 2.

56. The method of any one of claims 1-55, wherein q is 1, 2, or 3.

57. The method of any one of claims 1-55, wherein q is 1.

58. The method of any one of claims 1-57, wherein R₅ is OH, halogen, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₆ cycloalkyl, or C₆-C₁₀ aryl, wherein the alkyl, alkenyl, alkynyl, cycloalkyl, or aryl of any of the foregoing is optionally substituted with one or more —C(O) C₁-C₆ alkyl, —C(O)OC₁-C₆alkyl, —C(O)NH₂, —C(O)NHC₁-C₆ alkyl, or —C(O)N(C₁-C₆alkyl)₂.

59. The method of any one of claims 1-57, wherein R₅ is OH or halogen.

60. The method of any one of claims 1-57, wherein R₅ is C₁-C₆ alkyl, C₂-C₆ alkenyl, or C₂-C₆ alkynyl.

61. The method of any one of claims 1-57, wherein R₅ is C₁-C₆ haloalkyl, C₁-C₆ alkoxyl, C₁-C₆ haloalkoxyl, or C₃-C₆ cycloalkyl.

62. The method of any one of claims 1-57, wherein R₅ is —C(O)OC₁-C₆ alkyl, —C(O)NH₂, —C(O)NHC₁-C₆ alkyl, or —C(O)N(C₁-C₆alkyl)₂.

63. The method of any one of claims 1-62, wherein R₆ is OH or halogen.

64. The method of any one of claims 1-62, wherein R₆ is C₁-C₆ alkyl, C₂-C₆ alkenyl, or C₂-C₆ alkynyl.

65. The method of any one of claims 1-62, wherein R₆ is C₁-C₆ haloalkyl, C₁-C₆ alkoxyl, C₁-C₆ haloalkoxyl, or C₃-C₆ cycloalkyl.

66. The method of any one of claims 1-62, wherein R₆ is —C(O)OC₁-C₆alkyl, —C(O)NH₂, —C(O)NHC₁-C₆alkyl, or —C(O)N(C₁-C₆alkyl)₂.

67. The method of any one of claims 1-62, wherein R₆ is a 5- to 14-membered saturated or unsaturated heterocycloalkyl comprising 1-5 heteroatoms selected from N, O, and S; wherein the heterocycloalkyl is optionally substituted with one or more —C(O) C₁-C₆alkyl, —C(O)OC₁-C₆alkyl, —C(O)NH₂, —C(O)NHC₁-C₆alkyl, or —C(O)N(C₁-C₆alkyl)₂.

68. The method of any one of claims 1-62, wherein R₆ is

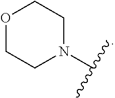 [SMILES: *N1CCOCC1]

69. The method of any one of claims 1-68, wherein R_{z} is C₁-C₆ alkyl.

70. The method of any one of claims 1-68, wherein R₇ is methyl.

71. The method of any one of claims 1-68, wherein R₈ is C₁-C₆ alkyl.

72. The method of any one of claims 1-68, wherein R₈ is methyl.

73. The method of any one of claims 1-68, wherein R₇ and R₈ are each methyl.

74. The method of any one of claims 1-68, wherein R₇ and R₈ together with the carbon atom they are attached, form a C₃-C₆ cycloalkyl.

75. The method of claim 1, wherein the compound is a compound of Formula (II): 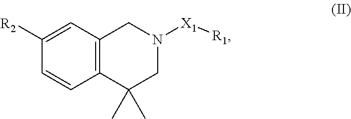
or a pharmaceutically acceptable salt thereof, wherein X₁, R₁, and R₂ are as described in claim 1.

76. The method of claim 75, wherein the compound is a compound of Formula (IIa): 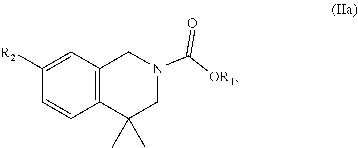
or a pharmaceutically acceptable salt thereof.

77. The method of claim 75, wherein the compound is a compound of Formula (IIb): 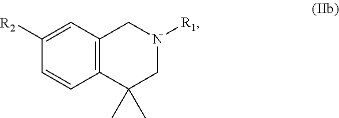
or a pharmaceutically acceptable salt thereof.

78. The method of claim 75, wherein the compound is a compound of Formula (IIc): 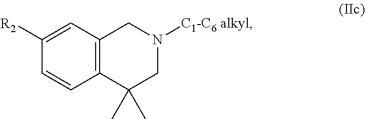
or a pharmaceutically acceptable salt thereof.

79. The method of claim 75, wherein the compound is a compound of Formula (IId): 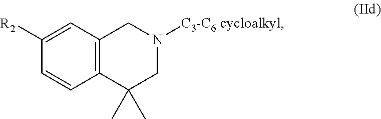
or a pharmaceutically acceptable salt thereof.

80. The method of claim 75, wherein the compound is a compound of Formula (IIe): 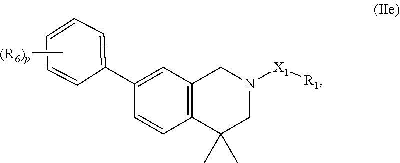
or a pharmaceutically acceptable salt thereof, wherein R₆ and p are as described in claim 1.

81. The method of claim 80, wherein the compound is a compound of Formula (IIf): 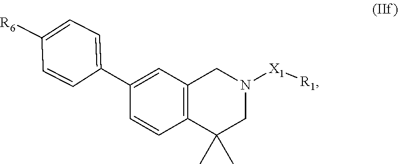
or a pharmaceutically acceptable salt thereof.

82. The method of claim 75, wherein the compound is a compound of Formula (IIg): 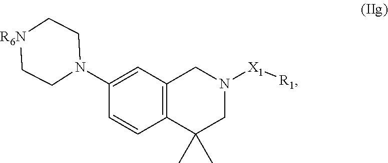
or a pharmaceutically acceptable salt thereof, wherein XR₅ is as described in claim 1.

83. The method of claim 82, wherein the compound is a compound of Formula (IIh): 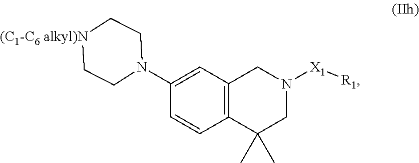
or a pharmaceutically acceptable salt thereof, wherein X₁, R₁, R₃, and R₄ are as described in claim 1.

84. The method of claim 1, wherein the compound is a compound of Formula (IIj): 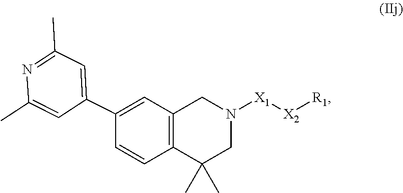
or a pharmaceutically acceptable salt thereof, wherein X₁, X₂, and R₁ are as described herein for Formula (I).

85. The method of claim 1, wherein the compound is a compound of Formula (IIk): 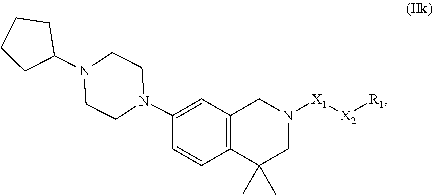
or a pharmaceutically acceptable salt thereof, wherein X₁, X₂, and R₁ are as described herein for Formula (I).

86. The method of claim 1, wherein the compound is a compound of Formula (III): 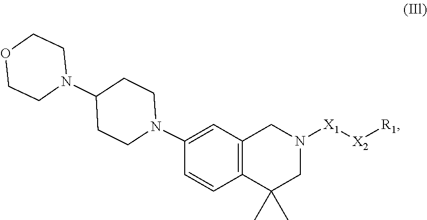
or a pharmaceutically acceptable salt thereof, wherein X₁, X₂, and R₁ are as described herein for Formula (I).

87. The method of claim 1, wherein the compound is a compound of Formula (III): 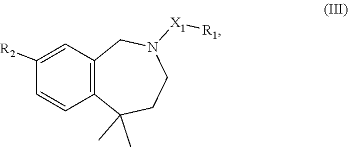
or a pharmaceutically acceptable salt thereof, wherein X₁, R₁, and R₂ are as described in claim 1.

88. The method of claim 87, wherein the compound is a compound of Formula (IIIa): 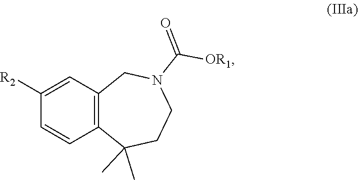
or a pharmaceutically acceptable salt thereof.

89. The method of claim 1, wherein the compound is a compound of Formula (IIIb): 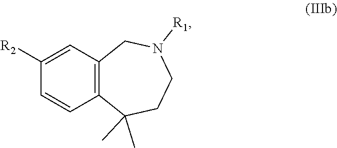
or a pharmaceutically acceptable salt thereof.

90. The method of claim 89, wherein the compound is a compound of Formula (IIIc): 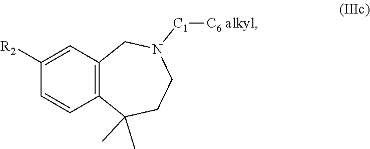
or a pharmaceutically acceptable salt thereof.

91. The method of claim 89, wherein the compound is a compound of Formula (IIId): 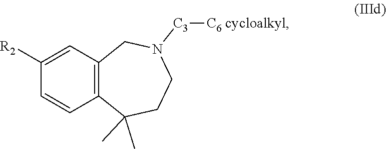
or a pharmaceutically acceptable salt thereof.

92. The method of claim 87, wherein the compound is a compound of Formula (IIIe): 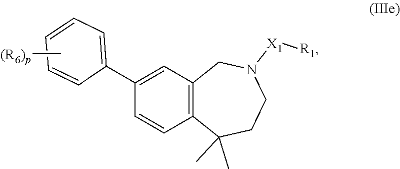
or a pharmaceutically acceptable salt thereof, wherein R₆ and p are as described in claim 1.

93. The method of claim 92, wherein the compound is a compound of Formula (IIIf): 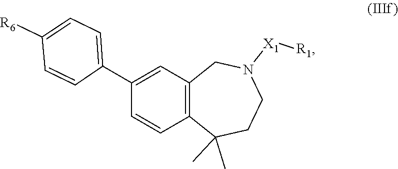
or a pharmaceutically acceptable salt thereof.

94. The method of claim 87, wherein the compound is a compound of Formula (IIIg): 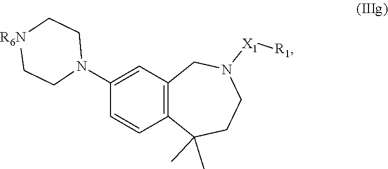
or a pharmaceutically acceptable salt thereof, wherein R₅ is as described in claim 1.

95. The method of claim 94, wherein the compound is a compound of Formula (IIIh): 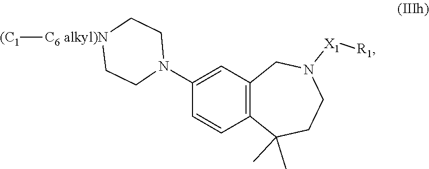
or a pharmaceutically acceptable salt thereof.

96. The method of claim 1, wherein the compound is a compound of Formula (IIIi): 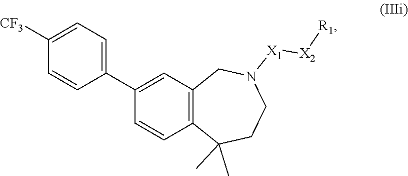
or a pharmaceutically acceptable salt thereof, wherein X₁, X₂, and R₁ are as described herein for Formula (I).

97. The method of claim 1, wherein the compound is a compound of Formula (IIIj): 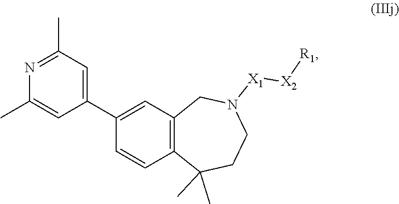
or a pharmaceutically acceptable salt thereof, wherein X₁, X₂, and R₁ are as described herein for Formula (I).

98. The method of claim 1, wherein the compound is a compound of Formula (IIIk): 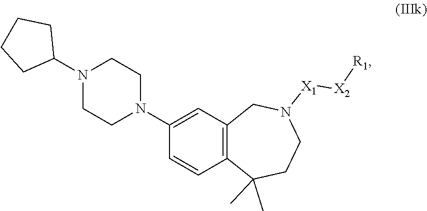
or a pharmaceutically acceptable salt thereof, wherein X₁, X₂, and R₁ are as described herein for Formula (I).

99. The method of claim 1, wherein the compound is a compound of Formula (IIII): 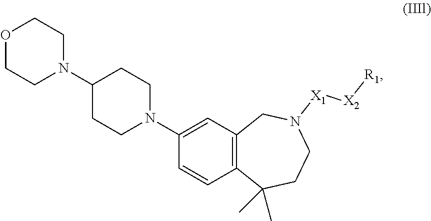
or a pharmaceutically acceptable salt thereof, wherein X₁, X₂, and R₁ are as described herein for Formula (I).

100. The method of claim 1, wherein the compound is any one of compounds 1-137 of Table 1, or a pharmaceutically acceptable salt thereof.

101. The method any one of claims 1-100, wherein the lysosomal storage disorder is cholesteryl ester storage disease, Wolman disease, Hunter syndrome, Hurler's disease, Scheie syndrome, Hurler-Scheie syndrome, mucopolysaccharidosis (MPS) type III, mucopolysaccharidosis (MPS) type IV (Morquio syndrome), Maroteaux-Lamy syndrome, Sly syndrome, Fabry disease, Gaucher disease, Krabbe disease, metachromatic leukodystrophy, Niemann-Pick disease, Sandhoff disease, Tay-Sachs disease, alpha mannosidosis, beta mannosidosis, Schindler disease, aspartylglucosaminuria, fucosidosis, neuronal ceroid lipofuscinosis, galactosialidosis, infantile sialic acid storage disease, Salla disease, sialuria, mucolipidosis, cystinosis, Danon disease, multiple sulfatase deficiency, proteins GM2-AP deficiency, Farber disease, lysosomal acid lipase deficiency, hyaluronidase deficiency, pycnodysostosis, and/or Pompe disease.

102. The method any one of claims 1-100, wherein the lysosomal storage disorder is Niemann-Pick disease.

103. The method any one of claims 1-102, wherein the lysosomal storage disorder is Niemann-Pick disease type C.

104. The method of any one of claims 1-102, wherein the lysosomal storage disorder is Niemann-Pick disease type A or B.

105. The method of any one of claims 1-104, wherein the subject has dyslipidemia.

106. The method of claim 105, wherein the dyslipidemia is high cholesterol.

107. The method of claim 105, wherein the dyslipidemia is low cholesterol.

108. The method of any one of claims 1-107, wherein the administration of the compound results in a modulation of cholesterol accumulation in the subject; and/or a modulation of the lysosomal size of the subject.

109. The method of claim 108, wherein the modulation is an increase in both cholesterol accumulation and lysosomal size.

110. The method of claim 108, wherein the modulation is a decrease in both cholesterol accumulation and lysosomal size.

111. The method of claim 108, wherein the modulation is an increase in cholesterol accumulation and a decrease in lysosomal size.

112. The method of claim 108, wherein the modulation is a decrease of cholesterol accumulation and an increase in lysosomal size.

113. The method of claim 108, wherein the cholesterol accumulation in the subject is determined by filipin staining.

114. The method of any one of claims 108-113, wherein the cholesterol accumulation is modulated by at least 10%, at least 20%, at least 30%, at least 40%, at least 50%, at least 60%, or at least 70%.

115. The method of any one of claims 108-114, wherein the modulation of cholesterol accumulation of the subject occurs within 24 hours, within 48 hours, or within 72 hours of the administration of the compound.

116. The method of any one of claims 108-115, wherein the lysosomal size is modulated by at least 10%, at least 20%, at least 30%, at least 40%, at least 50%, at least 60%, or at least 70%.

117. The method of any one of claims 108-116, wherein the modulation of lysosomal size of the subject occurs within 24 hours, within 48 hours, or within 72 hours of the administration of the compound.

118. The method of any one of claims 1-117, wherein the administration of the compound to the subject does not result in a change in NPC1 expression in the subject.

119. The method of any one of any one of claims 1-118, wherein the subject is a pediatric subject.

120. The method of any one of any one of claims 1-118, wherein the subject is an adult.

121. The method of any one of claims 1-120, wherein the subject is also administered one or more additional LSD treatment agents.

122. The method of claim 121, wherein the one or more additional LSD treatment agents is selected from recombinant enzymes, enzymes plus chaperones, recombinant enzymes plus chaperones, fusion proteins (HIRMAb), miglustat, and insulin growth factor II fusion protein.

123. The method of any one of claims 1-122, wherein the treatment or prevention of an LSD or a symptom of an LSD further comprises gene therapy.

124. The method of any one of claims 1 to 123, wherein the therapeutically effective amount of the compound is administered to the subject once per day.

125. The method of any one of claims 1 to 123, wherein the therapeutically effective amount of the compound is administered to the subject twice per day.

126. The method of any one of claims 1 to 125, wherein the therapeutically effective amount of the compound is between about 1 mg and about 1000 mg.

127. The method of any one of claims 1 to 126, wherein the therapeutically effective amount of the compound is about 10 mg, about 50 mg, about 100 mg, about 200 mg, about 300 mg, about 400 mg, about 500 mg, about 600 mg, about 700 mg, about 800 mg, or about 900 mg.
